SEQUENCE LISTING (78 sequences)

SEQ ID NO: 1 - DNA, 6689 bp, Homo sapiens
  gene (1)..(6689): the AIPL1 gene produces aryl-hydrocarbon receptor interacting protein-like 1
ggcctcccaaagtgctggattacaggcgtgagtcaccgcgcctggtcccctgtcttctttaagaaagctcagcggacctttttccttcttggggtggaacaaaaagccaaatctagcacaaccctgggcaggggcccagaatcactggaagcaaaggtggatgggataggaggcgaggctgcctgtggaccacaggcccggcccgagtggctctgatgagaagccggggcgcctaggtcaccgcccccaccgtctgcccttccccccactcctcctggctgggtaaatcccagagtctcagccgcctaagtgtcttccccggaggtgagattatctccgcctgtgctggacacctccctttctcctgcagccatggatgccgctctgctcctgaacgtggaaggggtcaagaaaaccattctgcacgggggcacgggcgagctcccaaacttcatcaccggatcccgagtgagtggggcccctccggagcagacagggtcccccacagcagctttcaacattccaggtgtgccccaaggcactgtaaacagctttcagctgtgccaaaaaaacagccaggcagccccagcgctgggcctccggggagctcccagcgtttacccattcagggggcatttttggtactttgcagattcaactttagcatgggctgaggggaagggcttttgggaattttctggggccctaaatgttgagtgagaagaaagggagtccgaggagtcttggtatttgtccccaaatgtctgttaggcttccctggactgaagggtgcgtctgtggctacagaattcgggctttggccaggcgaggcggctcccgcctgtaatcccagcactttgggaggccaagatgggcagatcatgaggtcaagagttcgagaccagcctgaccaacatgtgaaaccccatctctactgaaaatacaaaaattagccagatgtgctgtggcgcctgtaatcccagttcagatactcaggagacttgaggcaggagaatcacttgagcccaggaggtggaggttgcagtgagccgagatcataccactgcactccaacctgggcaacagagtgagactctgtctcagaaaaaaaaaaaaaaaaaagaactcgggcttacttgaggaaggatttctggacgcacagggctgtggggagtggaatggggtctgtagggaggggtgggtccctcctccctggggggtgcaggcagggtggaggtgctccaggggtctgaggcatctgatggggtgaactgagtgagctgaccctggggacagccctgggtgtcggtggcaagggggtggcttctgccgggccttgaacagtgtgtctagagcagagtgcaccgtctcggtgactaggtgatctttcatttccgcaccatgaaatgtgatgaggagcggacagtcattgacgacagtcggcaggtgggccagcccatgcacatcatcatcggaaacatgttcaagctcgaggtctgggagatcctgcttacctccatgcgggtgcacgaggtggccgagttctggtgcgacaccatcgtaagtaggccctgcgcgcctgtctcctgggactagtcttttctgggctcacccacccgctttgcggggctgctgtgtttcgggaaagctgggactcaagcgaagctttgcaaagccagtcctgcaaacttattccccaccgtgtgcatgtgaagatggagggaacaagggctggaaggggtgacccatgctgtggctggctggtggggagcagggctatgaccagcaggagtgagctggcccacttcacagtcctcacatctgtgtgtgtgtgtgtgtgtgtgtgtgtgtgtgtgtgtgtgtgtgtgtgagagagagagagagagagagagagagnnnnnnnnnntagccttaggacttattgcagagaccaacacctaacaatgtaatcaggcagccagtgcaggacataaataagtaaggcagtgtgctttgggccacaaaagcacgctcagcttgctggaagccatgggtgccgagctgggggctgctgagtcagggccaaagggggcccctccctgcagtaagctggttctggggcctctccctcccttggtccagctcttaatcccaacaggctcaacagccatctgcttgtctcttccataaagaggcagaaggcatttcgggctaatcccggccggtggggcgggcagggtgacctctgtctctgtgctggtgacctggaggcagagctgaactgctgcatagagtttcagccccttcacttcacatgttgcatgtggggccagtgctgggtcatctcagaagccggtccaaggagatgggttctcagggagcctagttggggaaactgaggcccagcatacatacagcaggcctcgctgaggccgcacggcggatcttcccagccctccttcatcccaagggtggcaaactcagctcccatgctggctgaagctgtgatgagccagatctatatctgcaccatctcatttaatccctacagcagccctaatatcgaacaggagcaacccagggaactgagtttcagagaagtgcagagacctgggctcaccgctaacctgcagcactgccaggacaccaaagcgactctcttggaccctggagtcctgctccttctactgccccacactgcccttcctgcgagtcataggctttgcagaggtcagggtttccctggggcagagatgtgttacagtggaccacaagggccagaagaggcagccggaggctaacagcatatggcctctggagccaggtttgaatcctggctgcgtcatttcctagctgtgtgaccttaagcaagttgcttgcgtctctgggctgtagtttccccatccgtaaaatgggataatagtgcctgccttgaattgtcataaggattgaaggggctcataacagtgtgaagtgctttgcctggcacacagttaaccacagttagtatgagtggcatagtgagggagcaggattcctcccaggaggggctctgagtggaggccttttatggcccacctagctctgggcaggtagcctggatgccatccatccgtttatccccacagcacacgggggtctaccccatcctrtcccggagcctgaggcagatggcccagggcaaggaccccacagagtggcacgtgcacacgtgcgggctggccaacatgttcgcctaccacacgctgggctacgaggacctggacgagctgcagaaggagcctcagcctctggtctttgtgatcgagctgctgcaggtggggctggggttggcagggctggagggctgtgccagcactggagagggacagcgggcatcatgggcacccccaccccactggccactggacagtgccctgtttctgtttagataatacgagagggttcataagccatgggagaatacgaatttgaaaaaaaagtcctctgatttttccacaagaaaagtcctttggtgctgggcatggtggcccacgcctgtaatcctagcactttgggaggccgagggggttggatcacctgaggtcaggagttcgaagaccagcctggccaacatggtaaaaccccgtctctattaaaaacacaaaaattaaccgggtgtggtggtgcatgcctgtaatcaatcccagctacttgggaatttgaggcatgagaattgcttgaacctggaagtggaggttgcagtgagcagagatcatgtcagtgcattttaacctgggtgacagagtgagactccatgtccaaaaaaaagaaaaaaaaaaaaagtccacttggaaccagtttttaaaaatgtgattcattttcattgtggaggcattttatccacttccactttcattttcaggagttggagattataaccgcctccttggttcctgtggtttgtgggttcagacttggttctctngtggcgggagaggctgcatggaactccccacatcctcccaaccaggagccccagagtgattggcagcgcgtgtttgtggattggtgagagagggttagggccagggtcaaggtcaggtcaggactcagcttatggccaagactgaggctcagcctgagagctatgtgggtgaataaaataaaataagaactgtgtcaaccaagggccccttacaggcttgctgtcacagttgtgtggtctgtgcactgcacaaggtgcaccggcatctcctccaaggtgctcattatagacattgtatattggtatttccataatgagaagtttccagcagatggcaatagtgtattgttctaacaaaacgagtattcgtgacaattttctgaatattagaagtgaagtgtcttgatgaacgggcaccttttcctagtttgcacaaagacattgatttagggcagggttttcggcgttgttgcttctttcccttgtctgtatgcacttgaccagcaagcatgacttcagggagatgtgccacagggtcctgtttttcgggtctctgatggggtgcaggcccctggggtccctgcctcactgacctgcagctctggggccaggttgatgccccgagtgattaccagagggagacctggaacctgagcaatcatgagaagatgaaggcggtgcccgtcctccacggagagggaaatcggctcttcaagctgggccgctacgaggaggcctcttccaagtaccaggaggccatcatctgcctaaggaacctgcagaccaaggtcagaggccgctggccaggggtgggaagtggcgctgactctggggggcctgcccagtgccggccagggtggggcgggggttgggcagctgcctgaggtcatggctgaccttctccctgggcaggagaagccatgggaggtgcagtggctgaagctggagaagatgatcaatactctgatcctcaactactgccagtgcctgctgaagaaggaggagtactatgaggtgctggagcacaccagtgatattctccggcaccacccaggtgcgcggggctgcaggggcggacagtgagggggcgcccagcccagggccacggagacacctgccatagccttcctggacttttctttccaccccaccagggcaccaaaccttgtctccacccagccgggtttccccgagtgtgtaactgaattgtgggtgatggatgggcagtgcttggcgcggggcggcctttattttaatgtgtgtttgaacacttacccaggaagctcgccaagcttgtgatttcagcggaacggtaaacaggcgtttaaaaagaggggcaatcaatatagggaaaaatattatgatgtcggtactagtactggtgttgcgaggatatggcaccgcagtactagattgacttaatgctcgaatcgtgctcacagtaaaaacatccagcccctggctcatgcatcaggcacacgtcgtctgcgtttattatctcatttaatcctcataatcctcataatcaccatatgagggaggtgcagggaaaggggcctgaaggttatctaatttaggtagcgtctataagaaaaataaaacaaagttatgaatataaaattactcacagggccttaaaaaggagaggaggaggtactgctattatgatcatcatctccatcttacagttgaggaaaccgagggatgggggatacagagaggttaaggatcatggcggggctgagggtcttggaggctggtgagtcccagctgggctggggctgcctctgaggctgggaagggagctgtagctggatgctccctgctccccacaggcatcgtgaaggcctactacgtgcgtgcccgggctcacgcagaggtgtggaatgaggccgaggccaaggcggacctccagaaagtgctggagctggagccgtccatgcagaaggcggtgcgcagggagcttgaggctgctggagaaccgcatggcggagaacaggaggaggagcggctgcgctgccggaacatgctgagccagggtgccacgcagcctcccgcagagccacccacagagccacccgcacagtcatccacagagccacctgcagagccacccacagcaccatctgcagagctgtccgcagggccccctgcagagccagccacagagccacccccgtccccagggcactcgctgcagcactgagccccctgaggcccacagccacccaggcagggagcaagtggcctggtcacttctggttcgattgaccaggatcgtggtgtcactttttaaaatttaaaattaatttttgaaatcaaagtcagacacacccatggtaaaaaaaaaaaaaaaaacaatcccaagggtacagaagagcttatgaataaaagtagttttctcctctacccctctcattccttccgtgccatggttttaattgaccctgtttttaattcttctggtagttttctctatttccaagtaatctgtttaaatcagtttctagattttaccccatgtcaatgacaaatgaggatttgatgctctgatcctttctcatgcctgatacccctccctgtctccccattttggatagttacatttgggggtcatctcggtgatttttgtaactttacgcaggacacttagagctctctagaatcccactgactttagtggggtcttgatgtagggtgggcaagccccgacactggagcttagcctgagaggggttcttgc

SEQ ID NO: 2 - DNA, 1119 bp, Papio anubis
  gene (1)..(1119): the AIPL1 gene produces aryl-hydrocarbon receptor interacting protein-like 1
atggatgccgctctgctcctgaacgtggaaggggtcaagaaaaccattctgcacggaggcacgggcgagctcccaaacttcatcaccggatcccgagtgatctttcatttccgcaccatgaaatgtgatgaggagcgcacggtcatcgacgacagccggcaggtggaccagcccatgcacatcatcatcgggaacatgttcaagctcgaggtctgggagatcctgctcacctccatgagggtgcacgaggtggccgagttctggtgcgacaccatccacacgggggtctaccccatcctgtcccggagcctgcggcagatggcccagggcaaggaccccacggagtggcacgtgcacacatgcgggctggccaacatgttcgcctaccacacactgggctacgaggacctggacgagctgcagaaggagcctcagcctctgatctttgtgatcgagctgctgcaggttgacgccccgagtgattaccagagggagacctggaacctgagcaatcatgagaagatgaaggtggtgcccgtcctccacggagagggaaatcggctcttcaagctgggccgctacgaggaggcctcttccaagtaccaggaggccatcatctgcctaaggaacctgcagaccaaggagaagccatgggaggtgcagtggctgaagctggagaagatgatcaacaccctgaccctcaactactgccagtgcctgctgaagaaggaggagtattacgaggtgctggagcacaccagtgacattctccggcaccacccaggcatcgtgaaggcctactatgtgcgtgcccgggctcacgcagaggtgtggaatgaggccgaggccaaggcggacctccagaaagtgctggagctggagccatccatgcagaaggcggtgcgcagggagctgaggctgctggagaaccgcatggcagagaagcaggaggaggagcggctgcgctgccggaacatgctgagccagggagccacgcagcctcccacagagccaccggcagagccccacacagcaccacctgcggagctgtccacagggccacctgcagagccacccgcagagctccccctgtccccagggcactcactgcagcactga

SEQ ID NO: 3 - DNA, 1155 bp, Pan troglodytes
  gene (1)..(1155): the AIPL1 gene produces aryl-hydrocarbon receptor interacting protein-like 1
atggatgccgctctgctcctgaacgtggaaggggtcaagaaaaccattctgcacgggggcacgggcgagctcccaaacttcatcaccggatcccgagtgatctttcatttccgcaccatgaaatgtgatgaggagcggacagtcattgacgacagccggcaggtgggccagcccatgcacatcatcatcggaaacatgttcaagctcgaggtctgggagatcctgcttacctccatgcgggtgcacgaggtggccgagttctggtgcgacaccatccacacaggggtctaccccatcctgtcccggagcctgaggcagatggcccagggcaaggaccccacagagtggcacgtgcacacatgcgggctggccaacatgttcgcctaccacacgctgggctacgaggacctggacgagctgcagaaggagcctcagcctctggtctttgtgatcgagctgctgcaggttgatgccccgagtgattaccagagggagacctggaacctgagcaatcatgagaagatgaaggcggtgcccgtcctccacggrgagggaaatcggctcttcaagctgggacgctacgaggaggcctcttccaagtaccaggaggccatcatctgcctaaggaacctgcagaccaaggagaagccgtgggaggtgcagtggctgaagctggagaagatgatcaatactctgatcctcaactactgccagtgcctgctgaagaaggaggagtactatgaggtgctggagcacaccagcgacattctccggcaccacccaggcatcgtgaaggcctactacgtgcgtgcccgggctcacgcagaggtgtggaatgaggccgaggccaaggcagacctccggaaagtgctggagctggagccgtccatgcagaaggcggtgcgcagggagctgaggctgctggagaaccgcatggcggagaagcaggaggaggagcggctgcgctgccggaacatgctgagccagggtgccacgcagcctccggcagagccacccacagagccacccgcacagtcatccacagagccacctgcagagccacccccagcaccatctgcagagctgtccgcagggccacctgcagagacagccacagagccacccccgtccccagggcactcgctgcagcactga

SEQ ID NO: 4 - DNA, 1060 bp, Bos taurus
  gene (1)..(1060): the AIPL1 gene produces aryl-hydrocarbon receptor interacting protein-like 1
atggatgccactctgctcctgaatgtggaagggatcaagaaaaccattctgcatgggggcacaggggacctccccaacttcattactggagcccgagtgacctttcatttccgaaccatgaaatgtgatgaggagcggacggtgatagacgacagcaagcaggtgggccatcccatgcacatcatcattgggaacatgttcaagctggaggtctgggagatcttgctgacgtccatgcgggtcagcgaggtggccgagttttggtgcgacaccatccacacaggggtctaccccatcctgtcccggagcctgcggcagatggcggagggtaaggaccccacagagtggcacgtgcacacgtgtggcttggccaacatgttcgcttaccacacgctgggctacgaggacctggacgagctgcagaaggagcctcagccactgatcttcataatcgagttgctgcaggtcgaggccccgagccagtaccagagggagacctggaacctgaataaccaggagaagatgcaggcggtgcccatcctccatggagaaggaaaccggctcttcaagctgggccgctacgaggaggcctccaacaagtaccaggaagccatcgtctgcctgaggaacctgcagaccaaggagaaaccctgggaggtgcagtggctgaagctggagaagatgatcaacaccctgatcctgaactactgtcagtgtctgctgaagaaggaggagtactacgaggtgctggaacacactagtgacatcctccggcatcacccaggcatcgtgaaggcctactatgtgagggcccgggctcacgccgaggtgtggaatgaggcggaagccaaggcggatctggagaaagtgctggagctggagccgtccatgcggaaggcggtgcagagggagctgaggctgctggagaaccggctggaggagaaacgcgaggaggagcgactgcgctgccggaacatgctgggctagtgcgcaggcgccaagcctcctgcctccgccccccgcycctccaccccccccaaaaaaaaaaaaaaaaattttt

SEQ ID NO: 5 - DNA, 925 bp, Canis familiaris
  gene (1)..(925): the AIPL1 gene produces aryl-hydrocarbon receptor interacting protein-like 1
tgtacgggggcaccggcgagctcccaaacttcctcacggggtcccgggtcatctttcacttccgcacaacgaaatgcgacgaggcgcggacagtgatcgacgacagcaagcgtgtgggccatcccatgcacatcatcatcgggaacatgttcaagctggaggtctgggaggtgctgctgacatccatgcgcgtgggcgaggtggccgagttctggtgcgactctattcacacaggagtctaccccatcctgtcccggagcctgcggcaggtggcggagggcaaggaccccactgagtggcatgtacacacgtgcggcttggccaacatgtttgcctatcacacgctgggctacgaggacctggacgagctacagaaggagccgcagcccctcatcttcatgatagagctgctgcaggtggaggccccaagtgagtaccagagggagacgtggagcctgaacaatgagagaagatgcagcggtacccatctcatggagaggggaaccggctcttcaagctgggccgctacaatgatgcctccaccaagtaccaggagccatcgtctgctgaggaacctgcagaccaaggagaagcctgggaggtgcagtggctaaagctggagaagctgatcaacaccttgattctcaactactgccagtgtctgctgaagaaggaggagtactacgaggtgctggagcacactagcgacatcctgcggcttcacccaggaatcgtgaaggcctactacgtgcgcgcccgggctcacgcggaggtgtggaacgaggccgaggccagggcggaccttcagaaagtgctggagctggagccatccatggggaaggctgtgcgcagggagctgcggcttctggaaaatcgcctggaggaaaagcgggaggaggagcggctgcgctgccggaacatgcta

SEQ ID NO: 6 - DNA, 1075 bp, Mus musculus
  gene (1)..(1075): the AIPL1 gene produces aryl-hydrocarbon receptor interacting protein-like 1
atggacgtctctctactcctcaatgtggagggtgtcaagaagaccattctgcatggggggacaggagagctccccaacttcatcactggctccagagtgacctttcatttccgaacaatgaagtgtgatgaagaacgcacggtgatcgatgacagcaagcaggtgggccagcccatgagcatcatcatcggcaacatgttcaagctggaggtgtgggagacgctgctgacctccatgcggctgggcgaggtggctgagttctggtgcgacaccattcacacaggggtctaccctatgttgtcccgcagtctgcggcaggtggctgagggcaaggaccccacaagctggcatgtgcacacgtgcgggttggccaacatgtttgcataccacacgctgggctacgaggacctggatgagctgcagaaagagccacagcctcttgtcttcctgtatgaactgttgcaggtggaggccccaaatgagtaccagagggagacgtggaacctgaataatgaagagaggatgcaggcggtacctcttcttcatggagaaggcaacaggctctacaagctgggacgctatgatcaggccgccaccaagtaccaggaggccattgtgtgcctgaggaaccttcagaccaaggagaagccctgggaggttgagtggctgaagctggagaagatgatcaacaccctgatcctcaactactgccagtgcctgctgaagaaggaggagtactacgaggtgttggagcacaccagcgacattctacgacaccacccagggatcgtgaaggcctactatatgcgcgcacgtgctcacgcagaggtgtggaacgctgaggaggccaaggcggacctggagaaagtgctggagttggagccatccatgcgcaaggcggtgctcagggaactgcggctgctggagagccgcctggcggacaaacaggaggaggagcggcagcgctgccggagcatgctgggctaggctgggctggattccactgagttagactgggttaggttgggtgggagctgcgggttgaaccctggggcgagggctggggctatggact

SEQ ID NO: 7 - DNA, 1179 bp, Macaca mulatta
  gene (1)..(1179): the AIPL1 gene produces aryl-hydrocarbon receptor interacting protein-like 1
atggatgccgctctgctcctgaacgtggaaggggtcaagaaaaccattctgcacggaggcacgggcgagctcccaaacttcatcaccggatcccgagtgatctttcatttccgcaccatgaaatgtgatgaggagcgcacggtcatcgacgacagccgtcaggtggaccagcccatgcacatcatcatcgggaacatgttcaagctcgaggtctgggagatcctgctcacctccatgagggtgcacgaggtggccgagttctggtgcgacaccatccacacgggggtctaccccatyctgtcccggagcctgcggcagatggcccagggcaaggaccccacggagtggcacgtgcacacatgcgggctggccaacatgttcgcctaccacacgctgggctacgaggacctggacgagctgcagaaggagcctcagcctctgatctttgtgatcgagctgctgcaggttgacgccccgagtgattaccagagggagacctggaacctgagcaatcatgagaagatgaaggtggtgcccgtcctccacggagagggaaatcggctcttcaagytgggccgctacgaggaggcctcttccaagtaccaggaggccatcatctgcctaaggaacctgcagaccaaggagaagccgtgggaggtgcagtggctgaagctggagaagatgatcaacaccctgaccctcaactactgccagtgcctgctgaagaaggaggagtattacgaggtgctggagcacaccagtgacattctccggcaccacccaggcatcgtgaaggcctactatgtgcgtgcccgggctcacgcggaggtgtggaacgaggccgaggccaaggcggacctccagaaagtgctggagctggagccatccatgcagaaggcggtgcgcagggagctgaggctgctggagaaccgcatggcggagaagcaggaggaggagaggctgcgctgccggaacatgctgagccagggagccacgcagcctcccgcagagccaccggcacagccccccacagcaccacctgcagagctgtccacagggccacctgcggacccaccggcggagccccccacagcaccacctgcggagctgtccacagggccacctgcagagccacccgcagagctccccctgtccccagggcactcactgcagcactga

SEQ ID NO: 8 - DNA, 1119 bp, Saimiri sciureus
  gene (1)..(1119): the AIPL1 gene produces aryl-hydrocarbon receptor interacting protein-like 1
atggatgccgctctgctcctgaacgtggaaggggtcaagaagaccattctgcacgggggcacgggcgagctcccaaatttcatcaccggatcccgagtgatctttcatttccgcaccatgaaatgtgatgaggagcggacggtgattgacgacagcagggaggtgggccagcccatgcacatcatcatcgggaacatgttcaagctggaggtctgggagatcctgctcacgtccatgcgggtgcgagaggtggccgagttctggtgcgacaccatccacacgggggtctaccccatcctgtcccggagcctgcggcagatggcccagggcaaggacccgacggagtggcatgtgcacacgtgcgggctggccaacatgttcgcctaccacacgctgggctacgaggacctggatgagctgcagaaggagcctcagcctctgatctttgtgatcgagctgctgcaggttgatgccccaagtgattaccagagggagacctggaacctgagcaatcacgagaagatgaaggtggtgcccgtcctccatggagaaggaaataggctcttcaagctgggccgctacgaggaggcctcttccaagtaccaggaggccatcatctgcctaaggaacctgcagaccaaggagaaaccctgggaggtgcagtggctgaagctggagaagatgatcaataccctgatcctcaactactgtcagtgtctgctgaagaaggaggagtactacgaggtcctggagcataccagtgacattctccggcaccacccaggcattgtgaaggcctactatgtgcgcgcccgggctcacgcggaggtgtggaacgaggccgaggccaaggcggacctccagaaagtgctggagctggagccgtccatgcagaaggcggtgcgcagggagctgaggctgctggagaaccgcatggcggagaagcaggaggaggagcggctgcgctgccgcaacatgctgagccagggggccacgtggtcccccgcggagccacccgcagagccacctgcagagtcatccacagagccacccgcagagccacctgcagagccacctgcagagctaaccttgaccccggggcacccactacagcactga

SEQ ID NO: 9 - DNA, 15 bp, Homo sapiens
  mutation (7)..(9): Amino Acid codon position 79  Met to Thr mutation
acctccacgcgggtg

SEQ ID NO: 10 - DNA, 15 bp, Homo sapiens
  Mutation (7)..(9): Amino Acid condon 88 mutation Trp to X
gagttctgatgcgac

SEQ ID NO: 11 - DNA, 15 bp, Homo sapiens
  Mutation (7)..(9): Amino Acid condon Mutation position 96 Val to Ile
acggggatctacccc

SEQ ID NO: 12 - DNA, 15 bp, Homo sapiens
  mutation (7)..(9): Amino Acid codon mutation position 124  Thr to Ile
gaccccatagagtgg

SEQ ID NO: 13 - DNA, 15 bp, Homo sapiens
  Mutation (7)..(9): Amino Acid codon mutation position 376  Pro to Ser
ccaccctcgtcccca

SEQ ID NO: 14 - DNA, 15 bp, Homo sapiens
  mutation (7)..(9): Amino Acid codon mutation position 163 Gln to
gattactagagggag

SEQ ID NO: 15 - DNA, 15 bp, Homo sapiens
  mutation (7)..(9): Amino Acid codon mutation position 197 Ala to ro
gaggagccctcttcc

SEQ ID NO: 16 - DNA, 15 bp, Homo sapiens
  mutation (7)..(9): Amino Acid codon mutation  TRP 278  X
gaggtgtgaaatgag

SEQ ID NO: 17 - DNA, 15 bp, Homo sapiens
  mutation (7)..(7): a to g mutation IVS2-2A to G
tccccacggcacacg

SEQ ID NO: 18 - DNA, 15 bp, Homo sapiens
  mutation (7)..(9): Amino Acid codon mutation Glu 262 Ser
cacccaagtgcgcgg

SEQ ID NO: 19 - DNA, 15 bp, Homo sapiens
  mutation (7)..(9): Amino Acid codon mutation Arg 302 Leu
gcggtgctcagggag

SEQ ID NO: 20 - DNA, 13 bp, Homo sapiens
  mutation (5)..(5): deletion of  “TGCAGAGCCACC” sequence
gccacccacagca

SEQ ID NO: 21 - DNA, 15 bp, Homo sapiens
  mutation (7)..(9): Amino Acid codon mutation Cys 239 Arg
tgccagcgcctgctg

SEQ ID NO: 22 - DNA, 13 bp, Homo sapiens
  mutation (5)..(5): two base deletion “AG”
tcccgcagccacc

SEQ ID NO: 23 - DNA, 15 bp, Homo sapiens
  mutation (7)..(9): Amino Acid codon mutation Cys 42 X
atgaaatgagatgag

SEQ ID NO: 24 - DNA, 12 bp, Homo sapiens
  mutation (7)..(7): nine base deletion “CTCCGGCAC”
gatattcaccca

SEQ ID NO: 25 - DNA, 21 bp, Homo sapiens
  mutation (7)..(7): eight base insertion +37GTGATCTT”
gactaggtgatcttgtgatct

SEQ ID NO: 26 - DNA, 12 bp, Homo sapiens
  mutation (4)..(4): g to a polymorphism IVS1-9G to A Benign
ctcagtgactag

SEQ ID NO: 27 - DNA, 12 bp, Homo sapiens
  mutation (4)..(4): g to c polymorphism  IVS2+66G to C Benign
tttgccgggctg

SEQ ID NO: 28 - DNA, 12 bp, Homo sapiens
  mutation (4)..(4): c to t polymorphism IVS2-88C to T Benigh
tcctctcaggag

SEQ ID NO: 29 - DNA, 12 bp, Homo sapiens
  mutation (4)..(4): g to a polymorphism IVS2-14G to A Benign
atccatttatcc

SEQ ID NO: 30 - DNA, 12 bp, Homo sapiens
  mutation (4)..(4): a to c mutation  IVS2-10A to C Benign
cgtttctcccca

SEQ ID NO: 31 - DNA, 12 bp, Homo sapiens
  mutation (4)..(4): t to c mutation IVS3-25T to C Benign
ctgccccactga

SEQ ID NO: 32 - DNA, 12 bp, Homo sapiens
  mutation (7)..(7): t to c mutation IVS3-21T to C Benign
cctcaccgacct

SEQ ID NO: 33 - DNA, 12 bp, Homo sapiens
  mutation (7)..(7): g to a mutation IVS5+3018G to A Benign
aggagcggacag

SEQ ID NO: 34 - DNA, 12 bp, Homo sapiens
  mutation (7)..(9): Amino Acid codon mutation Asp 90 His Benign
tggtgccacacc

SEQ ID NO: 35 - DNA, 12 bp, Homo sapiens
  mutation (4)..(6): Amino Acid mutation Phe 37 Phe Benign
catttccgcacc

SEQ ID NO: 36 - DNA, 12 bp, Homo sapiens
  mutation (4)..(6): Amino Acid mutation Ser 78 Ser Benign
acctctatgcgg

SEQ ID NO: 37 - DNA, 12 bp, Homo sapiens
  mutation (4)..(6): Amino Acid mutation Cys 89 Cys Benign
tggtgtgacacc

SEQ ID NO: 38 - DNA, 12 bp, Homo sapiens
  mutation (4)..(6): Amino Acid codon mutation  Leu 100 Leu Benign
atcctgtcccgg

SEQ ID NO: 39 - DNA, 12 bp, Homo sapiens
  mutation (4)..(6): Amino Acid codon mutation His 172 His
aatcacgagaag

SEQ ID NO: 40 - DNA, 12 bp, Homo sapiens
  mutation (4)..(6): Amino Acid codon mutation Pro 217 Pro Benign
aagccgtgggag

SEQ ID NO: 41 - DNA, 12 bp, Homo sapiens
  mutation (4)..(6): Amino Acid codon mutation Asp 255 Asp Benign
agtgacattctc

SEQ ID NO: 42 - DNA, 20 bp, Homo sapiens
  primer_bind (1)..(20): 5′ to 3′ primer sequence
aagaaaaccattctgcacgg

SEQ ID NO: 43 - DNA, 19 bp, Homo sapiens
  primer_bind (1)..(19): 5′ to 3′ primer sequence
tgcagctcgtccaggtcct

SEQ ID NO: 44 - DNA, 17 bp, Homo sapiens
  primer_bind (1)..(17): 5′ to 3′ primer sequence
gacacctccctttctcc

SEQ ID NO: 45 - DNA, 18 bp, Homo sapiens
  primer_bind (1)..(18): 5′ to 3′ primer sequence
gctggggctgcctggctg

SEQ ID NO: 46 - DNA, 20 bp, Homo sapiens
  primer_bind (1)..(20): 5′ to 3′ primer sequence
ccgagtgattaccagaggga

SEQ ID NO: 47 - DNA, 20 bp, Homo sapiens
  primer_bind (1)..(20): 5′ to 3′ primer sequence
tgagctccagcacctcatag

SEQ ID NO: 48 - DNA, 18 bp, Homo sapiens
  primer_bind (1)..(18): 5′ to 3′ primer sequence
acgcagaggtgtggaatg

SEQ ID NO: 49 - DNA, 19 bp, Homo sapiens
  primer_bind (1)..(19): 5′ to 3′ primer sequence
aaaaagtgacaccacgatc

SEQ ID NO: 50 - DNA, 34 bp, Homo sapiens
  misc_binding (1)..(34): exon/intron - donor splice site CGGATCCCGAgtgagtggggccctccggagca ga
cggatcccgagtgagtggggccctccggagcaga

SEQ ID NO: 51 - DNA, 35 bp, Homo sapiens
  misc_binding (1)..(35): exon/intron Acceptor splice site cagagtgcaccgtctcggtgactagGTGATC TTTC
cagagtgcaccgtctcggtgactaggtgatctttc

SEQ ID NO: 52 - DNA, 35 bp, Homo sapiens
  misc_binding (1)..(35): exon/intron Donor splice site CSACACCATCgtaagtaggccctgcgcgcctgtc t
csacaccatcgtaagtaggccctgcgcgcctgtct

SEQ ID NO: 53 - DNA, 35 bp, Homo sapiens
  misc_binding (1)..(35): exon/intron Acceptor splice site gccatccatccgtttatccccacagCACACG GGGG
gccatccatccgtttatccccacagcacacggggg

SEQ ID NO: 54 - DNA, 35 bp, Homo sapiens
  misc_binding (1)..(35): exon/intron Donor splice site GCTGCTGCAGgtggggctggggttggcagggct gg
gctgctgcaggtggggctggggttggcagggctgg

SEQ ID NO: 55 - DNA, 35 bp, Homo sapiens
  misc_binding (1)..(35): exon/intron Acceptor splice site cactgacctgcagctctggggccagGTTGA TGCCC
cactgacctgcagctctggggccaggttgatgccc

SEQ ID NO: 56 - DNA, 35 bp, Homo sapiens
  misc_binding (1)..(35): exon/intron Donor splice site GCAGACCAAGgtcagaggccgctggccacggggt g
gcagaccaaggtcagaggccgctggccacggggtg

SEQ ID NO: 57 - DNA, 35 bp, Homo sapiens
  misc_binding (1)..(35): exon/intron Acceptor splice site catggctgaccttctccctgggcagGAGAA GCCRT
catggctgaccttctccctgggcaggagaagccrt

SEQ ID NO: 58 - DNA, 35 bp, Homo sapiens
  misc_binding (1)..(35): exon/intron Donor splice site CACCACCCAGgtgcgcggggctgcaggggcgga ca
caccacccaggtgcgcggggctgcaggggcggaca

SEQ ID NO: 59 - DNA, 35 bp, Homo sapiens
  misc_binding (1)..(35): exon/intron Acceptor splice site gctggatgctccctgctccccacagGCATC GTGAA
gctggatgctccctgctccccacaggcatcgtgaa

SEQ ID NO: 60 - DNA, 18 bp, Homo sapiens
  exon (1)..(18): AIPL1 gene exon 1 Primer  5′ to 3′
ggacacctccctttctcc

SEQ ID NO: 61 - DNA, 18 bp, Homo sapiens
  exon (1)..(18): AIPL1 gene exon 1 Primer  5′ to  3′
gctggggctgcctggctg

SEQ ID NO: 62 - DNA, 20 bp, Homo sapiens
  exon (1)..(20): AIPL1 gene exon 2 Primer  5′ to 3′
gggccttgaacagtgtgtct

SEQ ID NO: 63 - DNA, 19 bp, Homo sapiens
  exon (1)..(19): AIPL1 gene exon 2 Primer  5′ to 3′
tttcccgaaacacagcagc

SEQ ID NO: 64 - DNA, 18 bp, Homo sapiens
  exon (1)..(18): AIPL1 gene exon 3 Primer  5′ to 3′
agtgagggagcaggattc

SEQ ID NO: 65 - DNA, 20 bp, Homo sapiens
  exon (1)..(20): AIPL1 gene exon 3 Primer  5′ to 3′
tgcccatgatgcccgctgtc

SEQ ID NO: 66 - DNA, 18 bp, Homo sapiens
  exon (1)..(18): AIPL1 gene exon 4 Primer 5′ to 3′
tttcgggtctctgatggg

SEQ ID NO: 67 - DNA, 17 bp, Homo sapiens
  exon (1)..(17): AIPL1 gene exon 4 Primer 5′ to 3′
gcaggctccccagagtc

SEQ ID NO: 68 - DNA, 19 bp, Homo sapiens
  exon (1)..(19): AIPL1 gene exon 5 Primer 5′ to 3′
gcagctgcctcaggtcatg

SEQ ID NO: 69 - DNA, 18 bp, Homo sapiens
  exon (1)..(18): AIPL1 gene exon 5 Primer 5′ to 3′
gtggggtggaaagaaaag

SEQ ID NO: 70 - DNA, 18 bp, Homo sapiens
  exon (1)..(18): AIPL1 gene exon 6 Primer 5′ to 3′
ctgggaagggagctgtag

SEQ ID NO: 71 - DNA, 19 bp, Homo sapiens
  exon (1)..(19): AIPL1 gene exon 6 Primer 5′ to 3′
aaaagtgacaccacgatcc

SEQ ID NO: 72 - protein, 384 aa, Homo sapiens
  PEPTIDE (1)..(384): Human AIPL1 Protein
MDAALLLNVEGVKKTILHGGTGELPNFITGSRVIFHFRTMKCDEERTVIDDSRQVGQPMHIIIGNMFKLEVWEILLTSMRVHEVAEFWCDTIHTGVYPILSRSLRQMAQGKDPTEWHVHTCGLANMFAYHTLGYEDLDELQKEPQPLVFVIELLQVDAPSDYQRETWNLSNHEKMKAVPVLHGEGNRLFKLGRYEEASSKYQEAIICLRNLQTKEKPWEVQWLKLEKMINTLILNYCQCLLKKEEYYEVLEHTSDILRHHPGIVKAYYVRARAHAEVWNEAEAKADLQKVLELEPSMQKAVRRELRLLENRMAEKQEEERLXCRNMLSQGATQPPAEPPTEPPAQSSTEPPAEPPTAPSAELSAGPPAEPATEPPPSPGHSLQH

SEQ ID NO: 73 - protein, 384 aa, Pan troglodytes
  PEPTIDE (1)..(384): Chimpanzee AIPL1 Protein
MDAALLLNVEGVKKTILHGGTGELPNFITGSRVIFHFRTMKCDEERTVIDDSRQVGQPMHIIIGNMFKLEVWEILLTSMRVHEVAEFWCDTIHTGVYPILSRSLRQMAQGKDPTEWHVHTCGLANMFAYHTLGYEDLDELQKEPQPLVFVIELLQVDAPSDYQRETWNLSNHEKMKAVPVLHGEGNRLFKLGRYEEASSKYQEAIICLRNLQTKEKPWEVQWLKLEKMINTLILNYCQCLLKKEEYYEVLEHTSDILRHHPGIVKAYYVRARAHAEVWNEAEAKADLRKVLELEPSMQKAVRRELRLLENRMAEKQEEERLRCRNMLSQGATQPPAEPPTEPPAQSSTEPPAEPPPAPSAELSAGPPAETATEPPPSPGHSLQH

SEQ ID NO: 74 - protein, 372 aa, Papio anubis
  PEPTIDE (1)..(372): Baboon AIPL1 Protein
MDAALLLNVEGVKKTILHGGTGELPNFITGSRVIFHFRTMKCDEERTVIDDSRQVDQPMHIIIGNMFKLEVWEILLTSMRVHEVAEFWCDTIHTGVYPILSRSLRQMAQGKDPTEWHVHTCGLANMFAYHTLGYEDLDELQKEPQPLIFVIELLQVDAPSDYQRETWNLSNHEKMKVVPVLHGEGNRLFKLGRYEEASSKYQEAIICLRNLQTKEKPWEVQWLKLEKMINTLTLNYCQCLLKKEEYYEVLEHTSDILRHHPGIVKAYYVRARAHAEVWNEAEAKADLQKVLELEPSMQKAVRRELRLLENRMAEKQEEERLRCRNMLSQGATQPPTEPPAEPHTAPPAELSTGPPAEPPAELPLSPGHSLQH

SEQ ID NO: 75 - protein, 328 aa, Bos taurus
  PEPTIDE (1)..(328): Cow AIPL1 Protein
MDATLLLNVEGIKKTILHGGTGDLPNFITGARVTFHFRTMKCDEERTVIDDSKQVGHPMHIIIGNMFKLEVWEILLTSMRVSEVAEFWCDTIHTGVYPILSRSLRQMAEGKDPTEWHVHTCGLANMFAYHTLGYEDLDELQKEPQPLIFIIELLQVEAPSQYQRETWNLNNQEKMQAVPILHGEGNRLFKLGRYEEASNKYQEAIVCLRNLQTKEKPWEVQWLKLEKMINTLILNYCQCLLKKEEYYEVLEHTSDILRHHPGIVKAYYVRARAHAEVWNEAEAKADLEKVLELEPSMRKAVQRELRLLENRLEEKREEERLRCRNMLG

SEQ ID NO: 76 - protein, 328 aa, Mus musculus
  PEPTIDE (1)..(328): Mouse AIPL1 Protein
MDVSLLLNVEGVKKTILHGGTGELPNFITGSRVTFHFRTMKCDEERTVIDDSKQVGQPMSIIIGNMFKLEVWETLLTSMRLGEVAEFWCDTIHTGVYPMLSRSLRQVAEGKDPTSWHVHTCGLANMFAYHTLGYEDLDELQKEPQPLVFLYELLQVEAPNEYQRETWNLNNEERMQAVPLLHGEGNRLYKLGRYDQAATKYQEAIVCLRNLQTKEKPWEVEWLKLEKMINTLILNYCQCLLKKEEYYEVLEHTSDILRHHPGIVKAYYMRARAHAEVWNAEEAKADLEKVLELEPSMRKAVLRELRLLESRLADKQEEERQRCRSMLG

SEQ ID NO: 77 - protein, 392 aa, Macaca mulatta
  PEPTIDE (1)..(392): Rhesus Monkey AIPL1 Protein
MDAALLLNVEGVKKTILHGGTGELPNFITGSRVIFHFRTMKCDEERTVIDDSRQVDQPMHIIIGNMFKLEVWEILLTSMRVHEVAEFWCDTIHTGVYPILSRSLRQMAQGKDPTEWHVHTCGLANMFAYHTLGYEDLDELQKEPQPLIFVIELLQVDAPSDYQRETWNLSNHEKMKVVPVLHGEGNRLFKLGRYEEASSKYQEAIICLRNLQTKEKPWEVQWLKLEKMINTLTLNYCQCLLKKEEYYEVLEHTSDILRHHPGIVKAYYVRARAHAEVWNEAEAKADLQKVLELEPSMQKAVRRELRLLENRMAEKQEEERLRCRNMLSQGATQPPAEPPAQPPTAPPAELSTGPPADPPAEPPTAPPAELSTGPPAEPPAELPLSPGHSLQH

SEQ ID NO: 78 - protein, 372 aa, Saimiri sciureus
  PEPTIDE (1)..(372): Squirrel Monkey AIPL1 Protein
MDAALLLNVEGVKKTILHGGTGELPNFITGSRVIFHFRTMKCDEERTVIDDSREVGQPMHIIIGNMFKLEVWEILLTSMRVREVAEFWCDTIHTGVYPILSRSLRQMAQGKDPTEWHVHTCGLANMFAYHTLGYEDLDELQKEPQPLIFVIELLQVDAPSDYQRETWNLSNHEKMKVVPVLHGEGNRLFKLGRYEEASSKYQEAIICLRNLQTKEKPWEVQWLKLEKMINTLILNYCQCLLKKEEYYEVLEHTSDILRHHPGIVKAYYVRARAHAEVWNEAEAKADLQKVLELEPSMQKAVRRELRLLENRMAEKQEEERLRCRNMLSQGATWSPAEPPAEPPAESSTEPPAEPPAEPPAELTLTPGHPLQH

(19) United States
(12) Patent Application Publication
(10) Pub. No.: US 2003/0022165 A1
(43) Pub. Date: Jan. 30, 2003

(54) Mutations in a novel photoreceptor-pineal gene on 17P cause leber congenital amaurosis (LCA4)

(76) Inventors: Melanie M. Sohocki, Houston, TX (US); Stephen P. Daiger, Houston, TX (US)

Correspondence Address: ROBERT W STROZIER, PLLC, 2925 BRIARPARK, SUITE 930, HOUSTON, TX, 77042, US

(21) Appl. No.: 09/765,061

(22) Filed: Jan. 17, 2001

(51) Int. Cl. C12Q 1/68; C07H 21/04

(52) U.S. Cl. 435/6; 536/24.3

(57) ABSTRACT

A novel photoreceptor/pineal-expressed gene encoding aryl-hydrocarbon receptor interacting protein-like 1 (AIPL1), the associated protein like amino acid sequence and methods for identifying the presence of the sequence in patients. Leber congenital amaurosis (LCA) is the most severe form of inherited retinal dystrophy and the most frequent cause of inherited blindness in children. LCA is usually inherited in an autosomal recessive fashion, although rare dominant cases have been reported. One form of LCA, LCA4, maps to chromosome 17p13 and is genetically distinct from other forms of LCA. The inventors recently identified the gene associated with LCA4, AIPL1 (aryl-hydrocarbon receptor interacting protein-like 1) and identified three mutations that were the cause of blindness in five families with LCA.

### GOVERNMENTAL SUPPORT

The invention disclosed herein was developed impart from funds from grant EY07142 from the National Eye Institute-National Institutes of Health.

### RELATED APPLICATION

This application claims priority to United States Provisional Application set bearing Express Mail Label EL 389 348 319 US to the United States Patent and Trademark Office on Jan. 4, 2001.

### BACKGROUND OF THE INVENTION

1. Field of the Invention

The present invention relates to a novel a novel photoreceptor/pineal-expressed gene encoding aryl-hydrocarbon receptor interacting protein-like 1 (AIPL1).

More particularly, the present invention relates to a DNA sequence for encoding AIPL1 and its mutants, DNA anti-sense probes including a sequence of bases including a mutation of the AIPL1 gene, synthetic protein made from a DNA sequence encoding AIPL1, transfection vehicles including a DNA sequence for encoding AIPL1, methods for transfecting retinal cells transiently or permanently, method for diagnosing retinal diseases associated with AIPL1 mutations, methods for treating retinal diseases by administering a transfection vehicle including a DNA sequence for encoding AIPL1 to a retinal site, methods for detecting specific mutations in a patient population and methods for treating retinal diseases by administering synthetic wild-type AIPL1 alone or in combination with other proteins or transfection vehicles encoding wild-type AIPL1 and/or other wild-type proteins.

2. Description of the Related Art

Leber congenital amaurosis (LCA, MIM 204000) accounts for at least 5% of all inherited retinal disease (Kaplan J., Bonneau D., Frezal J., Munnich A. & Dufier J. L. Clinical and genetic heterogeneity in retinitis pigmentosa. Hum. Genet. 85, 635-642 (1990)), and is the most severe inherited retinopathy, with the earliest age of onset (Foxman, S. G., Heckenlively, J. R., Batemen, B. J. & Wirstschafter, J. D. Classification of congenital and early-onset retinitis pigmentosa. Arch. Ophthalmol. 103, 1502-1507 (1985)). LCA is diagnosed at birth or in the first few months of life, with severely impaired vision or blindness, nystagmus, and a markedly abnormal or flat electroretinogram (ERG). Mutations in GUCY2D (Perrault, I. et al. Retinal-specific guanylate cyclase gene mutations in Leber's congenital amaurosis. Nature Genet. 14, 461-464 (1996)), RPE65 (Marlhens, F. et al. Mutations in RPE65 cause Leber's congenital amaurosis. Nature Genet. 17, 139-141 (1997)) and CRX (Freund, C. L. et al. De novo mutations in the CRX homeobox gene associated with Leber congenital amaurosis. Nature Genet. 18, 311-312 (1998)) are known to cause LCA. However, one study identified disease-causing GUCY2D mutations in only 8 of 15 families whose LCA locus maps to 17p13.1 (Perrault, I. et al. Retinal-specific guanylate cyclase gene mutations in Leber's congenital amaurosis. Nature Genet. 14, 461-464 (1996)), suggesting another LCA locus might be located on 17p13.1. Confirming this prediction, the LCA in one Pakistani family mapped to 17p13.1, between D17S849 and D17S960—a region that excludes GUCY2D. The LCA in this family has been designated LCA4 (Hameed, A. et al. A novel locus for Leber congenital amaurosis with anterior keratoconus mapping to 17p13. Invest. Ophthalmol. Vis. Sci. in press (1999)).

### SUMMARY OF THE INVENTION

The present invention provides a new photoreceptor/pineal gene or DNA sequence, aryl-hydrocarbon receptor interacting protein-like 1 (AIPL1), encoding an aryl-hydrocarbon receptor interacting protein, which maps within an LCA4 candidate region of chromosome 17p13. The protein comprises three tetratricopeptide (TPR) motifs, which are thought to impart it with nuclear transport or chaperone activity to the protein.

The present invention also provides gene sequences encoding mutant forms of the AIPL1 gene, where the mutants forms are selected from the group consisting of Ala336Δ2, Trp278X, Cys239Arg, M79T, L88X, V96I, T124I, P376S, Q163X, A197P, IVS2-2, G262S, R302L, P351D12, Cys42X (TGT→TGA), Val33ins 8 bp (GTGATCTT), Leu257del 9 bp (CTCCGGCAC) or mixtures or combinations thereof.

The present invention provides a method for identifying photoreceptor/pineal-expressed gene, aryl-hydrocarbon receptor interacting protein-like 1 (AIPL1) including specific mutations that give rise to LCA or other retinal diseases.

The present invention also provides an anti-sense base sequence capable of binding to and allowing identification of a mutant AIPL1 gene including anti-sense sequences for the mutants selected from the group consisting of Ala336Δ2, Trp278X, Cys239Arg, M79T, L88X, V96I, T124I, P376S, Q163X, A197P, IVS2-2, G262S, R302L, P351D12, Cys42X (TGT→TGA), Val33ins 8 bp (GTGATCTT), Leu257del 9 bp (CTCCGGCAC) or mixtures or combinations thereof.

The present invention also provides chip-based probes including a chip surface have attached thereto DNA anti-sense sequences having a length between about 4 and about 35 base units, where each anti-sense sequence comprises a mutation of the AIPL1 gene including the mutations selected from the group consisting of Ala336Δ2, Trp278X, Cys239Arg, M79T, L88X, V96I, T124I, P376S, Q163X, A197P, IVS2-2, G262S, R302L, P351D12, Cys42X (TGT→TGA), Val33ins 8 bp (GTGATCTT), Leu257del 9 bp (CTCCGGCAC) or mixtures or combinations thereof.

The present invention also provides a library of anti-sense DNA probes, where each probe is an anti-sense DNA sequence comprising a mutation of the AIPL1 gene including the mutations selected from the group consisting of Ala336Δ2, Trp278X, Cys239Arg, M79T, L88X, V96I, T124I, P376S, Q163X, A197P, IVS2-2, G262S, R302L, P351D12, Cys42X (TGT→TGA), Val33ins 8 bp (GTGATCTT), Leu257del 9 bp (CTCCGGCAC) or mixtures or combinations thereof.

The present invention also provides a method for screening patients including the steps of fragmenting a DNA sample from a patient into fragments of single stranded DNA, contacting the fragments with an anti-sense probe including a mutation of the AIPL1 gene including the mutations selected from the group consisting of Ala336Δ2, Trp278X, Cys239Arg, M79T, L88X, V96I, T124I, P376S, Q163X, A197P, IVS2-2, G262S, R302L, P351D12, Cys42X (TGT→TGA), Val33ins 8 bp (GTGATCTT), Leu257del 9 bp (CTCCGGCAC) or mixtures or combinations thereof and detecting any duplexed fragments where the duplex is formed between a fragment and the probe.

The present invention also provides a method for screening patients including the steps of fragmenting a DNA sample from a patient into fragments of single stranded DNA, contacting the fragments with an anti-sense probe comprising a mutation of the AIPL1 gene including the mutations selected from the group consisting of Ala336Δ2, Trp278X, Cys239Arg, M79T, L88X, V96I, T124I, P376S, Q163X, A197P, IVS2-2, G262R302L, P351D12, Cys42X (TGT→TGA), Val33ins 8 bp (GTGATCTT), Leu257del 9 bp (CTCCGGCAC) or mixtures or combinations thereof, amplifying any duplexed DNA, and detecting the duplexed DNA where the duplexed DNA is formed between a fragment and the probe.

The present invention also provides a method for screening patients including the steps of fragmenting a DNA sample from a patient into fragments of single stranded DNA, contacting the fragments with an anti-sense probe including a mutation of the AIPL1 gene including the mutations selected from the group consisting of Ala336Δ2, Trp278X, Cys239Arg, M79T, L88X, V96I, T124I, P376S, Q163X, A197P, IVS2-2, G262S, R302L, P351D12, Cys42X (TGT→TGA), Val33ins 8 bp (GTGATCTT), Leu257del 9 bp (CTCCGGCAC) or mixtures or combinations thereof, separating any formed DNA duplexes, amplifying the duplexed DNA, and detecting the duplexed DNA where the duplex is formed between a fragment and the probe.

The present invention also provides a method for ameliorating at least one symptom of a retinal disease including administering to a retinal site an effective amount of a protein prepared from a DNA coding sequence encoding a wild-type AIPL1 gene. The administration can by oral administration, intravenous administration intra-arterial administration, site specific administration, other similar mean of administering a gene sequence or protein or mixtures or combinations thereof.

The present invention also provides a method for ameliorating at least one symptom of a retinal disease including administering to cells of a retinal site an expression vector including a wild-type AIPL1 coding sequence to cause the expression of a protein corresponding to the AIPL1 coding sequence, where the protein ameliorates at least on symptom of a retinal disease.

The present invention also provides a method for identifying patients with mutations to an AIPL 1 gene including the step of obtaining a DNA sample from the patient, isolating polynucleotide extracted from said sample, hybridizing a detectably labeled oligonucleotide to the isolated polynucleotide, the oligonucleotide having at its 3′ end at least 15 nucleotides complementary to a wild type polynucleotide sequence having at least one mutation or polymorphism, attempting to extend the oligonucleotide at its 3′-end; ascertaining the presence or absence of a detectably labeled extended oligonucleotide; and correlating the presence or absence of a detectably labeled extended oligonucleotide in step (e) with the presence or absence of a AIPL1 mutation. The mutation are selected from the group consisting of Ala336Δ2, Trp278X, Cys239Arg, M79T, L88X, V96I, T124, P376S, Q163X, A197P, IVS2-2, G262S, R302L, P351D12, Cys42X (TGT→TGA), Val33ins 8 bp (GTGATCTT), Leu257del 9bp (CTCCGGCAC) or mixtures or combinations thereof; while the benign polymorphisms are selected from the group consisting of IVS1-9G→A, IVS2+66G→C, IVS2-88C→T, IVS2-14G→A, IVS2-10A→C, IVS3-25T→C, IVS3-21T→C, IVS5+18G→A, Asp90His, Phe37Phe, Ser78Ser, Cys89Cys, Leu100Leu, His172His, Pro217Pro, Asp255Asp and mixtures and combinations thereof.

### DESCRIPTION OF THE DRAWINGS

The invention can be better understood with reference to the following detailed description together with the appended illustrative drawings in which like elements are numbered the same:

FIG. 1 depicts a gene and protein structure of AIPL1; a. AIPL1 consists of six exons, with alternate polyadenylation sites in the 3′ untranslated region, shown by arrows. Cys239Arg denotes the location of the TGC→CGC missense mutation in exon 5 of the RFS128 family. Trp278X denotes the location of the TGG→TGA nonsense mutation in exon 6 of the KC, MD, RFS127 and RFS121 families. Ala336Δ2 denotes the location of the 2 bp deletion in exon 6 of RFS121. Benign coding sequence substitutions identified were Phe37Phe (TTT/TTC; 0.98/0.02 frequency), Cys89Cys (TGC/TGT; 0.99/0.01), Asp90His (GAC/CAC; 0.84/0.16), Leu100Leu (CTG/CTA; 0.57/0.43) and Pro217Pro (CCG/CCA; 0.61/0.39) b. Protein sequence of AIPL1. The alignment demonstrates the high level of sequence conservation between rat and human AIPL1, and mouse and human AIP. Identical residues in the four sequences are noted with an asterisk; identical residues in three of the sequences are indicated with a period.;

FIG. 2 depicts a fluorescence in situ hybridization (FISH); AIPL1-containing bacterial artificial chromosome (BAC), shown in red, hybridizes to 17p13.1, consistent with placement of AIPL1 in the Stanford G3 radiation hybrid panel. These data refute the original placement of AIPL1 to 17p13.3 by placement in the GeneBridge 4.0 radiation hybrid panel. Chromosome 17 alpha-satellite DNA is indicated in green.;

FIG. 3 depicts an expression of AIPL1 in human tissues; Northern blots from adult tissues were incubated with an AIPL1 probe. Total retinal RNA blot, exposed 4 hours at −70° C. (upper left) and polyA⁺ RNA multi-tissue Northern (MTN), exposed 72 hours at −70° C. (upper right); No signal was observed in MTN at 4, 24, or 48-hour exposure. Lane 1, adult retina; lane 2, heart; lane 3, whole brain; lane 4, placenta; lane 5, lung; lane 6, liver; lane 7, skeletal muscle; lane 8, kidney; lane 9, pancreas. Both blots were incubated with a β-actin probe as a control (lower panel). Solid arrows indicate mRNA molecules of the predicted sizes, 1538 and 2247 bp, in retina;

FIG. 4 depicts a retina and pineal expression of Aipl1; a. Digoxygenin in situ hybridization of Aipl1 in adult mouse retina, with expression throughout the outer nuclear layer and photoreceptor inner segments. Color reaction time is 4 days. b. Sense control of “a” with same reaction time. A slight background signal is observed across photoreceptor outer segments. c. Short (16 hour) color reaction of Aipl1 in adult mouse retina, showing a high level of mRNA in photoreceptor inner segments. d. Expression of Aipl1 in adult mouse pineal. Color reaction time is 4 days. e. Sense control of “d”, with same reaction time. f. Expression of Aipl1 mRNA in P14 rat pineal. Color reaction time is 4 days. g. Sense control of “f”, with same reaction time. Scale bar for a-c is 30 μm, for d and e is 50 μm, and f and g is 70 μm. RPE-retinal pigment epithelium, OS-outer photoreceptor segment, IS-inner photoreceptor segment, ONL-outer nuclear layer, INL-inner nuclear layer, GCL-ganglion cell layer. Immunolocalization of the AIPL1 protein has not been performed; therefore, site of AIPL1 protein localization is currently unknown;

FIG. 5 depicts a pedigrees and mutation screen of AIPL1 in families; a. The Trp278X mutation is homozygous in three families: KC, MD and RFS127. SSCA of all living individuals of the KC pedigree demonstrate segregation of the mutant allele. Top electropherogram: an unaffected control (TGG/TGG). Middle: heterozygous G/A mutation at codon 278. Bottom: DNA sequence of a homozygous, affected member of MD (TGA/TGA). b. The RFS121 affected individuals are compound heterozygotes for the Trp278X and Ala336Δ2 bp mutations. Top electropherogram: unaffected control, bottom: heterozygous G/A mutation at codon 278 (left) and heterozygous 2 bp deletion beginning in codon 336 (right) in an affected individual of RFS121.c. The Cys239Arg mutation found in family RFS128. Top electropherogram: unaffected control (TGC/TGC), bottom: DNA sequence of a homozygous, affected individual (CGC/CGC);

FIG. 6 depicts a fundus photograph of affected LCA patient (eleven years of age), displaying typical symptoms of Leber congenital amaurosis; widespread retinal pigment epithelium changes with pigment clumping, attenuated retinal vessels, pale optic disk, and macular atrophy are evident. Members of the KC family also display keratoconus; because AIPL1 is not expressed in the cornea, it is possible that this symptom is secondary to LCA in this family, due to eye rubbing, etc;

FIG. 7 depicts pedigrees of four LCA families with the W278X mutation, with representative electropherogram. Mutant sequence is listed above and wild-type sequence (in italics) below each electropherogram. (A) Pedigrees for families HEM6 and HEM109, whose affected probands are homozygous for the Trp278X mutation. (B) Family HEM24, whose affected individuals are compound heterozygotes of Trp278X (left) and a splice-site mutation, IVS2-2A>G. (C) Family JH2873, whose affected proband is a compound heterozygote for the Trp278X mutation and G262S (GGC→AGC). The nucleotide substitution of the G262S mutation occurs at the last base of exon 5;

FIG. 8 depicts pedigrees of seven LCA families with previously unreported AIPL1 mutations. (A) Family JH3749. Affected individuals are homozygous for M79T, ATG→ACG. (B) Family UCL01. Affected individuals are homozygous for W88X, TGG→TGA. (C) Family HEM115, whose affected proband is heterozygous for V96I, GTC→ATC, in a highly conserved residue. (D) Family JH1379, whose affected proband is a compound heterozygote for T1241, ACA→ATA, and P376S, CCG→TCG. (E) Affected individuals of family JH3285 are homozygous for Q163X, CAG→TAG. (F) Affected members of family HEM26 are homozygous for A197P, GCC→CCC. (G) The affected proband of JH3860 is homozygous for R302L, CGC→CTC;

FIG. 9 depicts pedigrees of two families with probands heterozygous for the 12 bp AIPL1 deletion, and representative electropherogram. The mutant allele was sucloned and sequenced to confirm size and sequence of deletion. Family members who have not been clinically examined, and, therefore, are of unknown phenotype are designated by an “?” within the symbol. (A) UTAD231, with the original diagnosis of “cone-rod dystrophy, possibly dominant”. Two unaffected individuals in this family lack the 12 bp deletion. (B) UTAD907, whose original diagnosis was “juvenile RP, possibly dominant”; and

FIG. 10 depicts disease-associated mutations within AIPL1.

### DEFINITIONS

Unless otherwise stated, the following terms shall have the following meanings:

The term “primer” denotes a specific oligonucleotide sequence that is complementary to a target nucleotide sequence and used to hybridize to the target nucleotide sequence. A primer serves as an initiation point for nucleotide polymerization catalyzed by either DNA polymerase, RNA polymerase or reverse transcriptase.

The term “probe” denotes a defined nucleic acid segment (or nucleotide analog segment, e.g., PNA as defined hereinbelow) which can be used to identify a specific polynucleotide present in samples bearing the complementary sequence.

The term “polynucleotide” as used herein means a polymeric form of nucleotides of any length, either ribonucleotides or deoxyribonucleotides. This term refers only to the primary structure of the molecule. Thus, the term includes double- and single-stranded DNA, as well as double- and single-stranded RNA. It also includes modifications, such as methylation or capping and unmodified forms of the polynucleotide. The terms “polynucleotide,” “oligomer,” “oligonucleotide,” and “oligo” are used interchangeably herein.

The term “isolated” means that the material is removed from its original environment (e.g., the natural environment if it is naturally occurring). For example, a naturally occurring polynucleotide or polypeptide present in a living animal is not isolated, but the same polynucleotide or DNA or polypeptide that is separated from some or all of the coexisting materials in the natural system, is isolated. Such polynucleotide could be part of a vector and/or such polynucleotide or polypeptide could be part of a composition, and still be isolated in that the vector or composition is not part of its natural environment.

“Recombinant host cells,” “host cells,” “cells,” “cell lines,” “cell cultures,” and other such terms denoting microorganisms or higher eukaryotic cell lines cultured as unicellular entities refer to cells that can be, or have been, used as recipients for recombinant vector or other transferred DNA, and include the original progeny of the original cell that has been transfected.

As used herein the term “mutation” means any change in DNA sequence from the wild-type or consensus sequence, including, but not limited to, pathogenic changes.

As used herein “replicon” means any genetic element, such as a plasmid, a chromosome or a virus, that behaves as an autonomous unit of polynucleotide replication within a cell.

The term “animal” is defined as a member of the animal kingdom including mammals and humans.

The term “mammal” is defined as any class of warm-blooded higher vertebrates that includes humans.

A “vector” is a replicon in which another polynucleotide segment is attached, such as to bring about the replication and/or expression of the attached segment.

The term “control sequence” refers to a polynucleotide sequence that is necessary to effect the expression of a coding sequence to which it is ligated. The nature of such control sequences differs depending upon the host organism. In prokaryotes, such control sequences generally include a promoter, a ribosomal binding site and terminators; in eukaryotes, such control sequences generally include promoters, terminators and, in some instances, enhancers. The term “control sequence” thus is intended to include at a minimum all components whose presence is necessary for expression, and also may include additional components whose presence is advantageous, for example, leader sequences.

“Operably linked” refers to a situation wherein the components described are in a relationship permitting them to function in their intended manner. Thus, for example, a control sequence “operably linked” to a coding sequence is ligated in such a manner that expression of the coding sequence is achieved under conditions compatible with the control sequence.

The term “open reading frame” or “ORF” refers to a region of a polynucleotide sequence that encodes a polypeptide. This region may represent a portion of a coding sequence or a total coding sequence.

A “coding sequence” is a polynucleotide sequence that is transcribed into MRNA and translated into a polypeptide when placed under the control of appropriate regulatory sequences. The boundaries of the coding sequence are determined by a translation start codon at the 5′-terminus and a translation stop codon at the 3′-terminus. A coding sequence can include, but is not limited to, mRNA, cDNA and recombinant polynucleotide sequences.

As used herein, “epitope” means an antigenic determinant of a polypeptide or protein. Conceivably, an epitope can comprise three amino acids in a spatial conformation that is unique to the epitope. Generally, an epitope consists of at least five such amino acids and more usually, it consists of at least eight to ten amino acids. Methods of examining spatial conformation are known in the art and include, for example, x-ray crystallography and two-dimensional nuclear magnetic resonance.

A “conformational epitope” is an epitope that is comprised of a specific juxtaposition of amino acids in an immunologically recognizable structure, such amino acids being present on the same polypeptide in a contiguous or non-contiguous order or present on different polypeptides.

A polypeptide is “immunologically reactive” with an antibody when it binds to an antibody due to antibody recognition of a specific epitope contained within the polypeptide. Immunological reactivity may be determined by antibody binding, more particularly, by the kinetics of antibody binding, and/or by competition in binding using as competitor(s) a known polypeptide(s) containing an epitope against which the antibody is directed. The methods for determining whether a polypeptide is immunologically reactive with an antibody are known in the art.

As used herein, the term “immunogenic polypeptide containing an epitope of interest” means naturally occurring polypeptides of interest or fragments thereof, as well as polypeptides prepared by other means, for example, by chemical synthesis or the expression of the polypeptide in a recombinant organism.

“Wild-type” is described as the genotype that naturally occurs in the normal population.

“Analog” is defined as a compound that resembles another in function.

The term “test sample” refers to a component of an individual's body that is the source of the analyte (such as antibodies of interest or antigens of interest). These components are well known in the art. A test sample is typically anything suspected of containing a target sequence. Test samples can be prepared using methodologies well known in the art such as by obtaining a specimen from an individual and, if necessary, disrupting any cells contained thereby to release target nucleic acids. These test samples include biological samples that can be tested by the methods of the present invention described herein and include human and animal body fluids such as whole blood, serum, plasma, cerebrospinal fluid, sputum, bronchial washing, bronchial aspirates, urine, lymph fluids, and various external secretions of the respiratory, intestinal and genitourinary tracts, tears, saliva, milk, white blood cells, myelomas and the like; biological fluids such as cell culture supernatants; tissue specimens that may be fixed; and cell specimens that may be fixed.

### DETAILED DESCRIPTION OF THE INVENTION

The inventors have found that a gene located within an LCA4 candidate region of chromosome 17p13 and various of its mutation are involved in LCA and DNA antisense probes designed therefrom can be constructed to screen patients for potential vision threatening diseases including LCA. The gene is generally referred to herein as the AIPL1 gene. The inventors have also found that proteins resulting from the expression of the wild-type or mutant forms of the gene may be formulated or expressed in patients to ameliorate disease symptoms for LCA and other retinal disorders. The inventors have also identified a homozygous nonsense mutation at codon 278 which is present in all affected members of the original LCA4 family. AIPL1 mutations may cause approximately 20% of recessive LCA, as disease-causing mutations were identified in 3 of 14 LCA families not tested previously for linkage.

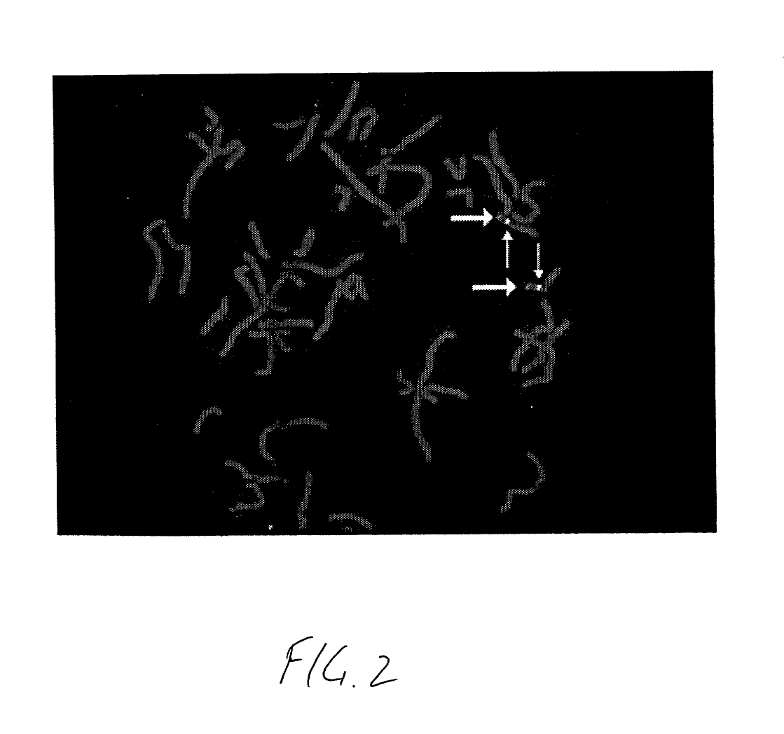

Previously, the inventors found that STSs designed to the retina/pineal-expressed EST clusters THC220430 and THC90422 originally mapped to 17p13 (Sohocki, M. M., Malone, K. A., Sullivan, L. S. & Daiger, S. P. Localization of retina/pineal—expressed sequences (ESTs): identification of novel candidate genes for inherited retinal disorders. Genomics 58, 29-33 (1999)), near a retinitis pigmentosa (RP13) candidate region (Greenberg, J., Goliath, R., Beighton, P., & Ramesar, R. A new locus for autosomal dominant retinitis pigmentosa on the short arm of chromosome 17. Hum Mol Genet 3, 915-918 (1994)). Further testing refined the localization to 17p13.1, between SHGC-2251 and SHGC-6095, within an LCA4 candidate region, and approximately 2.5 megabases (Mb) distal to GUCY2D. Fluorescence in situ hybridization described more fully herein and shown in FIG. 2 confirmed the localization. AIPL1-containing bacterial artificial chromosome (BAC) hybridizes to 17p13.1, consistent with placement of AIPL1 in the Stanford G3 radiation hybrid panel. These data refute the original placement of AIPL1 to 17p13.3 by placement in the GeneBridge 4.0 radiation hybrid panel. Chromosome 17 alpha-satellite DNA is indicated in green.

AIPL1 was screened for mutations in 512 unrelated probands with a range of retinal degenerative diseases to determine if AIPL1 mutations cause other forms of inherited retinal degeneration and to determine the relative contribution of AIPL1 mutations to inherited retinal disorders in populations worldwide. The inventors identified 11 LCA families whose retinal disorder is caused by homozygous or compound heterozygous AIPL1 mutations. The inventors also identified affected individuals in two apparently dominant families, diagnosed with juvenile retinitis pigmentosa (RP), or dominant cone-rod dystrophy, respectively, who are heterozygous for a 12 base-pair AIPL1 deletion. Our results suggest that AIPL1 mutations cause approximately 7% of LCA worldwide and may cause dominant retinopathy.

The present invention also relates to the identification of certain retinal diseases and disorders including LCA, juvenile retinitis pigmentosa (RP), dominant cone-rod dystrophy, and other inherited and/or acquired retinopathies. The present invention can be used as a diagnosis and/or treatment of inherited and/or acquired retinopathies in animals including humans and/or development of animal models for diseases caused by or related to mutation in AIPL1.

Leber congenital amaurosis (LCA, Mendelian Inheritance in Man (MIM) No.204000) accounts for approximately 5% of all inherited retinal disease (Kaplan J, Bonneau D, Frezal J, Munnich A, Dufier J L. Clinical and genetic heterogeneity in retinitis pigmentosa. Hum Genet 85:635-642, 1990), and is the most severe, with the earliest age of onset (Foxman S G, Heckenlively J R, Batemen B, Wirstschafter J D. Classification of congenital and early-onset retinitis pigmentosa. Arc. Ophthalmol 103:1502-1507, 1985). LCA is genetically heterogeneous, and until recently, only three genes associated with LCA had been identified: GUCY2D (Perrault I, Rozet J M, Calvas P, Gerber S, Camuzat A, Dollfus H, Chatelin S, Souied E, Ghazi I, Leowski C, Bonnemaison M, Paslier D L, Frezal J, Dufier J, Pittler S, Munnich A, Kaplan J. Retinal-specific guanylate cyclase gene mutations in Leber's congenital amaurosis. Nature Genet 14:461-464, 1996), CRX (Freund C, Wang Q L, Chen S, Muskat B L, Wiles C D, Sheffield V C, Jacobson S G, McInnes R R, Zack, D J, Stone E M. De novo mutations in the CRX homeobox gene associated with Leber congenital amaurosis. Nature Genet 18:311-312, 1998), and RPE65 (Marlhens F, Bareil C, Griffoin J M, Zrenner E, Amalric P, Eliaou C, Liu S Y, Harris E, Redmond T M, Arnaud B, Claustres M, Hamel C P. Mutations in RPE65 cause Leber's congenital amaurosis. Nature Genet 17:139-141, 1997). The inventors have isolated an aryl hydrocarbon-interacting protein-like 1 (AIPL1) which maps within 2.5 Megabases (Mb) of GUCY2D on 17p13 and is the fourth gene to be associated with LCA.

The present invention provides a new photoreceptor/pineal gene or DNA sequence, aryl-hydrocarbon receptor interacting protein-like 1 (AIPL1), encoding an aryl-hydrocarbon receptor interacting protein, which maps within an LCA4 candidate region of chromosome 17p13. The protein comprises three tetratricopeptide (TPR) motifs, which are thought to impart it with nuclear transport or chaperone activity to the protein.

The present invention also provides gene sequences encoding mutant forms of the AIPL1 gene selected from the group consisting of Ala336Δ2, Trp278X, Cys239Arg, M79T, L88X, V96I, T124I, P376S, Q163X, A197P, IVS2-2, G262S, R302L, P351D12, Cys42X (TGT→TGA), Val33ins 8 bp (GTGATCTT), Leu257del9 bp (CTCCGGCAC) or mixtures or combinations thereof.

The present invention provides a method for identifying photoreceptor/pineal-expressed gene, aryl-hydrocarbon receptor interacting protein-like 1 (AIPL1) including specific mutations that give rise to LCA or other retinal diseases.

The present invention also provides a anti-sense base sequence capable of binding to and allow identification of a mutant aipl1 gene including the mutants selected from the group consisting of Ala336Δ2, Trp278X, Cys239Arg, M79T, L88X, V96I, T124I, P376S, Q163X, A197P, IVS2-2, G262S, R302L, P351D12, Cys42X (TGT→TGA), Val33ins 8 bp (GTGATCTT), Leu257del 9 bp (CTCCGGCAC) or mixtures or combinations thereof.

The present invention also provides chip-based probes including a chip surface have attached thereto DNA anti-sense sequences having a length between about 4 and about 35 base units, where each sequence comprises a given set of bases including a mutation of the AIPL1 gene including a mutation selected from the group consisting of Ala336Δ2,Trp278X, Cys239Arg, M79T, L88X, V96, T124I, P376S, Q163X, A197P, IVS2-2, G262S, R302L, P351D12 or mixtures or combinations thereof.

The present invention also provides a library of DNA probes, where each probe is a DNA sequence including a mutation of the AIPL1 gene including the mutations selected from the group consisting of Ala336Δ2, Trp278X, Cys239Arg, M79T, L88X, V96I, T124I, P376S, Q163X, A197P, IVS2-2, G262S, R302L, P351D12, Cys42X (TGT→TGA), Val33ins 8 bp (GTGATCTT), Leu257del 9 bp (CTCCGGCAC) or mixtures or combinations thereof.

The present invention also provide a method for screening patients including the steps of fragmenting a DNA sample from a patient into fragments of single stranded DNA, contacting the fragments with an anti-sense probe including a mutation of the AIPL1 gene including the mutations selected from the group consisting of Ala336Δ2, Trp278X, Cys239Arg, M79T, L88X, V96I, T124I, P376S, Q163X, A197P, IVS2-2, G262S, R302L, P351D12, Cys42X (TGT→TGA), Val33ins 8 bp (GTGATCTT), Leu257del 9 bp (CTCCGGCAC) or mixtures or combinations thereof and detecting any duplexed fragments where the duplex is formed between a fragment and the probe.

The present invention also provide a method for screening patients including the steps of fragmenting a DNA sample from a patient into fragments of single stranded DNA, contacting the fragments with an anti-sense probe including a mutation of the AIPL1 gene including the mutations selected from the group consisting of Ala336Δ2, Trp278X, Cys239Arg, M79T, L88X, V96I, T124I, P376S, Q163X, A197P, IVS2-2, G262S, R302L, P351D12, Cys42X (TGT→TGA), Val33ins 8 bp (GTGATCTT), Leu257del 9 bp (CTCCGGCAC) or mixtures or combinations thereof, amplifying any duplexed DNA, and detecting the duplexed DNA where the duplexed DNA is formed between a fragment and the probe.

The present invention also provide a method for screening patients including the steps of fragmenting a DNA sample from a patient into fragments of single stranded DNA, contacting the fragments with an anti-sense probe including a mutation of the AIPL1 gene including the mutations selected from the group consisting of Ala336Δ2, Trp278X, Cys239Arg, M79T, L88X, V96I, T124I, P376S, Q163X, A197P, IVS2-2, G262S, R302L, P351D12, Cys42X (TGT→TGA), Val33ins 8 bp (GTGATCTT), Leu257del 9 bp (CTCCGGCAC) or mixtures or combinations thereof, separating any formed DNA duplexes, amplifying the duplexed DNA, and detecting the duplexed DNA where the duplex is formed between a fragment and the probe.

The present invention also provides a method for ameliorating at least one symptom of a retinal disease including administering to a retinal site an effective amount of a protein prepared from a DNA coding sequence encoding a wild-type AIPL1 gene.

The present invention also provides a method for ameliorating at least one symptom of a retinal disease including administering to cells of a retinal set an expression vector including a wild-type AIPL1 coding sequence to cause the express of a protein corresponding to the AIPL1 coding sequence, where the protein ameliorates at least on symptom of a retinal disease.

The present invention also provides a method for determining the presence or absence of a AIPL1 mutation which contributes to a retinal disease or disorder including hybridizing an oligonucleotide to nucleic acid from the patient sample, wherein said oligonucleotide is complementary to an AIPL1 encoding sequence having at least one mutation; and detecting hybridization between the oligonucleotide and the nucleic acid, the identity of the nucleotide indicating whether the sample has an AIPL1 mutation or are sensitive to drugs which treat retinal diseases. The oligonucleotide can be mobile or immobilized to a solid support and the sample nucleic acid can be labeled or unlabeled and the oligonucleotide can be labeled or unlabeled; preferably, either the nucleic acid or oligonucleotide is labeled to facilitate detection.

The present invention also provides a method of screening a patient for the presence of an AIPL1 mutation including obtaining a sample from the patient, detecting one or more substitutions in an AIPL1 polypeptide from the patient sample, and correlating the one or more substitutions with a retinal disease or a propensity of passing a retinal disease to offspring. The substitutions include any of the substitutions set forth herein.

The present invention also provides a method of determining an AIPL1 mutation including the steps of detecting a mutation in a nucleic acid encoding AIPL1 protein in a sample, the mutation comprising one or more mutations selected from the group consisting of Ala336Δ2, Trp278X, Cys239Arg, M79T, L88X, V96I, T124I, P376S, Q163X, A197P, IVS2-2, G262S, R302L, P351D12, Cys42X (TGT→TGA), Val33ins 8 bp (GTGATCTT), Leu257del 9 bp (CTCCGGCAC) or mixtures or combinations thereof; and correlating detection of the mutation with a retinal disease or a propensity of pass a retinal disease to offspring.

The present invention also provides an isolated AIPL1 amino acid sequence comprising an amino acid sequence having at least one mutation, said mutation selected from the group consisting of Ala336Δ2, Trp278X, Cys239Arg, M79T, L88X, V96I, T124I, P376S, Q163X, A197P, IVS2-2, G262S, R302L, P351D12, Cys42X (TGT→TGA), Val33ins 8 bp (GTGATCTT), Leu257del 9 bp (CTCCGGCAC) or mixtures or combinations thereof.

The present invention also provides an isolated nucleic acid sequence, wherein the nucleic acid encodes at least one AIPL1 mutation resulting in an amino acid substitution at any one of the following locations 336, 278, 239, 79, 88, 96, 124, 376, 163, 197, IVS2-2, 262, 302, 351D12, 42, 33 ins 8 bp (GTGATCTT), 257del 9 bp (CTCCGGCAC) or mixtures or combinations thereof.

The present invention also provides an isolated cell, the cell having at least one substitution in the cell's AIPL1 encoding region.

The present invention also provides a method to determine if a patient has an AIPL1 mutation comprising obtaining a patient sample; determining if patient's AIPL1 has at least one substitution; and correlating this substitution with the patient's retinal disease state or with patient's propensity to pass a retinal disease to offspring.

The present invention provides assays that utilize specific binding members. A “specific binding member,” as used herein, is a member of a specific binding pair. That is, two different molecules where one of the molecules, through chemical or physical means, specifically binds to the second molecule. Therefore, in addition to antigen and antibody specific binding pairs of common immunoassays, other specific binding pairs can include biotin and avidin, carbohydrates and lectins, complementary nucleotide sequences, effector and receptor molecules, cofactors and enzymes, enzyme inhibitors, and enzymes and the like. Furthermore, specific binding pairs can include members that are analogs of the original specific binding members, for example, an analyte-analog. Immunoreactive specific binding members include antigens, antigen fragments, antibodies and antibody fragments, both monoclonal and polyclonal and complexes thereof, including those formed by recombinant DNA molecules.

Specific binding members include “specific binding molecules.” A specific binding molecule” intends any specific binding member, particularly an immunoreactive specific binding member. As such, the term “specific binding molecule” encompasses antibody molecules (obtained from both polyclonal and monoclonal preparations), as well as, the following: hybrid (chimeric) antibody molecules (see, for example, Winter, et al., Nature 349:293-299 (1991), and U.S. Pat. No. 4,816,567); F(ab′)₂ and F(ab) fragments; Fv molecules (non-covalent heterodimers, see, for example, Inbar, et al., Proc. Natl. Acad. Sci. USA 69:2659-2662 (1972), and Ehrlich, et al., Biochem. 19:4091-4096 (1980)); single chain Fv molecules (sFv) (see, for example, Huston, et al., Proc. Natl. Acad. Sci. USA 85:5879-5883 (1988)); humanized antibody molecules (see, for example, Riechmann, et al., Nature 332:323-327 (1988), Verhoeyan, et al., Science 239:1534-1536 (1988), and UK Patent Publication NO. GB 2,276,169, published Sep. 21, 1994); and, any functional fragments obtained from such molecules, wherein such fragments retain immunological binding properties of the parent antibody molecule.

A “capture reagent,” as used herein, refers to an unlabeled specific binding member that is specific either for the analyte as in a sandwich assay, for the indicator reagent or analyte as in a competitive assay, or for an ancillary specific binding member, that itself is specific for the analyte, as in an indirect assay. The capture reagent can be directly or indirectly bound to a solid phase material before the performance of the assay or during the performance of the assay, thereby enabling the separation of immobilized complexes from the test sample.

The “indicator reagent” comprises a “signal-generating compound” (“label”) that is capable of generating and generates a measurable signal detectable by external means, conjugated (“attached”) to a specific binding member. In addition to being an antibody member of a specific binding pair, the indicator reagent also can be a member of any specific binding pair, including either hapten-anti-hapten systems such as biotin or anti-biotin, avidin or biotin, a carbohydrate or a lectin, a complementary nucleotide sequence, an effector or a receptor molecule, an enzyme cofactor and an enzyme, an enzyme inhibitor or an enzyme and the like. An immunoreactive specific binding member can be an antibody, an antigen, or an antibody/antigen complex that is capable of binding either to the polypeptide of interest as in a sandwich assay, to the capture reagent as in a competitive assay, or to the ancillary specific binding member as in an indirect assay. When describing probes and probe assays, the term “reporter molecule” may be used. A reporter molecule comprises a signal generating compound as described hereinabove conjugated to a specific binding member of a specific binding pair, such as carbazole or adamantane.

The various “signal-generating compounds” (labels) contemplated include chromagens, catalysts such as enzymes, luminescent compounds such as fluorescein and rhodamine, chemiluminescent compounds such as dioxetanes, acridiniums, phenanthridiniums and luminol, radioactive elements and direct visual labels. Examples of enzymes include alkaline phosphatase, horseradish peroxidase, beta-galactosidase and the like. The selection of a particular label is not critical, but it must be capable of producing a signal either by itself or in conjunction with one or more additional substances.

“Solid phases” (“solid supports”) are known to those in the art and include the walls of wells of a reaction tray, test tubes, polystyrene beads, magnetic or non-magnetic beads, nitrocellulose strips, membranes, microparticles such as latex particles, sheep (or other animal) red blood cells and Duracytes® (red blood cells “fixed” by pyruvic aldehyde and formaldehyde, available from Abbott Laboratories, Abbott Park, Ill.) and others. The “solid phase” is not critical and can be selected by one skilled in the art. Thus, latex particles, microparticles, magnetic or non-magnetic beads, membranes, plastic tubes, walls of microtiter wells, glass or silicon chips, sheep (or other suitable animal's) red blood cells and Duracytes® are all suitable examples. Suitable methods for immobilizing peptides on solid phases include ionic, hydrophobic, covalent interactions and the like. A “solid phase,” as used herein, refers to any material that is insoluble, or can be made insoluble by a subsequent reaction. The solid phase can be chosen for its intrinsic ability to attract and immobilize the capture reagent. Alternatively, the solid phase can retain an additional receptor that has the ability to attract and immobilize the capture reagent. The additional receptor can include a charged substance that is oppositely charged with respect to the capture reagent itself or to a charged substance conjugated to the capture reagent. As yet another alternative, the receptor molecule can be any specific binding member that is immobilized upon (attached to) the solid phase and which has the ability to immobilize the capture reagent through a specific binding reaction. The receptor molecule enables the indirect binding of the capture reagent to a solid phase material before the performance of the assay or during the performance of the assay. The solid phase thus can be a plastic, derivatized plastic, magnetic or non-magnetic metal, glass or silicon surface of a test tube, microtiter well, sheet, bead, microparticle, chip, sheep (or other suitable animal's) red blood cells, Duracytes® and other configurations known to those of ordinary skill in the art.

Reagents

The present invention provides reagents such as polynucleotide sequences derived from the AIPL1 gene or coding sequence or mutants thereof, polypeptides encoded thereby and antibodies specific for these polypeptides. The present invention also provides reagents such as oligonucleotide fragments derived from the disclosed polynucleotides and nucleic acid sequences complementary to these polynucleotides. The polynucleotides, polypeptides, or antibodies of the present invention may be used to provide information leading to the detecting, diagnosing, staging, monitoring, prognosticating, in vivo imaging, preventing or treating of, or determining the predisposition to, cancer and drug resistance. The sequences disclosed herein represent unique polynucleotides that can be used in assays or for producing a specific profile of gene transcription activity. Such assays are disclosed in European Patent Number 0373203B1 and International Publication No. WO 95/11995, which are hereby incorporated by reference.

Selected polynucleotides can be used in the methods described herein for the detection of normal or altered gene expression. Such methods may employ mutated or wild-type polynucleotides or oligonucleotides, fragments or derivatives thereof, or nucleic acid sequences complementary thereto.

The polynucleotides disclosed herein, their complementary sequences, or fragments of either, can be used in assays to detect, amplify or quantify genes, nucleic acids, cDNAs or mRNAs relating to LCA or other retinal diseases. They also can be used to identify an entire or partial coding region of a polypeptide. They further can be provided in individual containers in the form of a kit for assays, or provided as individual compositions. If provided in a kit for assays, other suitable reagents such as buffers, conjugates and the like may be included.

The polynucleotide may be in the form of RNA or DNA. Polynucleotides in the form of DNA, cDNA, genomic DNA, nucleic acid analogs and synthetic DNA are within the scope of the present invention. The DNA may be double-stranded or single-stranded, and if single stranded, may be the coding (sense) strand or non-coding (anti-sense) strand. The coding sequence that encodes the polypeptide may be identical to the coding sequence provided herein or may be a different coding sequence that coding sequence, as a result of the redundancy or degeneracy of the genetic code, encodes the same polypeptide as the DNA provided herein.

This polynucleotide may include only the coding sequence for the polypeptide, or the coding sequence for the polypeptide and an additional coding sequence such as a leader or secretory sequence or a proprotein sequence, or the coding sequence for the polypeptide (and optionally an additional coding sequence) and non-coding sequence, such as a non-coding sequence 5′ and/or 3′ of the coding sequence for the polypeptide.

In addition, the invention includes variant polynucleotides containing modifications such as polynucleotide deletions, substitutions or additions; and any polypeptide modification resulting from the variant polynucleotide sequence. A polynucleotide of the present invention also may have a coding sequence that is a naturally occurring allelic variant of the coding sequence provided herein.

In addition, the coding sequence for the polypeptide may be fused in the same reading frame to a polynucleotide sequence that aids in expression and secretion of a polypeptide from a host cell, for example, a leader sequence that functions as a secretory sequence for controlling transport of a polypeptide from the cell. The polypeptide having a leader sequence is a preprotein and may have the leader sequence cleaved by the host cell to form the polypeptide. The polynucleotides may also encode for a proprotein that is the protein plus additional 5′ amino acid residues. A protein having a prosequence is a proprotein and may, in some cases, be an inactive form of the protein. Once the prosequence is cleaved, an active protein remains. Thus, the polynucleotide of the present invention may encode for a protein, or for a protein having a prosequence, or for a protein having both a presequence (leader sequence) and a prosequence.

The polynucleotides of the present invention may also have the coding sequence fused in frame to a marker sequence that allows for purification of the polypeptide of the present invention. The marker sequence may be a hexa-histidine tag supplied by a pQE-9 vector to provide for purification of the polypeptide fused to the marker in the case of a bacterial host, or, for example, the marker sequence may be a hemagglutinin (HA) tag when a mammalian host, e.g. a COS-7 cell line, is used. The HA tag corresponds to an epitope derived from the influenza hemagglutinin protein. See, for example, I. Wilson et al., Cell 37:767 (1984).

When utilizing a hybridization-based detection system, a nucleic acid probe is chosen that is complementary to a target nucleic acid sequence, and then by selection of appropriate conditions the probe and the target sequence “selectively hybridize,” or bind, to each other to form a hybrid molecule. In one embodiment of the present invention, a nucleic acid molecule is capable of hybridizing selectively to a target sequence under moderately stringent hybridization conditions. In the context of the present invention, moderately stringent hybridization conditions allow detection of a target nucleic acid sequence of at least 14 nucleotides in length having at least approximately 70% sequence identity with the sequence of the selected nucleic acid probe. In another embodiment, such selective hybridization is performed under stringent hybridization conditions. Stringent hybridization conditions allow detection of target nucleic acid sequences of at least 14 nucleotides in length having a sequence identity of greater than 90% with the sequence of the selected nucleic acid probe. Hybridization conditions useful for probe/target hybridization where the probe and target have a specific degree of sequence identity, can be determined as is known in the art (see, for example, Nucleic Acid Hybridization: A Practical Approach, editors B. D. Hames and S. J. Higgins, (1985) Oxford; Washington, DC; IRL Press). Hybrid molecules can be formed, for example, on a solid support, in solution, and in tissue sections. The formation of hybrids can be monitored by inclusion of a reporter molecule, typically, in the probe. Such reporter molecules, or detectable elements include, but are not limited to, radioactive elements, fluorescent markers, and molecules to which an enzyme-conjugated ligand can bind.

With respect to stringency conditions for hybridization, it is well known in the art that numerous equivalent conditions can be employed to establish a particular stringency by varying, for example, the following factors: the length and nature of probe and target sequences, base composition of the various sequences, concentrations of salts and other hybridization solution components, the presence or absence of blocking agents in the hybridization solutions (e.g., formamide, dextran sulfate, and polyethylene glycol), hybridization reaction temperature and time parameters, as well as, varying wash conditions. The selection of a particular set of hybridization conditions is well within the skill of the routineer in the art (see, for example, Sambrook, et al., Molecular Cloning: A Laboratory Manual, Second Edition, (1989) Cold Spring Harbor, N.Y.).

“Stringent conditions” are defined as conditions that employ low ionic strength and high temerature for washing, for example, 0.015 M NaCl/0.015 M sodium citrate (SSC); 0.1% sodium lauryl sulfate (SDS) at 50 degrees C., or (2) employ a denaturing agent such as formamide during hyridization, e.g. 50% formamide with 0.1% bovine serum albumen/0.1% Ficoll/0.1%polyvinylpyrrolidone/50 mM sodium phosphate buffer at pH 6.5 with 750 mM NaCl, 75 mM sodium citrate at 42° C.

The present invention also provides an antibody produced by using a purified polypeptide of which at least a portion of the polypeptide is encoded by a polynucleotide selected from the polynucleotides provided herein. These antibodies may be used in the methods provided herein for the detection of antigen in test samples. The presence of antigen in the test samples is indicative of the presence of a retinal disease or condition. The antibody also may be used for therapeutic purposes, for example, in neutralizing the activity of polypeptide in conditions associated with altered or abnormal expression.

The present invention further relates to a polypeptide that has the deduced amino acid sequence as provided herein, as well as fragments, analogs and derivatives of such polypeptide. The polypeptide of the present invention may be a recombinant polypeptide, a natural purified polypeptide or a synthetic polypeptide. The fragment, derivative or analog of the polypeptide may be one in which one or more of the amino acid residues is substituted with a conserved or non-conserved amino acid residue (preferably a conserved amino acid residue) and such substituted amino acid residue may or may not be one encoded by the genetic code; or it may be one in which one or more of the amino acid residues includes a substituent group; or it may be one in which the polypeptide is fused with another compound, such as a compound to increase the half-life of the polypeptide (for example, polyethylene glycol); or it may be one in which the additional amino acids are fused to the polypeptide, such as a leader or secretory sequence or a sequence that is employed for purification of the polypeptide or a proprotein sequence. Such fragments, derivatives and analogs are within the scope of the present invention. The polypeptides and polynucleotides of the present invention are provided preferably in an isolated form and preferably purified.

Thus, a polypeptide of the present invention may have an amino acid sequence that is identical to that of the naturally occurring polypeptide or that is different by minor variations due to one or more amino acid substitutions. The variation may be a “conservative change” typically in the range of about 1 to 5 amino acids, wherein the substituted amino acid has similar structural or chemical properties, e.g., replacement of leucine with isoleucine or threonine with serine. In contrast, variations may include nonconservative changes, e.g., replacement of a glycine with a tryptophan. Similar minor variations may also include amino acid deletions or insertions, or both. Guidance in determining which and how many amino acid residues may be substituted, inserted or deleted without changing biological or immunological activity may be found using computer programs well known in the art, for example, DNASTAR software (DNASTAR Inc., Madison Wis.).

Probes constructed according to the polynucleotide sequences of the present invention can be used in various assay methods to provide various types of analysis. For example, such probes can be used in fluorescent in situ hybridization (FISH) technology to perform chromosomal analysis, and used to identify cancer-specific structural alterations in the chromosomes, such as deletions or translocations that are visible from chromosome spreads or detectable using PCR-generated and/or allele specific oligonucleotides probes, allele specific amplification or by direct sequencing. Probes also can be labeled with radioisotopes, directly- or indirectly-detectable haptens, or fluorescent molecules, and utilized for in situ hybridization studies to evaluate the mRNA expression of the gene comprising the polynucleotide in tissue specimens or cells.

This invention also provides teachings as to the production of the polynucleotides and polypeptides provided herein.

Probe Assays

The sequences provided herein may be used to produce probes that can be used in assays for the detection of nucleic acids in test samples. The probes may be designed from conserved nucleotide regions of the polynucleotides of interest or from non-conserved nucleotide regions of the polynucleotide of interest. The design of such probes for optimization in assays is within the skill of the routineer. Generally, nucleic acid probes are developed from non-conserved or unique regions when maximum specificity is desired, and nucleic acid probes are developed from conserved regions when assaying for nucleotide regions that are closely related to, for example, different members of a multi-gene family or in related species like mouse and man.

The polymerase chain reaction (PCR) is a technique for amplifying a desired nucleic acid sequence (target) contained in a nucleic acid or mixture thereof. In PCR, a pair of primers is employed in excess to hybridize to the complementary strands of the target nucleic acid. The primers are each extended by a polymerase using the target nucleic acid as a template. The extension products become target sequences themselves, following dissociation from the original target strand. New primers then are hybridized and extended by a polymerase, and the cycle is repeated to geometrically increase the number of target sequence molecules. PCR is disclosed in U.S. Pat. Nos. 4,683,195 and 4,683,202, which are incorporated herein by reference.

The Ligase Chain Reaction (LCR) is an alternate method for nucleic acid amplification. In LCR, probe pairs are used that include two primary (first and second) and two secondary (third and fourth) probes, all of which are employed in molar excess to target. The first probe hybridizes to a first segment of the target strand, and the second probe hybridizes to a second segment of the target strand, the first and second segments being contiguous so that the primary probes abut one another in 5′ phosphate-3′ hydroxyl relationship, and so that a ligase can covalently fuse or ligate the two probes into a fused product. In addition, a third (secondary) probe can hybridize to a portion of the first probe and a fourth (secondary) probe can hybridize to a portion of the second probe in a similar abutting fashion. Of course, if the target is initially double stranded, the secondary probes also will hybridize to the target complement in the first instance. Once the ligated strand of primary probes is separated from the target strand, it will hybridize with the third and fourth probes that can be ligated to form a complementary, secondary ligated product. It is important to realize that the ligated products are functionally equivalent to either the target or its complement. By repeated cycles of hybridization and ligation, amplification of the target sequence is achieved. This technique is described more completely in EP-A-320 308 to K. Backman published Jun. 16, 1989 and EP-A-439 182 to K. Backman et al., published Jul. 31, 1991, both of which are incorporated herein by reference.

For amplification of mRNAs, it is within the scope of the present invention to reverse transcribe mRNA into cDNA followed by polymerase chain reaction (RT-PCR); or, to use a single enzyme for both steps as described in U.S. Pat. No. 5,322,770, that is incorporated herein by reference; or reverse transcribe mRNA into cDNA followed by asymmetric gap ligase chain reaction (RT-AGLCR) as described by R. L. Marshall et al., PCR Methods and Applications 4:80-84 (1994), which also is incorporated herein by reference.

Other known amplification methods that can be utilized herein include but are not limited to the so-called V “NASBA” or “3SR” technique described by J. C. Guatelli et al., Proc. Natl. Acad. Sci. USA 87:1874-1878 (1990) and also described by J. Compton, Nature 350 (No. 6313):91-92 (1991); Q-beta amplification as described in published European Patent Application (EPA) No. 4544610; strand displacement amplification (as described in G. T. Walker et al., Clin. Chem. 42:9-13 [1996]) and European Patent Application No. 684315; and target mediated amplification, as described in International Publication No. WO 93/22461.

Detection of mutations to AIPL1 may be accomplished using any suitable detection method, including those detection methods that are currently well known in the art, as well as detection strategies that may evolve later. Examples of the foregoing presently known detection methods are hereby incorporated herein by reference. See, for example, Caskey et al., U.S. Pat. No. 5,582,989, Gelfand et al., U.S. Pat. No. 5,210,015. Examples of such detection methods include target amplification methods as well as signal amplification technologies. An example of presently known detection methods would include the nucleic acid amplification technologies referred to as PCR, LCR, NASBA, SDA, RCR and TMA. See, for example, Caskey et al., U.S. Pat. No. 5,582,989, Gelfand et al., U.S. Pat. No. 5,210,015. All of the foregoing are hereby incorporated by reference. Detection may also be accomplished using signal amplification such as that disclosed in Snitman et al., U.S. Pat. No. 5,273,882. While the amplification of target or signal is preferred at present, it is contemplated and within the scope of the present invention that ultrasensitive detection methods that do not require amplification can be utilized herein.

Detection, both amplified and non-amplified, may be performed using a variety of heterogeneous and homogeneous detection formats. Examples of heterogeneous detection formats are disclosed in Snitman et al., U.S. Pat. No. 5,273,882, Albarella et al., in EP-84114441.9, Urdea et al., U.S. Pat. No. 5,124,246, Ullman et al. U.S. Pat. No. 5,185,243 and Kourilskyet et al., U.S. Pat. No. 4,581,333. All of the foregoing are hereby incorporated by reference. Examples of homogeneous detection formats are disclosed in, Caskey et al., U.S. Pat. No. 5,582,989, Gelfand et al., U.S. Pat. No. 5,210,015, which are incorporated herein by reference. Also contemplated and within the scope of the present invention is the use of multiple probes in the hybridization assay, which use improves sensitivity and amplification of the BS325 signal. See, for example, Caskey et al., U.S. Pat. No. 5,582,989, Gelfand et al., U.S. Pat. No. 5,210,015, which are incorporated herein by reference.

In one embodiment, the present invention generally comprises the steps of contacting a test sample suspected of containing a target polynucleotide sequence with amplification reaction reagents comprising an amplification primer, and a detection probe that can hybridize with an internal region of the amplicon sequences. Probes and primers employed according to the method provided herein are labeled with capture and detection labels, wherein probes are labeled with one type of label and primers are labeled with another type of label. Additionally, the primers and probes are selected such that the probe sequence has a lower melt temperature than the primer sequences. The amplification reagents, detection reagents and test sample are placed under amplification conditions whereby, in the presence of target sequence, copies of the target sequence (an amplicon) are produced. In the usual case, the amplicon is double stranded because primers are provided to amplify a target sequence and its complementary strand. The double stranded amplicon then is thermally denatured to produce single stranded amplicon members. Upon formation of the single stranded amplicon members, the mixture is cooled to allow the formation of complexes between the probes and single stranded amplicon members.

As the single stranded amplicon sequences and probe sequences are cooled, the probe sequences preferentially bind the single stranded amplicon members. This finding is counterintuitive given that the probe sequences generally are selected to be shorter than the primer sequences and therefore have a lower melt temperature than the primers. Accordingly, the melt temperature of the amplicon produced by the primers should also have a higher melt temperature than the probes. Thus, as the mixture cools, the re-formation of the double stranded amplicon would be expected. As previously stated, however, this is not the case. The probes are found to preferentially bind the single stranded amplicon members. Moreover, this preference of probe/single stranded amplicon binding exists even when the primer sequences are added in excess of the probes.

After the probe/single stranded amplicon member hybrids are formed, they are detected. Standard heterogeneous assay formats are suitable for detecting the hybrids using the detection labels and capture labels present on the primers and probes. The hybrids can be bound to a solid phase reagent by virtue of the capture label and detected by virtue of the detection label. In cases where the detection label is directly detectable, the presence of the hybrids on the solid phase can be detected by causing the label to produce a detectable signal, if necessary, and detecting the signal. In cases where the label is not directly detectable, the captured hybrids can be contacted with a conjugate that generally comprises a binding member attached to a directly detectable label. The conjugate becomes bound to the complexes and the conjugate's presence on the complexes can be detected with the directly detectable label. Thus, the presence of the hybrids on the solid phase reagent can be determined. Those skilled in the art will recognize that wash steps maybe employed to wash away unhybridized amplicon or probe as well as unbound conjugate.

In one embodiment, the heterogeneous assays can be conveniently performed using a solid phase support that carries an array of nucleic acid molecules. Such arrays are useful for high-throughput and/or multiplexed assay formats. Various methods for forming such arrays from pre-formed nucleic acid molecules, or methods for generating the array using in situ synthesis techniques, are generally known in the art. (See, for example, Dattagupta, et al., EP Publication No. 0 234, 726A3; Southern, U.S. Pat. No. 5,700,637; Pirrung, et al., U.S. Pat. No. 5,143,854; PCT International Publication No. WO 92/10092; and, Fodor, et al., Science 251:767-777 (1991)).

Although the target sequence is described as single stranded, it also is contemplated to include the case where the target sequence is actually double stranded but is merely separated from its complement prior to hybridization with the amplification primer sequences. In the case where PCR is employed in this method, the ends of the target sequences are usually known. In cases where LCR or a modification thereof is employed in the preferred method, the entire target sequence is usually known. Typically, the target sequence is a nucleic acid sequence such as, for example, RNA or DNA.

The method provided herein can be used in well-known amplification reactions that include thermal cycle reaction mixtures, particularly in PCR and gap LCR (GLCR). Amplification reactions typically employ primers to repeatedly generate copies of a target nucleic acid sequence, which target sequence is usually a small region of a much larger nucleic acid sequence. Primers are themselves nucleic acid sequences that are complementary to regions of a target sequence. Under amplification conditions, these primers hybridize or bind to the complementary regions of the target sequence. Copies of the target sequence typically are generated by the process of primer extension and/or ligation that utilizes enzymes with polymerase or ligase activity, separately or in combination, to add nucleotides to the hybridized primers and/or ligate adjacent probe pairs. The nucleotides that are added to the primers or probes, as monomers or preformed oligomers, are also complementary to the target sequence. Once the primers or probes have been sufficiently extended and/or ligated, they are separated from the target sequence, for example, by heating the reaction mixture to a “melt temperature” which is one in which complementary nucleic acid strands dissociate. Thus, a sequence complementary to the target sequence is formed.

A new amplification cycle then can take place to further amplify the number of target sequences by separating any double stranded sequences, allowing primers or probes to hybridize to their respective targets, extending and/or ligating the hybridized primers or probes and re-separating. The complementary sequences that are generated by amplification cycles can serve as templates for primer extension or filling the gap of two probes to further amplify the number of target sequences. Typically, a reaction mixture is cycled between 20 and 100 times, more typically, a reaction mixture is cycled between 25 and 50 times. The numbers of cycles can be determined by the routineer. In this manner, multiple copies of the target sequence and its complementary sequence are produced. Thus, primers initiate amplification of the target sequence when it is present under amplification conditions.

Generally, two primers that are complementary to a portion of a target strand and its complement are employed in PCR. For LCR, four probes, two of which are complementary to a target sequence and two of which are similarly complementary to the target's complement, are generally employed. In addition to the primer sets and enzymes previously mentioned, a nucleic acid amplification reaction mixture may also comprise other reagents that are well known and include but are not limited to: enzyme cofactors such as manganese; magnesium; salts; nicotinamide adenine dinucleotide (NAD); and deoxynucleotide triphosphates (dNTPs) such as, for example, deoxyadenine triphosphate, deoxyguanine triphosphate, deoxycytosine triphosphate and deoxythymine triphosphate.

While the amplification primers initiate amplification of the target sequence, the detection (or hybridization) probe is not involved in amplification. Detection probes are generally nucleic acid sequences or uncharged nucleic acid analogs such as, for example, peptide nucleic acids that are disclosed in International Publication No. WO 92/20702; morpholino analogs that are described in U.S. Pat. Nos. 5,185,444, 5,034,506 and 5,142,047; and the like. Depending upon the type of label carried by the probe, the probe is employed to capture or detect the amplicon generated by the amplification reaction. The probe is not involved in amplification of the target sequence and therefore may have to be rendered “non-extendible” in that additional dNTPs cannot be added to the probe. In and of themselves, analogs usually are non-extendible and nucleic acid probes can be rendered non-extendible by modifying the 3′ end of the probe such that the hydroxyl group is no longer capable of participating in elongation. For example, the 3′ end of the probe can be functionalized with the capture or detection label to thereby consume or otherwise block the hydroxyl group. Alternatively, the 3′ hydroxyl group simply can be cleaved, replaced or modified. U.S. patent application Ser. No. 07/049,061 filed Apr. 19, 1993 and incorporated herein by reference describes modifications that can be used to render a probe non-extendible.

The ratio of primers to probes is not important. Thus, either the probes or primers can be added to the reaction mixture in excess whereby the concentration of one would be greater than the concentration of the other. Alternatively, primers and probes can be employed in equivalent concentrations. Preferably, however, the primers are added to the reaction mixture in excess of the probes. Thus, primer to probe ratios of, for example, 5:1 and 20:1 are preferred.

While the length of the primers and probes can vary, the probe sequences are selected such that they have a lower melt temperature than the primer sequences. Hence, the primer sequences are generally longer than the probe sequences. Typically, the primer sequences are in the range of between 20 and 50 nucleotides long, more typically in the range of between 20 and 30 nucleotides long. The typical probe is in the range of between 10 and 25 nucleotides long.

Various methods for synthesizing primers and probes are well known in the art. Similarly, methods for attaching labels to primers or probes are also well known in the art. For example, it is a matter of routine to synthesize desired nucleic acid primers or probes using conventional nucleotide phosphoramidite chemistry and instruments available from Applied Biosystems, Inc., (Foster City, Calif.), DuPont (Wilmington, Del.), or Milligen (Bedford Mass.). Many methods have been described for labeling oligonucleotides such as the primers or probes of the present invention. Enzo Biochemical (New York, N.Y.) and Clontech (Palo Alto, Calif.) both have described and commercialized probe-labeling techniques. For example, a primary amine can be attached to a 3′ oligo terminus using 3′-Amine-ON CPG™ (Clontech, Palo Alto, Calif.). Similarly, a primary amine can be attached to a 5′ oligo terminus using Aminomodifier II® (Clontech). The amines can be reacted to various haptens using conventional activation and linking chemistries. In addition, copending applications U.S. Ser. No. 625,566, filed Dec. 11, 1990 and Ser. No. 630,908, filed Dec. 20, 1990, which are each incorporated herein by reference, teach methods for labeling probes at their 5′ and 3′ termini, respectively. International Publication Nos WO 92/10505, published Jun. 25, 1992, and WO 92/11388, published Jul. 9, 1992, teach methods for labeling probes at their 5′ and 3′ ends, respectively. According to one known method for labeling an oligonucleotide, a label-phosphoramidite reagent is prepared and used to add the label to the oligonucleotide during its synthesis. See, for example, N. T. Thuong et al., Tet. Letters 29(46):5905-5908 (1988); or J. S. Cohen et al., published U.S. patent application Ser. No. 07/246,688 (NTIS ORDER No. PAT-APPL-7-246,688) (1989). Preferably, probes are labeled at their 3′ and 5′ ends.

A capture label is attached to the primers or probes and can be a specific binding member, which forms a binding pair with the solid phase reagent's specific binding member. It will be understood that the primer or probe itself may serve as the capture label. For example, in the case where a solid phase reagent's binding member is a nucleic acid sequence, it may be selected such that it binds a complementary portion of the primer or probe to thereby immobilize the primer or probe to the solid phase. In cases where the probe itself serves as the binding member, those skilled in the art will recognize that the probe will contain a sequence or “tail” that is not complementary to the single stranded amplicon members. In the case where the primer itself serves as the capture label, at least a portion of the primer will be free to hybridize with a nucleic acid on a solid phase because the probe is selected such that it is not fully complementary to the primer sequence.

Generally, probe/single stranded amplicon member complexes can be detected using techniques commonly employed to perform heterogeneous immunoassays. Preferably, in this embodiment, detection is performed according to the protocols used by the commercially available Abbott LCx® instrumentation (Abbott Laboratories, Abbott Park, Ill.).

The primers and probes disclosed herein are useful in typical PCR assays, wherein the test sample is contacted with a pair of primers, amplification is performed, the hybridization probe is added, and detection is performed.

Another method provided by the present invention comprises contacting a test sample with a plurality of polynucleotides, wherein at least one polynucleotide is a BS325 molecule as described herein, hybridizing the test sample with the plurality of polynucleotides and detecting hybridization complexes. Hybridization complexes are identified and quantitated to compile a profile that is indicative of retinal tissue disease, such as LCA. Expressed RNA sequences may further be detected by reverse transcription and amplification of the DNA product by procedures well known in the art, including polymerase chain reaction (PCR).

Drug Screening

The present invention also provides a method of screening a plurality of compounds for specific binding to the mutated polypeptide(s), or any fragment thereof, to identify at least one compound that specifically binds the mutated polypeptide. Such a method comprises the steps of providing at least one compound; combining the polypeptide with each compound under suitable conditions for a time sufficient to allow binding; and detecting the polypeptide binding to each compound.

The polypeptide or peptide fragment employed in such a test may either be free in solution, affixed to a solid support, borne on a cell surface or located intracellularly. One method of screening utilizes eukaryotic or prokaryotic host cells that are stably transfected with recombinant nucleic acids, which can express the polypeptide or peptide fragment. A drug, compound, or any other agent may be screened against such transfected cells in competitive binding assays. For example, the formation of complexes between a polypeptide and the agent being tested can be measured in either viable or fixed cells.

The present invention thus provides methods of screening for drugs, compounds, or any other agent, which can be used to treat resistant diseases associated with these mutations. These methods comprise contacting the agent with a polypeptide or fragment thereof and assaying for either the presence of a complex between the agent and the polypeptide, or for the presence of a complex between the polypeptide and the cell. In competitive binding assays, the polypeptide typically is labeled. After suitable incubation, free (or uncomplexed) polypeptide or fragment thereof is separated from that present in bound form, and the amount of free or uncomplexed label is used as a measure of the ability of the particular agent to bind to the polypeptide or to interfere with the polypeptide/cell complex.

The present invention also encompasses the use of competitive screening assays in which neutralizing antibodies capable of binding polypeptide specifically compete with a test agent for binding to the polypeptide or fragment thereof. In this manner, the antibodies can be used to detect the presence of any polypeptide in the test sample that shares one or more antigenic determinants with a mutated polypeptide as provided herein.

Another technique for screening provides high throughput screening for compounds having suitable binding affinity to at least one polypeptide disclosed herein. Briefly, large numbers of different small peptide test compounds are synthesized on a solid phase, such as plastic pins or some other surface. The peptide test compounds are reacted with polypeptide and washed. Polypeptide thus bound to the solid phase is detected by methods well known in the art. Purified polypeptide can also be coated directly onto plates for use in the screening techniques described herein. In addition, non-neutralizing antibodies can be used to capture the polypeptide and immobilize it on the solid support. See, for example, EP 84/03564, published on Sep. 13, 1984, that is incorporated herein by reference.

The goal of rational drug design is to produce structural analogs of biologically active polypeptides of interest or of the small molecules including agonists, antagonists, or inhibitors with which they interact. Such structural analogs can be used to design drugs that are more active or stable forms of the polypeptide or which enhance or interfere with the function of a polypeptide in vivo. J. Hodgson, Bio/Technology 9:19-21 (1991), incorporated herein by reference.

For example, in one approach, the three-dimensional structure of a polypeptide, or of a polypeptide-inhibitor complex, is determined by x-ray crystallography, by computer modeling or, most typically, by a combination of the two approaches. Both the shape and charges of the polypeptide must be ascertained to elucidate the structure and to determine active site(s) of the molecule. Less often, useful information regarding the structure of a polypeptide may be gained by modeling based on the structure of homologous proteins. In both cases, relevant structural information is used to design analogous polypeptide-like molecules or to identify efficient inhibitors

Useful examples of rational drug design may include molecules which have improved activity or stability as shown by S. Braxton et al., Biochemistry 31:7796-7801 (1992), or which act as inhibitors, agonists, or antagonists of native peptides as shown by S. B. P. Athauda et al., J Biochem. (Tokyo) 113 (6):742-746 (1993), incorporated herein by reference.

It also is possible to isolate a target-specific antibody selected by an assay as described hereinabove, and then to determine its crystal structure. In principle this approach yields a pharmacophore upon which subsequent drug design can be based. It further is possible to bypass protein crystallography altogether by generating anti-idiotypic antibodies (“anti-ids”) to a functional, pharmacologically active antibody. As a mirror image of a mirror image, the binding site of the anti-id is an analog of the original receptor. The anti-id then can be used to identify and isolate peptides from banks of chemically or biologically produced peptides. The isolated peptides then can act as the pharmacophore (that is, a prototype pharmaceutical drug).

A sufficient amount of a recombinant polypeptide of the present invention may be made available to perform analytical studies such as X-ray crystallography. In addition, knowledge of the polypeptide amino acid sequence that is derivable from the nucleic acid sequence provided herein will provide guidance to those employing computer modeling techniques in place of, or in addition to, x-ray crystallography.

The present invention also is directed to antagonists and inhibitors of the polypeptides of the present invention. The antagonists and inhibitors are those, which inhibit or eliminate the function of the polypeptide. Thus, for example, an antagonist may bind to a polypeptide of the present invention and inhibit or eliminate its function. The antagonist, for example, could be an antibody against the polypeptide, which eliminates the activity of a mutant polypeptide by binding a mutant polypeptide, or in some cases the antagonist may be an oligonucleotide. Examples of small molecule inhibitors include, but are not limited to, small peptides or peptide-like molecules.

The antagonists and inhibitors may be employed as a composition with a pharmaceutically acceptable carrier including, but not limited to, saline, buffered saline, dextrose, water, glycerol, ethanol and combinations thereof. Administration of polypeptide inhibitors is preferably systemic. The present invention also provides an antibody that inhibits the action of such a polypeptide.

Recombinant Technology

The present invention provides host cells and expression vectors comprising mutated polynucleotides of the present invention and methods for the production of the polypeptide(s) they encode. Such methods comprise culturing the host cells under conditions suitable for the expression of the mutant polynucleotide and recovering the mutant polypeptide from the cell culture.

The present invention also provides vectors that include mutant polynucleotides of the present invention, host cells that are genetically engineered with vectors of the present invention and the production of polypeptides of the present invention by recombinant techniques.

Host cells are genetically engineered (transfected, transduced or transformed) with the vectors of this invention, which may be cloning vectors or expression vectors. The vector may be in the form of a plasmid, a viral particle, a phage, etc. The engineered host cells can be cultured in conventional nutrient media modified as appropriate for activating promoters, selecting transfected cells, or amplifying AIPL1 gene(s). The culture conditions, such as temperature, pH and the like, are those previously used with the host cell selected for expression, and will be apparent to the ordinarily skilled artisan.

The polynucleotides of the present invention may be employed for producing a polypeptide by recombinant techniques. Thus, the polynucleotide sequence maybe included in any one of a variety of expression vehicles, in particular, vectors or plasmids for expressing a polypeptide. Such vectors include chromosomal, nonchromosomal and synthetic DNA sequences, e.g., derivatives of SV40; bacterial plasmids; phage DNA; yeast plasmids; vectors derived from combinations of plasmids and phage DNA, viral DNA such as vaccinia, adenovirus, fowl pox virus and pseudorabies. However, any other plasmid or vector may be used so long as it is replicable and viable in the host.

The appropriate DNA sequence may be inserted into the vector by a variety of procedures. In general, the DNA sequence is inserted into appropriate restriction endonuclease sites by procedures known in the art. Such procedures and others are deemed to be within the scope of those skilled in the art. The DNA sequence in the expression vector is operatively linked to an appropriate expression control sequence(s) (promoter) to direct mRNA synthesis. Representative examples of such promoters include, but are not limited to, the LTR or the SV40 promoter, the E. coli lac or trp, the phage lambda P sub L promoter and other promoters known to control expression of genes in prokaryotic or eukaryotic cells or their viruses. The expression vector also contains a ribosome binding site for translation initiation and a transcription terminator. The vector may also include appropriate sequences for amplifying expression. In addition, the expression vectors preferably contain a gene to provide a phenotypic trait for selection of transfected host cells such as dihydrofolate reductase or neomycin resistance for eukaryotic cell culture, or such as tetracycline or ampicillin resistance in E. coli.

The vector containing the appropriate DNA sequence as hereinabove described, as well as an appropriate promoter or control sequence, may be employed to transfect an appropriate host to permit the host to express the protein. As representative examples of appropriate hosts, there may be mentioned: bacterial cells, such as E. coli, Salmonella typhimurium; Streptomyces sp.; fungal cells, such as yeast; insect cells, such as Drosophila and Sf9; animal cells, such as CHO, COS or Bowes melanoma; plant cells, etc. The selection of an appropriate host is deemed to be within the scope of those skilled in the art from the teachings provided herein.

More particularly, the present invention also includes recombinant constructs comprising one or more of the sequences as broadly described above. The constructs comprise a vector, such as a plasmid or viral vector, into which a sequence of the invention has been inserted, in a forward or reverse orientation. In a preferred aspect of this embodiment, the construct further comprises regulatory sequences including, for example, a promoter, operably linked to the sequence. Large numbers of suitable vectors and promoters are known to those of skill in the art and are commercially available. The following vectors are provided by way of example. Bacterial: pINCY (Incyte Pharmaceuticals Inc., Palo Alto, Calif.), pSPORT1 (Life Technologies, Gaithersburg, Md.), pQE70, pQE60, pQE-9 (Qiagen) pBs, phagescript, psiX174, pBluescript SK, pBsKS, pNH8a, pNH16a, pNH18a, pNH46a (Stratagene); pTrc99A, pKK223-3, pKK233-3, pDR540, pRIT5 (Pharmacia); Eukaryotic: pWLneo, pSV2cat, pOG44, pXT1, pSG (Stratagene) pSVK3, pBPV, pMSG, pSVL (Pharmacia). However, any other plasmid or vector may be used as long as it is replicable and viable in the host.

Promoter regions can be selected from any desired gene using CAT (chloramphenicol transferase) vectors or other vectors with selectable markers. Two appropriate vectors are pKK232-8 and pCM7. Particular named bacterial promoters include lacI, lacZ, T3, SP6, T7, gpt, lambda P sub R, P sub L and trp. Eukaryotic promoters include cytomegalovirus (CMV) immediate early, herpes simplex virus (HSV) thymidine kinase, early and late SV40, LTRs from retroviruses and mouse metallothionein-I. Selection of the appropriate vector and promoter is well within the level of ordinary skill in the art.

In a further embodiment, the present invention provides host cells containing the above-described construct. The host cell can be a higher eukaryotic cell, such as a mammalian cell, or a lower eukaryotic cell, such as a yeast cell, or the host cell can be a prokaryotic cell, such as a bacterial cell. Introduction of the construct into the host cell can be effected by calcium phosphate transfection, DEAE-Dextran mediated transfection, or electroporation [L. Davis et al., Basic Methods in Molecular Biology, 2nd edition, Appleton and Lang, Paramount Publishing, East Norwalk, Conn. (1994)].

The constructs in host cells can be used in a conventional manner to produce the gene product encoded by the recombinant sequence. Alternatively, the polypeptides of the invention can be synthetically produced by conventional peptide synthesizers.

Recombinant proteins can be expressed in mammalian cells, yeast, bacteria, or other cells, under the control of appropriate promoters. Cell-free translation systems can also be employed to produce such proteins using RNAs derived from the DNA constructs of the present invention. Appropriate cloning and expression vectors for use with prokaryotic and eukaryotic hosts are described by Sambrook et al., Molecular Cloning: A Laboratory Manual, Second Edition, (Cold Spring Harbor, N.Y., 1989), which is hereby incorporated by reference.

Transcription of a DNA encoding the polypeptide(s) of the present invention by higher eukaryotes is increased by inserting an enhancer sequence into the vector. Enhancers are cis-acting elements of DNA, usually about from 10 to 300 bp, that act on a promoter to increase its transcription. Examples include the SV40 enhancer on the late side of the replication origin (bp 100 to 270), a cytomegalovirus early promoter enhancer, a polyoma enhancer on the late side of the replication origin and adenovirus enhancers.

Generally, recombinant expression vectors will include origins of replication and selectable markers permitting transfection of the host cell, e.g., the ampicillin resistance gene of E. coli and S. cerevisiae TRP1 gene, and a promoter derived from a highly expressed gene to direct transcription of a downstream structural sequence. Such promoters can be derived from operons encoding glycolytic enzymes such as 3-phosphoglycerate kinase (PGK), alpha factor, acid phosphatase, or heat shock proteins, among others. The heterologous structural sequence is assembled in appropriate phase with translation initiation and termination sequences, and preferably, a leader sequence capable of directing secretion of translated protein into the periplasmic space or extracellular medium. Optionally, the heterologous sequence can encode a fusion protein including an N-terminal identification peptide imparting desired characteristics, e.g., stabilization or simplified purification of expressed recombinant product.

Useful expression vectors for bacterial use are constructed by inserting a structural DNA sequence encoding a desired protein together with suitable translation initiation and termination signals in operable reading phase with a functional promoter. The vector will comprise one or more phenotypic selectable markers and an origin of replication to ensure maintenance of the vector and to, if desirable, provide amplification within the host. Suitable prokaryotic hosts for transfection include E. coli, Bacillus subtilis, Salmonella typhimurium and various species within the genera Pseudomonas, Streptomyces and Staphylococcus, although others may also be employed as a routine matter of choice.

Useful expression vectors for bacterial use comprise a selectable marker and bacterial origin of replication derived from plasmids comprising genetic elements of the well known cloning vector pBR322 (ATCC 37017). Other vectors include but are not limited to PKK223-3 (Pharmacia Fine Chemicals, Uppsala, Sweden) and GEM1 (Promega Biotec, Madison, Wis.). These pBR322 “backbone” sections are combined with an appropriate promoter and the structural sequence to be expressed.

Following transfection of a suitable host and growth of the host to an appropriate cell density, the selected promoter is derepressed by appropriate means (e.g., temperature shift or chemical induction), and cells are cultured for an additional period. Cells are typically harvested by centrifugation, disrupted by physical or chemical means, and the resulting crude extract retained for further purification. Microbial cells employed in expression of proteins can be disrupted by any convenient method including freeze-thaw cycling, sonication, mechanical disruption, or use of cell lysing agents. Such methods are well known to the ordinary artisan.

Various mammalian cell culture systems can also be employed to express recombinant protein. Examples of mammalian expression systems include the COS-7 lines of monkey kidney fibroblasts described by Gluzman, Cell 23:175 (1981), and other cell lines capable of expressing a compatible vector, such as the C127, HEK-293, 3T3, CHO, HeLa and BHK cell lines. Mammalian expression vectors will comprise an origin of replication, a suitable promoter and enhancer and also any necessary ribosome binding sites, polyadenylation sites, splice donor and acceptor sites, transcriptional termination sequences and 5′ flanking nontranscribed sequences. DNA sequences derived from the SV40 viral genome, for example, SV40 origin, early promoter, enhancer, splice, and polyadenylation sites may be used to provide the required nontranscribed genetic elements. Representative, useful vectors include pRc/CMV and pcDNA3 (available from Invitrogen, San Diego, Calf.).

Polypeptides are recovered and purified from recombinant cell cultures by known methods including affinity chromatography, ammonium sulfate or ethanol precipitation, acid extraction, anion or cation exchange chromatography, phosphocellulose chromatography, hydrophobic interaction chromatography, hydroxyapatite chromatography or lectin chromatography. It is preferred to have low concentrations (approximately 0.1-5 MM) of calcium ion present during purification [Price, et al., J. Biol. Chem. 244:917 (1969)]. Protein refolding steps can be used, as necessary, in completing configuration of the polypeptide. Finally, high performance liquid chromatography (HPLC) can be employed for final purification steps.

Thus, polypeptides of the present invention may be naturally purified products expressed from a high expressing cell line, or a product of chemical synthetic procedures, or produced by recombinant techniques from a prokaryotic or eukaryotic host (for example, by bacterial, yeast, higher plant, insect and mammalian cells in culture). Depending upon the host employed in a recombinant production procedure, the polypeptides of the present invention may be glycosylated with mammalian or other eukaryotic carbohydrates or may be non-glycosylated. The polypeptides of the invention may also include an initial methionine amino acid residue.

The starting plasmids can be constructed from available plasmids in accord with published, known procedures. In addition, equivalent plasmids to those described are known in the art and will be apparent to one of ordinary skill in the art.

The following is the general procedure for the isolation and analysis of cDNA clones. In a particular embodiment disclosed herein, mRNA is isolated from tissue and used to generate the cDNA library. Tissue is obtained from patients by surgical resection and is classified as tumor or non-tumor tissue by a pathologist.

Methods for DNA sequencing are well known in the art. Conventional enzymatic methods employ DNA polymerase, Klenow fragment, Sequenase (US Biochemical Corp, Cleveland, Ohio) or Taq polymerase to extend DNA chains from an oligonucleotide primer annealed to the DNA template of interest. Methods have been developed for the use of both single-stranded and double-stranded templates. The chain termination reaction products may be electrophoresed on urea/polyacrylamide gels and detected either by autoradiography (for radionucleotide labeled precursors) or by fluorescence (for fluorescent-labeled precursors). Recent improvements in mechanized reaction preparation, sequencing and analysis using the fluorescent detection method have permitted expansion in the number of sequences that can be determined per day using machines such as the Applied Biosystems 377 DNA Sequencers (Applied Biosystems, Foster City, Calif.).

The reading frame of the nucleotide sequence can be ascertained by several types of analyses. First, reading frames contained within the coding sequence can be analyzed for the presence of start codon ATG and stop codons TGA, TAA or TAG. Typically, one reading frame will continue throughout the major portion of a cDNA sequence while other reading frames tend to contain numerous stop codons. In such cases, reading frame determination is straightforward. In other more difficult cases, further analysis is required.

Algorithms have been created to analyze the occurrence of individual nucleotide bases at each putative codon triplet. See, for example J. W. Fickett, Nuc. Acids Res. 10:5303 (1982). Coding DNA for particular organisms (bacteria, plants and animals) tends to contain certain nucleotides within certain triplet periodicities, such as a significant preference for pyrimidines in the third codon position. These preferences have been incorporated into widely available software which can be used to determine coding potential (and frame) of a given stretch of DNA. The algorithm-derived information combined with start/stop codon information can be used to determine proper frame with a high degree of certainty. This, in turn, readily permits cloning of the sequence in the correct reading frame into appropriate expression vectors.

The nucleic acid sequences disclosed herein may be joined to a variety of other polynucleotide sequences and vectors of interest by means of well-established recombinant DNA techniques. See J. Sambrook et al., supra. Vectors of interest include cloning vectors, such as plasmids, cosmids, phage derivatives, phagemids, as well as sequencing, replication and expression vectors, and the like. In general, such vectors contain an origin of replication functional in at least one organism, convenient restriction endonuclease digestion sites and selectable markers appropriate for particular host cells. The vectors can be transferred by a variety of means known to those of skill in the art into suitable host cells that then produce the desired DNA, RNA or polypeptides.

Occasionally, sequencing or random reverse transcription errors will mask the presence of the appropriate open reading frame or regulatory element. In such cases, it is possible to determine the correct reading frame by attempting to express the polypeptide and determining the amino acid sequence by standard peptide mapping and sequencing techniques. See, F. M. Ausubel et al., Current Protocols in Molecular Biology, John Wiley & Sons, New York, N.Y. (1989). Additionally, the actual reading frame of a given nucleotide sequence may be determined by transfection of host cells with vectors containing all three potential reading frames. Only those cells with the nucleotide sequence in the correct reading frame will produce a peptide of the predicted length.

The nucleotide sequences provided herein have been prepared by current, state-of-the-art, automated methods and, as such, may contain unidentified nucleotides. These will not present a problem to those skilled in the art who wish to practice the invention. Several methods employing standard recombinant techniques, described in J. Sambrook (supra) or periodic updates thereof, may be used to complete the missing sequence information. The same techniques used for obtaining a full length sequence, as described herein, may be used to obtain nucleotide sequences.

Expression of a particular cDNA may be accomplished by subcloning the cDNA into an appropriate expression vector and transfecting this vector into an appropriate expression host. The cloning vector used for the generation of the retina cDNA library can be used for transcribing mRNA of a particular cDNA and contains a promoter for beta-galactosidase, an amino-terminal met and the subsequent seven amino acid residues of beta-galactosidase. Immediately following these eight residues is an engineered bacteriophage promoter useful for artificial priming and transcription, as well as a number of unique restriction sites, including EcoRI, for cloning. The vector can be transfected into an appropriate host strain of E. coli.

Induction of the isolated bacterial strain with isopropylthiogalactoside (IPTG) using standard methods will produce a fusion protein that contains the first seven residues of beta-galactosidase, about 15 residues of linker and the peptide encoded within the cDNA. Since cDNA clone inserts are generated by an essentially random process, there is one chance in three that the included cDNA will lie in the correct frame for proper translation. If the cDNA is not in the proper reading frame, the correct frame can be obtained by deletion or insertion of an appropriate number of bases by well known methods including in vitro mutagenesis, digestion with exonuclease III or mung bean nuclease, or oligonucleotide linker inclusion.

The cDNA can be shuttled into other vectors known to be useful for expression of protein in specific hosts. Oligonucleotide primers containing cloning sites and segments of DNA sufficient to hybridize to stretches at both ends of the target cDNA can be synthesized chemically by standard methods. These primers can then be used to amplify the desired gene segments by PCR. The resulting new gene segments can be digested with appropriate restriction enzymes under standard conditions and isolated by gel electrophoresis. Alternately, similar gene segments can be produced by digestion of the cDNA with appropriate restriction enzymes and filling in the missing gene segments with chemically synthesized oligonucleotides. Segments of the coding sequence from more than one gene can be ligated together and cloned in appropriate vectors to optimize expression of recombinant sequence.

Suitable expression hosts for such chimeric molecules include, but are not limited to, mammalian cells, such as Chinese Hamster Ovary (CHO) and human embryonic kidney (HEK) 293 cells, insect cells, such as Sf9 cells, yeast cells, such as Saccharomyces cerevisiae and bacteria, such as E. col. For each of these cell systems, a useful expression vector may also include an origin of replication to allow propagation in bacteria and a selectable marker such as the beta-lactamase antibiotic resistance gene to allow selection in bacteria. In addition, the vectors may include a second selectable marker, such as the neomycin phosphotransferase gene, to allow selection in transfected eukaryotic host cells. Vectors for use in eukaryotic expression hosts may require the addition of 3′ poly A tail if the sequence of interest lacks poly A.

Additionally, the vector may contain promoters or enhancers that increase gene expression. Such promoters are host specific and include, but are not limited to, MMTV, SV40, or metallothionine promoters for CHO cells; trp, lac, tac or T7 promoters for bacterial hosts; or alpha factor, alcohol oxidase or PGH promoters for yeast. Adenoviral vectors with or without transcription enhancers, such as the Rous sarcoma virus (RSV) enhancer, may be used to drive protein expression in mammalian cell lines. Once homogeneous cultures of recombinant cells are obtained, large quantities of recombinantly produced protein can be recovered from the conditioned medium and analyzed using chromatographic methods well known in the art. An alternative method for the production of large amounts of secreted protein involves the transfection of mammalian embryos and the recovery of the recombinant protein from milk produced by transgenic cows, goats, sheep, etc. Polypeptides and closely related molecules may be expressed recombinantly in such a way as to facilitate protein purification. One approach involves expression of a chimeric protein which includes one or more additional polypeptide domains not naturally present on human polypeptides. Such purification-facilitating domains include, but are not limited to, metal-chelating peptides such as histidine-tryptophan domains that allow purification on immobilized metals, protein A domains that allow purification on immobilized immunoglobulin and the domain utilized in the FLAGS extension/affinity purification system (Immunex Corp, Seattle, Wash.). The inclusion of a cleavable linker sequence such as Factor XA or enterokinase from Invitrogen (San Diego, Calif.) between the polypeptide sequence and the purification domain may be useful for recovering the polypeptide.

It is contemplated and within the scope of the present invention that the solid phase also can comprise any suitable porous material with sufficient porosity to allow access by detection antibodies and a suitable surface affinity to bind antigens. Microporous structures generally are preferred, but materials with a gel structure in the hydrated state may be used as well. Such useful solid supports include, but are not limited to, nitrocellulose and nylon. It is contemplated that such porous solid supports described herein preferably are in the form of sheets of thickness from about 0.01 to 0.5 mm, preferably about 0.1 mm. The pore size may vary within wide limits and preferably is from about 0.025 to 15 microns, especially from about 0.15 to 15 microns. The surface of such supports may be activated by chemical processes that cause covalent linkage of the antigen or antibody to the support. The irreversible binding of the antigen or antibody is obtained, however, in general, by adsorption on the porous material by poorly understood hydrophobic forces. Other suitable solid supports are known in the art.

In addition to the above methods, the expression vectors of the present invention can also be introduced using Liposome-Mediated Transfection. The therapeutic potential for liposome-mediated gene transfer into retinal tissue to ameliorate disease symptoms has been successfully demonstrated for other genes such as NGF and BDNF as disclosed in U.S. Pat. No. 6,096,716, incorporated herein by reference. Liposome-mediated transfection is a non-toxic systemic injection of cDNA:cationic liposome complexes into animals. Such methods have proven to be superior to those methods of the prior art in the transfection of post-mitotic cells. Moreover, the present invention has demonstrated that liposome-mediated gene transfer can be used to effectively incorporate large gene inserts. Specific tissues and cell types may be targeted in vivo by the use of selected promoter-enhancer elements that are tissue and cell type specific, administration of the plasmid regionally into selected tissue compartments and coupling a targeting ligand to the liposomal surface.

The present invention provides for a liposomal-mediated system for transfecting cDNA of AIPL1 into a retinal cells of a retinal site, and represents the first successful use of cationic liposomes as efficient and clinically relevant vectors for the transfer of genes into cells of the central nervous system. Efficient transfection of genes may result in therapeutic levels of expression of AIPL1 and other proteins which could be useful in the treatment of a variety of retinal pathologies including LCA and other retinal diseases.

Technology for introduction of DNA into cells is well-known to those of skill in the art. Four general methods for delivering a gene into cells have been described: (1) chemical methods; (2) physical methods such as microinjection, electroporation and the gene gun; (3) viral vectors; and (4) receptor-mediated mechanisms.

Chemical and physical methods of DNA transfer are relatively inefficient processes and are not applicable to studies in which gene transfer needs to occur in a relatively high percentage of cells. Therefore, much effort has focused on developing viral vectors for gene transfer and on developing new compounds, such as liposomes, that would allow DNA transfer at relatively high efficiency. Important clinical disadvantages of viral vectors include the possibility of replication-competent virus production, immunological reactions and toxicity. Liposomes have also been used successfully with a number of cell types that are normally resistant to transfection by other procedures including T cell suspensions, primary hepatocyte cultures and PC 12 cells. In addition, liposomes are free of the DNA length constraints that are typical of viral-based delivery systems. Liposomes have been used effectively to introduce genes, drugs, radiotherapeutic agents, enzymes, viruses, transcription factors and allosteric effectors into a variety of cultured cell lines and animals. In addition, several successful clinical trails examining the effectiveness of liposome-mediated drug delivery have been completed. Furthermore, several studies suggest that the use of liposomes is not associated with autoimmune responses, toxicity or gonadal localization after systemic delivery.

Introduction of the liposome-cDNA transfection complex may be by injection, and may be systemic injections into peripheral arteries or veins, including the carotid or jugular vessels. Injection may also be directly into the eye or retina or associated tissue, either by intraventricular administration, or directly into the retinal tissue itself. Such injection may be facilitated by the use of mini-osmotic pumps for long-duration infusion, or an intraparenchymal injection apparatus with ventricular cannuli or other intraparenchymal devices. In other embodiments, it may be desirable to introduce the liposome-cDNA complex directly into the retina or surrounding tissue.

AIPL1 Mapping and Identification

During an effort to identify and map genes expressed exclusively in the retina and pineal gland as candidates for inherited retinal degeneration, AIPL1 was mapped to 17p13 (Sohocki M M, Malone K A, Sullivan L S, Daiger S P. Localization of retina/pineal-expressed sequences (ESTs): identification of novel candidate genes for inherited retinal disorders. Genomics 58:29-33, 1999), near the candidate region of several inherited retinal degenerations for which the cause remained unknown, including RP13 (Greenberg J, Goliath R, Beighton P, Ramesar R. A new locus for autosomal dominant retinitis pigmentosa on the short arm of chromosome 17. Hum Mol Genet 3:915-918, 1994), CORD5 (Balciuniene J, Johnsson K, Sandgren O, Wachtmeister L, Holmgren G, Forsman K. A gene for autosomal dominant progressive cone dystrophy (CORD5) maps to chromosome 17p 12-p 13. Genomics 30:281-286, 1995), and LCA4 (Hameed A, Khaliq S, Ismail M, Anwar K, Ebenezer N D, Jordan T, Mehdi S Q, Payne A M, Bhattacharya S S. A novel locus for Leber congenital amaurosis with anterior keratoconus mapping to 17p13. Invest Ophthalmo Vis Sci 41:629-633, 2000). AIPL1 includes six exons as shown in Table 1 encoding a 384 amino acid protein which belongs to the FK506-binding protein (FKBP) family.

TABLE 1
Intron/exon organization of AIPL1

| | Exon | Starting | | |
|---|---|---|---|---|
| Exon/ | Length | position | | |
| Intron | (bp) | in cDNA^{a} | Acceptor splice site^{b} | Donor splice site^{b} |
| 1 | 96 | 1 | | CGGATCCCGAgtgagtggggccctccggagcaga |
| 2 | 180 | 97 | cagagtgcaccgtctcggtgactagGTGATCTTTC | CSACACCATCgtaagtaggccctgcgcgcctgtct |
| 3 | 189 | 277 | gccatccatccgtttatccccacagCACACGGGGG | GCTGCTGCAGgtggggctggggttggcagggctgg |
| 4 | 177 | 466 | cactgacctgcagctctggggccagGTTGATGCCC | GCAGACCAAGgtcagaggccgctggccacggggtg |
| 5 | 142 | 643 | catggctgaccttctccctgggcagGAGAAGCCRT | CACCACCCAGgtgcgcggggctgcaggggcggaca |
| 6 | 754/1563^{c} | 785 | gctggatgctccctgctccccacagGCATCGTGAA | |

^{a}Numbering based on cDNA sequence, with position 1 as the first base of the ATG start codon.
^{b}The exonic and intronic sequences are represented in upper and lowercase, respectively.
^{c}Lengths differ by 709 bp in the 3′ untranslated region due to alternate polyadenylation signal usage.

AIPL1 contains three tetratricopeptide (TPR) motifs, 34 amino acid motifs found in proteins that mediate a variety of functions, including nuclear transport or protein chaperone activity (Ma Q, Whitlock J P. A novel cytoplasmic protein that interacts with the Ah receptor, contains tetratricopeptide repeat motifs, and augments the transcriptional response to 2,3,7,8-tetrachlorodibenzo-p-dioxin. J Biol Chem 272:8878-8884, 1997). Therefore, AIPL1 may perform a similar function; however, the inventors believe that mutant AIPL1 proteins may be involved in retinal diseases including LCA and the wild-type protein may be an effective agent in ameliorating symptoms of LCA and other retinal diseases. AIPL1 also includes a 56 amino acid proline-rich “hinge” region near the carboxyl-terminus of the protein that appears to be present only in primate aipl1.

In a first study, the inventors demonstrated that AIPL1 mutations cause the retinal disorder in the original LCA4 family, as well as in three of fourteen families whose LCA was not tested by linkage. In a second study, the inventors screened AIPL1 in a large cohort of patients with a wide range of clinical diagnoses as shown in Table 2. The inventors have identified AIPL1 mutations as the cause of LCA in 11 families, as well as identified an apparently dominant 12 base pair (bp) deletion within the “hinge” region of the protein which causes retinal dystrophy in two families.

TABLE 2
Clinical Diagnoses of Individuals Screened for AIPL1 mutations
in this study (Total = 512)

| | No. of Unrelated |
|---|---|
| Clinical Diagnosis | Probands Tested |
| Retinitis pigmentosa, autosomal dominant | 186 |
| Retinitis pigmentosa, autosomal recessive | 11 |
| Retinitis pigmentosa, isolated | 70 |
| Cone-rod dystrophy, autosomal dominant | 15 |
| Cone-rod dystrophy, autosomal recessive | 2 |
| Cone-rod dystrophy, isolated | 38 |
| Leber congenital amaurosis, recessive or isolated | 188 |
| Usher syndrome, autosomal recessive | 2 |

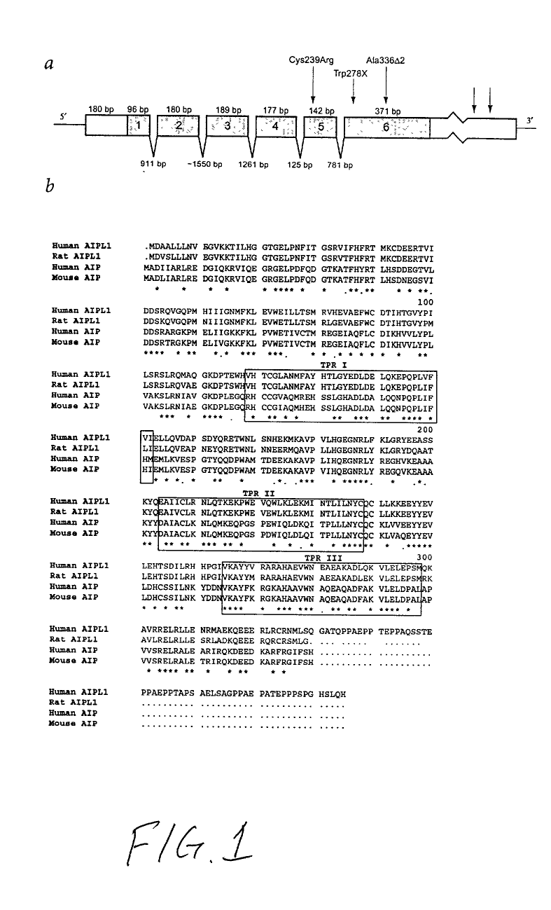

cDNA sequencing of the two clusters indicated that the ESTs represent transcripts of one gene. THC90422 transcripts bypass the THC220430 polyadenylation signal, resulting in a 709 bp longer 3′ untranslated region (UTR). The 180 bp 5′ UTR and coding sequence encoded by the six-exon gene are identical in the 1538 bp and 2247 bp transcripts as shown in FIG. 1a. AIPL1 comprises six exons, with alternate polyadenylation sites in the 3′ untranslated region, shown by arrows. Cys239Arg denotes the location of the TGC→CGC missense mutation in exon 5 of the RFS128 family. Trp278X denotes the location of the TGG→TGA nonsense mutation in exon 6 of the KC, MD, RFS127 and RFS121 families. Ala336Δ2 denotes the location of the 2 bp deletion in exon 6 of RFS121. Benign coding sequence substitutions identified were Phe37Phe (TTT/TTC; 0.98/0.02 frequency), Cys89Cys (TGC/TGT; 0.99/0.01), Asp90His (GAC/CAC; 0.84/0.16), Leu100Leu (CTG/CTA; 0.57/0.43) and Pro217Pro (CCG/CCA; 0.61/0.39).

The inventors have named the gene “human aryl hydrocarbon receptor-interacting protein-like 1” (AIPL1) due to its extensive similarity (49% identity, 69% positive) to human aryl hydrocarbon receptor-interacting protein (AIP), a member of the FK506-binding protein (FKBP) family (Ma, Q. & Whitlock Jr., J. P. A novel cytoplasmic protein that interacts with the Ah receptor, contains tetratricopeptide repeat motifs, and augments the transcriptional response to 2,3,7,8-tetrachlorodibenzo-p-dioxin. J. Biol. Chem. 272, 8878-8884 (1997)) as shown in FIG. 1b. The protein sequence of AIPL1 alignment demonstrates the high level of sequence conservation between rat and human AIPL1, and mouse and human AIP. Identical residues in the four sequences are noted with an asterisk; identical residues in three of the sequences are indicated with a period.

The predicted protein comprises 384 amino acids, with a 43,865 Dalton molecular mass, and a 5.57 pI. The protein sequence includes three tetratricopeptide repeats (TPR), a 34 amino acid motif found in proteins with nuclear transport or protein chaperone activity (Ma, Q. & Whitlock Jr., J. P. A novel cytoplasmic protein that interacts with the Ah receptor, contains tetratricopeptide repeat motifs, and augments the transcriptional response to 2,3,7,8-tetrachlorodibenzo-p-dioxin. J. Biol. Chem. 272, 8878-8884 (1997)).

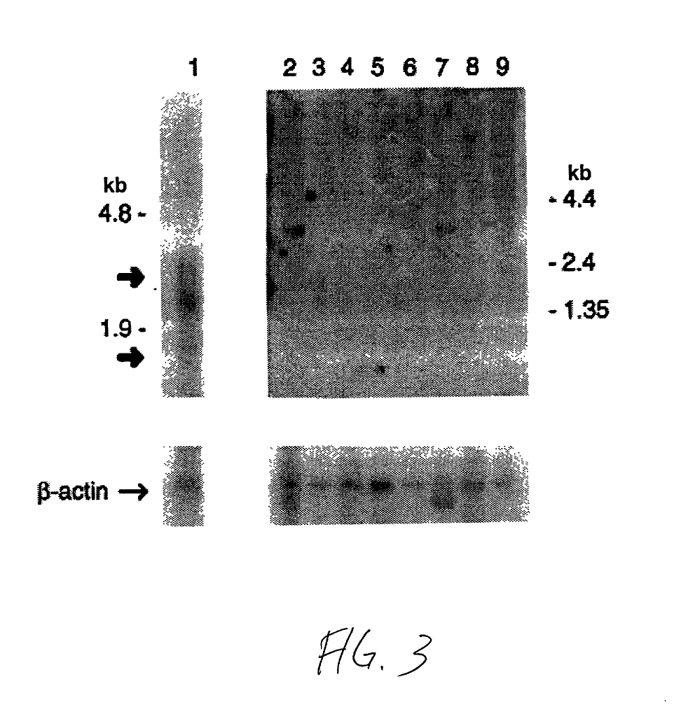

Northern hybridization identified mRNA molecules of the predicted sizes in total retinal RNA. The probe also cross-hybridized to 18s rRNA as shown in FIG. 3 in the retina. The Northern blots from adult tissues were incubated with an AIPL1 probe as shown in FIG. 3. Total retinal RNA blot, exposed 4 hours at 70° C. (upper left) and polyA⁺ RNA multi-tissue Northern (MTN), exposed 72 hours at 70° C. (upper right). No signal was observed in MTN at 4, 24, or 48-hour exposure. Lane 1, adult retina; lane 2, heart; lane 3, whole brain; lane 4, placenta; lane 5, lung; lane 6, liver; lane 7, skeletal muscle; lane 8, kidney; lane 9, pancreas. Both blots were incubated with a β-actin probe as a control (lower panel). Solid arrows indicate mRNA molecules of the predicted sizes, 1538 and 2247 bp, in retina. A weaker signal was detected in skeletal muscle and heart on a polyA⁺ RNA multi-tissue Northern after very long exposure. It is likely that this signal represents cross-hybridization, as the transcripts differ in size from the retinal mRNAs, and are faint. The Northern did not indicate AIPL1 expression in brain; however, only cerebral tissue was included in the blot.

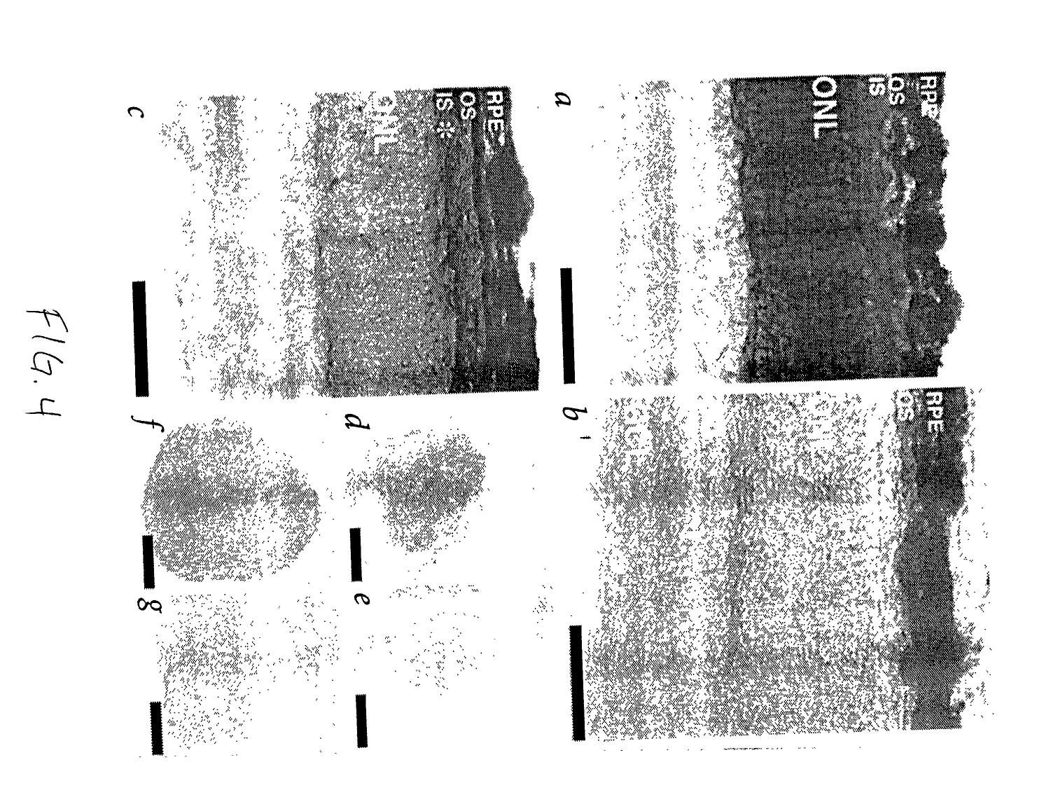

In situ hybridization indicated expression in rat and mouse pineal, a high level of expression in adult mouse photoreceptors as shown in FIG. 4, and no expression in cornea (data not shown). FIG. 4a depicts digoxygenin in situ hybridization of Aipl1 in adult mouse retina, with expression throughout the outer nuclear layer and photoreceptor inner segments. Color reaction time is 4 days. FIG. 4b depicts sense control of “a” with same reaction time. A slight background signal is observed across photoreceptor outer segments. FIG. 4c depicts short (16 hour) color reaction of Aipl1 in adult mouse retina, showing a high level of mRNA in photoreceptor inner segments. FIG. 4d depicts expression of Aipl1 in adult mouse pineal. Color reaction time is 4 days. FIG. 4e depicts sense control of “d”, with same reaction time. FIG. 4f depicts expression of Aipl1 mRNA in P14 rat pineal. Color reaction time is 4 days. FIG. 4g depicts sense control of “f ”, with same reaction time. Scale bar for a-c is 30 μm, for d and e is 50 μm, and f and g is 70 μm. RPE-retinal pigment epithelium, OS-outer photoreceptor segment, IS-inner photoreceptor segment, ONL-outer nuclear layer, INL-inner nuclear layer, GCL-ganglion cell layer. Immunolocalization of the AIPL1 protein has not been performed; therefore, site of AIPL1 protein localization is currently unknown.

Sequencing of the rat Aipl1 cDNA revealed extensive amino acid sequence conservation (87% identity and 96% similarity) between rat and human AIPL1. Interestingly, rat Aipl1, mouse Aip, and human AIP lack a 56 amino acid carboxyl-terminal extension present in AIPL1 as shown in FIG. 1b; this extension includes a “hinge” motif of high flexibility, with multiple O-glycosylation sites, and a casein kinase II (CK2) phosphorylation site, which is thought to be involved in protein complex regulation, as is the CK2 site within the hinge of another FKBP family member, FKBP52 (Miyata, Y. et al. Phosphorylation of the immunosuppressant FK506-binding protein FKBP52 by casein kinase II: regulation of HSP90-binding activity of FKBP52. Proc. Natl. Acad. Sci. 94, 14500-14505 (1997)). The hinge appears to be conserved in primates, as it is also present in the squirrel monkey (Saimiri sciureus; data not shown).

Single-stranded conformational analysis (SSCA) identified three benign nucleotide substitutions within the AIPL1 exon 3 amplimer: G/A at—14, G/A at—10 bp, and G/A at codon 100 (Leu100Leu, CTG/CTA). Four haplotypes were identified for the combined polymorphisms; the most common, GCG and GAA, have frequencies of 55% and 41%, respectively.

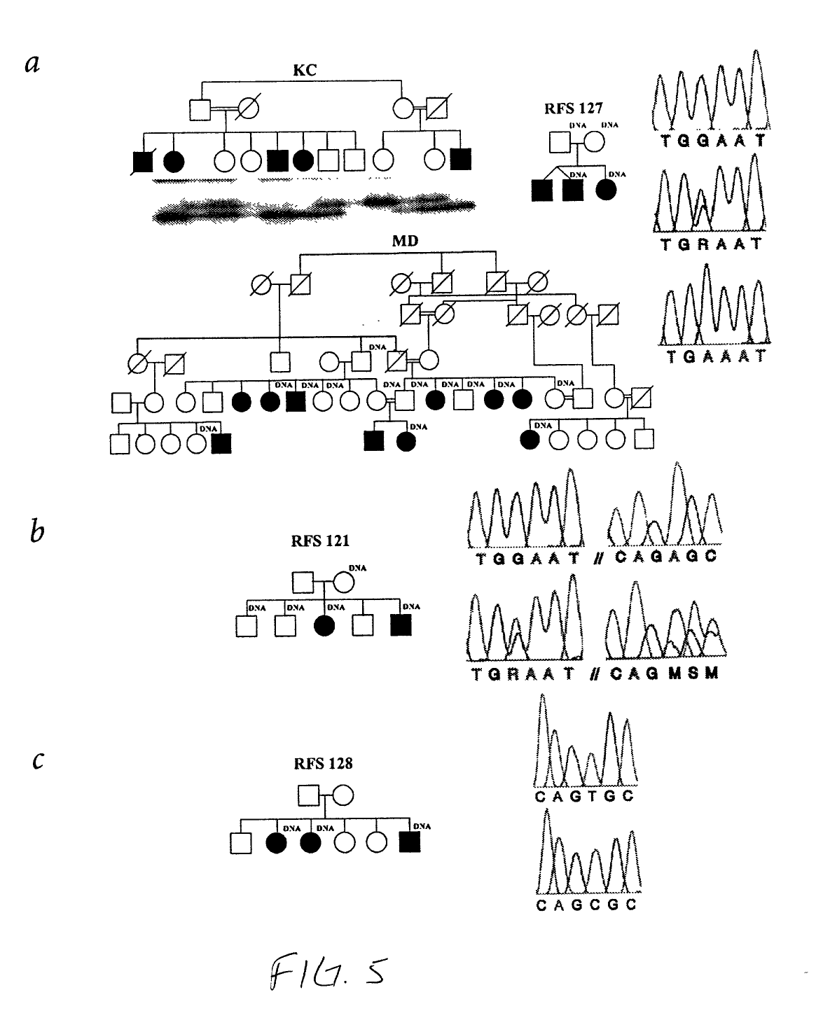

Referring now to FIG. 5, pedigrees and mutation screen of AIPL1 in families is shown. FIG. 5a depicts the Trp278X mutation is homozygous in three families: KC, MD and RFS127. SSCA of all living individuals of the KC pedigree demonstrate segregation of the mutant allele. Top electropherogram: an unaffected control (TGG/TGG). Middle: heterozygous G/A mutation at codon 278. Bottom: DNA sequence of a homozygous, affected member of MD (TGA/TGA).

FIG. 5b depicts the RFS121 affected individuals are compound heterozygotes for the Trp278X and Ala336Δ2 bp mutations. Top electropherogram: unaffected control, bottom: heterozygous G/A mutation at codon 278 (left) and heterozygous 2 bp deletion beginning in codon 336 (right) in an affected individual of RFS121.

FIG. 5c depicts the Cys239Arg mutation found in family RFS128. Top electopherogram: unaffected control (TGC/TGC), bottom: DNA sequence of a homozygous, affected individual (CGC/CGC).

Sequencing of AIPL1 from the DNA of one affected individual of the original LCA4 family as shown in FIG. 5a, revealed a homozygous nonsense mutation (Trp278X, TGG→TGA). This allele, if expressed, encodes a protein 107 amino acids shorter than wild-type AIPL1. The truncated protein includes only 20 of the 34 amino acids of the third TPR motif, a region conserved between human, rat and mouse AIPL1, and AIP. SSCA in other family members confirmed that all affected family members are homozygous for this mutation as shown in FIG. 5a, and that 100 ethnically-matched controls did not carry this mutation.

AIPL1 was next analyzed in another Pakistani family, MD as shown in FIG. 5a, whose LCA had been mapped to 17p13.1, with GUCY2D excluded by mutational analysis. Sequencing of AIPL1 indicated that affected individuals of this family are homozygous for the Trp278X mutation as shown in FIG. 5a. The MD and KC families differ in haplotype (GCG and GAA, respectively) of the AIPL1 exon 3 polymorphisms, as well as for microsatellite markers tightly linked to AIPL1. These results are though to suggest that the Trp278X mutations causing the LCA in these two families are not derived from a recent, common ancestor.

Assay of AIPL1 in fourteen Caucasian families with LCA that had not been tested previously for linkage to 17p identified apparent disease-causing mutations in three additional families, as follows.

Family RFS121

Direct sequencing of AIPL1 in the two affected RFS121 individuals indicated two mutations, a 2 bp deletion in codon 336 (Ala336Δ2 bp; see FIG. 5b) and the Trp278X mutation. The deletion results in a frame shift and a termination delayed by 47 codons. The termination signal used in the deletion transcript is upstream of the first AIPL1 polyadenylation signal; therefore, the alternate transcripts from this allele are not predicted to encode alternate proteins. Allele-specific PCR in one affected individual confirmed that the 2 bp deletion and Trp278X mutations are on opposite chromosomes. Therefore, the affected individuals in RFS121 are compound heterozygotes, having received the Trp278X mutation from one parent and the Ala336Δ2 mutation from the other. No unaffected RFS121 family members inherited both mutations. The Ala336Δ2 bp mutation was not observed in 55 unrelated Caucasian control individuals.

Family RFS127

AIPL1 sequencing from two affected RFS127 individuals as shown in FIG. 5a indicated homozygous Trp278X mutations—the same mutation identified in KC and MD. Haplotype analysis of tightly linked microsatellite markers, and of the AIPL1 exon 3 polymorphisms suggest that the mutations in the RFS 127 and MD families are likely to have descended from a common ancestor. However, there is no indication of Pakistani origin for members of this family.

Family RFS128

The three affected individuals of RFS128 as shown in FIG. 5c are homozygous for a T→C nucleotide substitution predicted to encode a Cys239Arg substitution. This cysteine is conserved in human and rat AIPL1, and in AIP as shown in FIG. 1. This mutation was not identified in over 55 ethnically-matched control individuals. Affected members of this family are homozygous for microsatellite markers D17S796 and D17S1881, tightly linked, flanking markers of AIPL1. In contrast, affected family members are heterozygous for microsatellite markers D17S960 and D17S1353, which flank GUCY2D.

These findings indicate that the inventors have identified a novel gene that causes LCA4, having detected homozygous AIPL1 mutations in three families in which GUCY2D was excluded as the cause of the disease by linkage and/or mutation screening: KC, MD and RFS128. AIPL1 is the fourth gene to be associated with LCA. Mutations in AIPL1 may be a common cause of LCA, as an AIPL1 mutation was identified as the apparent cause of the retinal disease in three of fourteen (21±8%, 90% C.I.) unmapped LCA families. The inventors believe that AIPL1 should be assayed in LCA families whose disease locus maps to 17p13, but with no apparent disease-causing mutations in GUCY2D, as in 7 of the 15 original LCA1 families (Marlhens, F. et al. Mutations in RPE65 cause Leber's congenital amaurosis. Nature Genet. 17, 139-141(1997)). Thus, screening for these mutation should be an effective method for detecting LCA or other retinal diseases early and to help advise patients at high risk to pass the propensity for LCA or other retinal diseases to their offspring.

Due to the proximity of AIPL1 and GUCY2D on 17p13, linkage mapping may not distinguish between the genes. Further, it is possible that LCA patients who are identical by descent (IBD) at one locus are also IBD at the other. Therefore, both AIPL1 and GUCY2D should be screened for mutations in families whose LCA locus maps to 17p13 or in families with affected individuals who are homozygous for mutations in either gene, unless linkage excludes one of the genes. Thus, the present invention also relates to a method for screen a patient or a patient population using probes designed to identify AIPL1 and GUCY2D mutation. Of the five families reported here, GUCY2D was excluded by linkage testing and/or mutation screening in three; the fourth is a compound heterozygote; and the fifth is homozygous for a disease-causing mutation confirmed in other families.

The similarity of AIPL1 to AIP and the presence of three TPR motifs suggest that it may be involved in retinal protein folding and/or trafficking. Its role in the pineal gland is also uncertain. The pineal gland contributes to resetting circadian rhythm by diurnal release of melatonin. Additionally, children with destructive pinealomas often display precocious puberty, suggesting a role in long-term periodicity (Endocrine role of the pineal gland. in Endocrinology (ed. Hadley, M. E.) 458-476 (New York, N.Y., 1996)). Because LCA patients with AIPL1 mutations have grossly abnormal photoreceptors at an early age, the pineal gland also may be affected. Careful clinical characterization of LCA4 patients may reveal pineal-associated abnormalities. Therefore, identifying the exact role of AIPL1 in photoreceptors and the pineal gland will improve our understanding of disease pathology in these patients, and contribute to our understanding of the biology of normal vision and pineal activity.

Materials and Methods

cDNA Sequencing and RACE

The inventors obtained partial cDNA clones for THC220430 (fetal retina IMAGE 838161, adult retina ATCC 117797, pineal gland IMAGE 232323) and THC90422 (adult retina: ATCC 11795,pineal gland: ATCC 170258, IMAGE 383092) from Research Genetics or ATCC and purified them using the QIAprep spin miniprep kit (QIAGEN). The inventors, then sequenced cDNAs using a primer-walking technique, with the AmpliCycle sequencing kit (Perkin Elmer), and ³²P-labeled primers, beginning with M13 vector primers. Using the human retina Marathon-ready cDNA (Clontech) and the Marathon RACE kit (Clontech), RACE identified the 5′ untranslated region of AIPL1 and obtained the polyadenylation signal of the THC90422 transcripts.

Northern Blot Analysis

The inventors probed a human multiple tissue polyA⁺ RNA Northern blot and a human adult retina total RNA Northern blot at the same time with an amplimer from exon 6 and the 3′ untranslated region of AIPL1 that was ³²P-labeled using the Strip-EZ PCR kit (Ambion). Hybridization was in ULTRA-hyb solution (Ambion) according to the manufacturer's protocols. As a positive control, the inventors incubated both blots with human β-actin using the same reaction conditions.

Retinal/Pineal in situ Hybridization

PCR of a mouse retinal cDNA library using PCR primers designed to the human AIPL1 cDNA (5′-AAGAAAACCATTCTGCACGG-3′ and 5′-TGCAGCTCGTCCAGGTCCT-3′) obtained a 613 bp fragment of mouse AIPL1 cDNA. Sequencing of the resulting fragment using the AmpliCycle Sequencing kit (Perkin Elmer) and ³²p end-labeled primers confirmed that the resulting fragment represented mouse Aipl1 cDNA. The fragment was used as a probe for digoxygenin in situ hybridization using previously described methods (Furukawa, T., Morrow, E. M. & Cepko, C. L. Crx, a novel otx-like homeobox gene, shows photoreceptor-specific expression and regulates photoreceptor differentiation. Cell 91, 531-541 (1997)).

Genomic Sequencing of BAC Clones

The Human BAC I library was screened commercially (Genome Systems) using PCR primer pairs based on the AIPL1 sequence (5′-GACACCTCCCTTTCTCC-3′ and 5′-GCTGGGGCTGCCTGGCTG-3′; 5′-CCGAGTGATTACCAGAGGGA-3′ and 5′-TGAGCTCCAGCACCTCATAG-3′). The inventors purified BAC DNA from the identified clones using the Plasmid Midiprep Kit (QIAGEN) and sequenced it directly using an ABI310 automated sequencer. A primer walking strategy beginning with PCR primers to the cDNA obtained complete intronic sequences. The inventors viewed, edited and aligned sequence data using AutoAssembler (Perkin Elmer) software.

Fluorescence In situ Hybridization

Fluorescence In situ Hybridization (FISH) was performed on normal human chromosome slides prepared by standard cytogenetic procedures. BAC264k12 was labeled with digoxygenin (Boehringer Mannhiem) by nick translation and a probe consisting of 200 ng labeled BAC DNA, 10 μg salmon sperm DNA, 5 μg Human Cot-1 DNA (Gibco BRL) and chromosome 17 alpha satellite DNA labeled with Spectrum Green (Vysis) was denatured and hybridized to denatured slides. Unbound probe was removed by washing in 72° C. 1×SSC buffer and the digoxygenin-labeled DNA was detected with anti-digoxygenin rhodamine (Boehringer Mannheim). Chromosomes were counterstained with 0.2 μg.ml DAPI in an anti-fade solution. Images were captured using the PowerGene probe analysis system (Perceptive Scientific Instruments Inc.).

Radiation Hybrid Panel Mapping

PCR of the STSs originally designed to EST clusters THC220430 and THC90422 in the Stanford G3 radiation hybrid panel confirmed the chromosomal location of AIPL1. The Stanford Human Genome Center RHServer (http://www-shgc.stanford.edu/RH/) interpreted data for chromosomal location.

Patients and Families

All patients gave informed consent prior to their participation in this study. For each case, clinical evaluation was by at least one of the coauthors.

All affected individuals of the original LCA4 family, KC, are affected with Leber congenital amaurosis and bilateral keratoconus. Clinical examination of the affected individuals revealed bilateral ectasia with central thinning of the cornea, before they reached their twenties. The central cornea has a pronounced cone shape with severe corneal clouding. All affected individuals were blind from birth, with absence of rod and cone function as demonstrated by ERG. Patients also show pigmentary deposits in the retina.

All affected individuals of family MD were blind from birth with absence of rod and cone function as demonstrated by ERG, but without keratoconus. Fundus examination indicates pigmentary retinopathy, attenuated blood vessels, and marked macular degeneration.

The two affected individuals of RFS121 had poor central vision from birth, along with severe night blindness and pendular nystagmus. Fundus examination revealed widespread retinal pigment epithelium changes with pigment clumping in the far periphery, severely attenuated retinal vessels, pronounced atrophy within the macula and a pale optic disk. ERG testing in the third decade of life showed non-detectable cone and rod responses.

Affected individuals in family RFS127 also had poor central vision from birth, severe night blindness and pendular nystagmus. Full-field ERGs in the second decade of life revealed non-detectable responses to all stimuli. Fundus examination revealed widespread retinal pigment epithelial changes with pigment clumping, attenuated retinal vessels, macular atrophy and a pale optic disk.

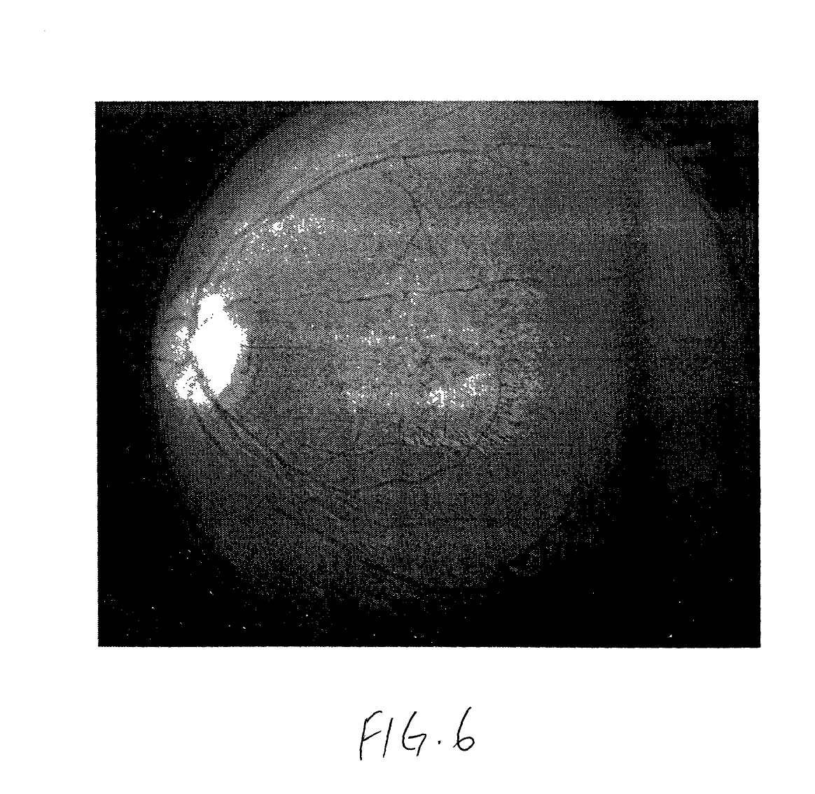

All affected individuals of RFS128 displayed poor central vision from birth, severe night blindness and pendular nystagmus. Cone ERGs to 31 Hz flicker were non-detectable during the first decade of life. A response up to 15 μV to a maximal stimulus flash (presumably rod-mediated) was present during the first decade but borderline detectable by the second decade. Widespread pigment epithelium changes with pigment clumping, attenuated retinal vessels, macular atrophy and pale optic disks were present in affected family members as shown FIG. 6.

Mutation Analysis and Genotyping

The inventors performed direct sequencing for initial mutation analysis, sequencing PCR-amplified AIPL1 exons using a BigDye terminator sequencing kit (Perkin Elmer) on an ABI 310 automated sequencer, according to the manufacturer's protocols.

The inventors performed allele-specific PCR in RFS121 using PCR primers specific to AIPL1 exon 6 sequence, with the forward primer annealing specifically to the wild-type sequence for codon 278 (5′-ACGCAGAGGTGTGGAATG-3′) and the reverse primer in the 3′ untranslated sequence (5′-AAAAAGTGACACCACGATC-3′). The inventors sequenced PCR products as described above.

Primer pairs for microsatellite markers were obtained from Research Genetics. The forward-strand primer was end-labeled with ³²p and polynucleotide kinase (Promega). Amplification, product separation, and visualization were as described previously (Perrault, I. et al. Retinal-specific guanylate cyclase gene mutations in Leber's congenital amaurosis. Nature Genet. 14, 461-464 (1996)). Single stranded conformational analysis was performed at room temperature and 4° C. by previously reported methods (Perrault, I. et al. Retinal-specific guanylate cyclase gene mutations in Leber's congenital amaurosis. Nature Genet. 14, 461-464 (1996)), using directly-sequenced individuals as controls.

Genbank Accession Numbers

Human AIPL1 cDNA and primer sequences, AF148864; mouse Aipl1 partial cDNA sequence AF151392; complete rat Aipl1 cDNA sequence AF180340; partial squirrel monkey Aipl1 genomic sequence AF180341; human genomic AIPL1 sequence AF180472.

Subjects

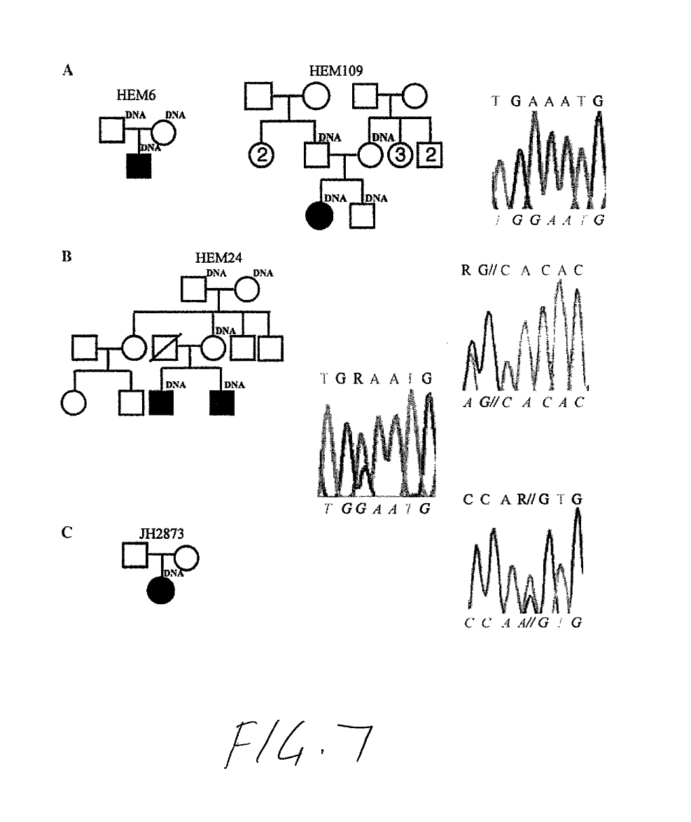

Informed consent was obtained from all subjects. For each case, clinical evaluation was by at least one of the coauthors. Families in which AIPL1 mutations were found are shown in FIGS. 7-9 (old 1-3); individuals from whom DNA samples were obtained and tested are indicated by “DNA” to the upper right of the symbol.

SSCA

Genomic DNA samples from patients were screened by SSCP, by use of six sets of primers as tabulated in Table 5.

TABLE 5
PCR primer sets for amplification
of SSCP fragments of AIPL1

| Amplified Fragment | | Size(s) Analyzed |
|---|---|---|
| (Size [bp]) | Primer Sequences | (bp) |
| Exon 1 (240) | 5′-GGACACCTCCCTTTCTCC-3′ | 240 |
| | 5′-GCTGGGGCTGCCTGGCTG-3′ | |
| Exon 2 (297) | 5′-GGGCCTTGAACAGTGTGTCT-3′ | 151, 146^{a} |
| | 5′-TTTCCCGAAACACAGCAGC-3′ | |
| Exon 3 (364) | 5′-AGTGAGGGAGCAGGATTC-3′ | 210, 154^{a} |
| | 5′-TGCCCATGATGCCCGCTGTC-3′ | |
| Exon 4 (315) | 5′-TTTCGGGTCTCTGATGGG-3′ | 187, 128^{a} |
| | 5′-GCAGGCTCCCCAGAGTC-3′ | |
| Exon 5 (279) | 5′-GCAGCTGCCTCAGGTCATG-3′ | 169, 110^{a} |
| | 5′-GTGGGGTGGAAAGAAAAG-3′ | |
| Exon 6 (497) | 5′-CTGGGAAGGGAGCTGTAG-3′ | 273, 154, 70^{a} |
| | 5′-AAAAGTGACACCACGATCC-3′ | |

^{a}Sizes after restriction digestion.

All primers were synthesized by a commercial source (Genosys Biotechnologies, Woodlands, Tex.). PCR was performed using AmpliTaq Gold Polymerase (Perkin Elmer, Poster City, Calif.). Products were radiolabeled by incorporation of 1 μCi³²P-dCTP, and standard cycling parameters. The annealing step was 62° C. for exon 1, 60° C. for exon 4, 58° C. for exons 2 and 6, 56° C. for exon 5, and 54° C. for exon 3. The amplified fragments of exons 2 and 3 were digested with restriction enzyme NspI prior to electrophoresis; exon 4 was digested with Hsp92II, and exons 5 and 6 were digested with HpaII prior to electrophoresis.

For SSCP, fragments were denatured and separated on a 0.6× MDE gel (Biowhittaker, Rockland, Me.), either at room temperature or at 4° C. The gel was prepared in 0.6× Tris-borate EDTA buffer and was subjected to autoradiography after electrophoresis.

DNA Sequencing

After a novel SSCP variant was identified, a PCR reaction was performed under the same conditions as for SSCP analysis. The fragment was treated with shrimp alkaline phosphatase (Amersham Parmacia, Piscataway, N.J.) and exonuclease I (Amersham Pharmacia, Piscataway, N.J.). Direct sequencing was preformed using a BigDye Terminator Sequencing kit (Perkin Elmer Bioproducts, Foster City, Calif.) on an ABI310 Prism automated sequencer according to the manufacturer's protocols.

For the 12 base-pair deletion in families UTAD231 and UTAD907, a second amplification of the fragment was followed by subcloning with the AdvanTAge PCR Cloning kit (Clontech, Palo Alto). The fragment was then sequenced by use of vector-specific primers. Exact size of deletion was determined by comparison of mutated sequence with the wild-type sequence (GenBank number AF148864).

Results

The six exons of AIPL1 were assayed in 512 unrelated probands with a range of clinical diagnoses (table 2) in order to determine the relative contribution of AIPL1 mutations to LCA and other retinal degenerative diseases. The mutation analysis was performed by single strand conformational polymorphism analysis (SSCP) or by direct sequencing. The inventors identified 12 likely disease-causing mutations in thirteen probands, as is summarized in table 3.

TABLE 3
AIPL1 mutations in this population

| | | | | | Genotype of |
|---|---|---|---|---|---|
| Family | | | | | affected |
| Identifier | Phenotype | Codon^{a} | Mutation | cDNA^{b} | individuals |
| JH3749 | LCA | 79 | M79T | 236T > C (ATG→ACG) | homozygous |
| UCL01 | LCA | 88 | L88X | 264G > A (TGG→TGA) | homozygous |
| HEM115 | LCA | 96 | V96I | 286G > A (GTC→ATC) | heterozygous |
| JH1379 | LCA | 124 | T124I | 341C > T (ACA→ATA) | compound |
| | | | | | heterozygous |
| | | 376 | P376S | 1126C > T (CCG→TCG) | |
| JH3285 | LCA | 163 | Q163X | 487C > T (CAG→TAG) | homozygous |
| HEM26 | LCA | 197 | A197P | 589G > C (GCC→CCC) | homozygous |
| HEM6 | LCA | 278 | W278X | 834G > A (TGG→TGA) | homozygous |
| HEM109 | LCA | 278 | W278X | 834G > A (TGG→TGA) | homozygous |
| HEM24 | LCA | 278 | W278X | 834G > A (TGG→TGA) | compound |
| | | | | | heterozygous |
| | | (93) | IVS2-2 | 277-2 A > G | |
| | | | A > G | | |
| JH2873 | LCA | 278 | W278X | 834G > A (TGG→TGA) | compound |
| | | | | | heterozygous |
| | | 262 | G262S | 784G > A (GGC→AGC) | |
| JH3680 | LCA | 302 | R302L | 905G > T (CGC→CTC) | homozygous |
| UTAD231 | CORD | 351 | P351Δ12 | del1053-1064 | heterozygous |
| UTAD907 | CORD | 351 | P351Δ12 | del1053-1064 | heterozygous |

^{a}Parentheses around a codon indicate the nearest codon to an intronic sequence variant.
^{b}cDNA sequence numbering based on GenBank accession number AF148864, counting the first nucleotide of the first codon as 1.

Leber Congenital Amaurosis (LCA)

Mutation analysis of AIPL1 in 188 probands with LCA identified mutations in 11 families. Due to the proximity of AIPL1 to GUCY2D, it is possible that LCA patients who are identical by descent (IBD) at one locus are also IBD at the other. Therefore, GUCY2D was screened and excluded as a possible cause of the retinal disorder in the 11 families with AIPL1 mutations described below.

Two families (HEM6, Spanish; HEM109, French; as shown in FIG. 7A (old 1A) are homozygous for Trp278X, the mutation identified in the original LCA4 family. If expressed, these alleles are predicted to encode a severely truncated protein. Affected members of the HEM24 family (France) are compound heterozygotes for the Trp278X mutation and a splice-site mutation as shown in FIG. 7B (old 1B). The affected proband of JH2873 is compound heterozygous for Trp278X and for an amino acid substitution, Gly262Ser. None of these mutations were identified in 50 unaffected control individuals or in the other 511 unrelated probands tested.

The remaining mutations were identified in one LCA family each.

Family 3749

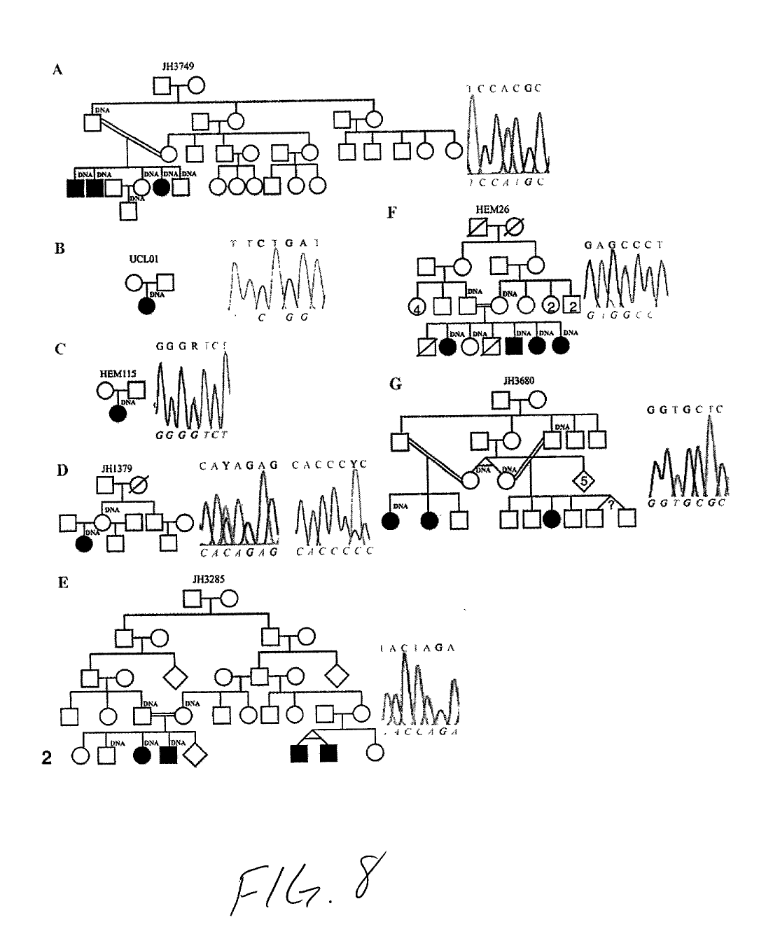

The three affected members of family JH3749 as shown in FIG. 8A(old 2A), from India, were homozygous for an amino acid substitution, Met79Thr. The methionine at this position is conserved in rat and human AIPL1, as well as in AIP. This variant was not identified in the other 511 probands assayed.

Family UCL01

The affected proband of UCL01 as shown in FIG. 8B (old 2B), from Pakistan, is homozygous for a Trp163X mutation. If expressed, these alleles are predicted to encode an AIPL1 protein truncated by more than two-thirds and lacking all three TPR motifs. This mutation was not identified in any of the other 511 unrelated probands tested.

Family HEM115

The affected proband of family HEM115 as shown in FIG. 8C (old 2C), from Portugal, was heterozygous for an amino acid substitution, Val96Ile. The valine at this position is conserved in human and rat aipl1, as well as in human and mouse aip. This variant was not identified in 50 unaffected control individuals or in the other probands tested. Sequencing of the AIPL1 coding sequence failed to identify a second heterozygous mutation in this individual. GUCY2D and CRX, two known causes of LCA were excluded by mutation analysis in this family. It is possible that the second AIPL1 mutation in this individual is within a regulatory region, such as the promoter, or was otherwise not identified by the mutation analysis technique used in this study. The parental DNAs are not available for testing.

Family JH1379

The affected child in the African-American family JH1379 as shown in FIG. 8D (old 2D) is a compound heterozygote for two amino acid substitutions, Thr124Ile and Pro376Ser. The amino acid residue at position 376 is located within the AIPL1 “hinge” region which is only present in primate aipl1; however, the substitution of serine for proline is a nonconservative change. The threonine at position 124 is conserved in human and rat aipl1, and only a conservative change occurs at this position between AIPL1 and AIP.

Family JH3285

The affected individuals of JH3285 as shown in FIG. 8E (old 2E), from Palestine, are homozygous for a Gln163X mutation. If expressed, these alleles are predicted to encode an AIPL1 protein truncated by more than half, with the resulting protein lacking TPR domains II and III. This mutation was not identified in any of the other 511 unrelated probands tested.

Family HEM26

All affected members of family HEM26 from Morocco as shown in FIG. 8F (old 2F) are homozygous for the Ala197Pro mutation. The alanine at this position is conserved in human and rat aipl1, as well as in human and mouse aip. The substitution of a proline for alanine at this position is nonconservative.

Family JH3680

A homozygous Arg302Leu mutation was identified in the affected proband of this family from India as shown in FIG. 8G (old 2G). It is likely that this variant is disease-causing because the arginine at this position is conserved between human, squirrel monkey and rhesus monkey aipl1 (Ma Q, Whitlock J P. A novel cytoplasmic protein that interacts with the Ah receptor, contains tetratricopeptide repeat motifs, and augments the transcriptional response to 2,3,7,8-tetrachlorodibenzo-p-dioxin. J Biol Chem 272:8878-8884, 1997), and the variant was not observed in 50 controls or in the other 511 unrelated probands tested. However, the arginine at this position is not conserved between human and rat aipl1. It is, therefore, possible that this substitution actually represents a rare benign variant. Further studies, such as expression studies, will be necessary to confirm that this mutation significantly alters protein activity and, therefore, is the disease-causing mutation in this family.

Cone-rod Cystrophy, Juvenile RP

Affected probands of two families, UTAD231 and UTAD907, were heterozygous for a 12 bp deletion, Pro351Δ12, within the sequence encoding the “hinge” region of AIPL1. The mutant protein hinge is predicted to lack four amino acids, including two prolines. The amino acids at these positions are identical in the human and rhesus monkey hinge region (unpublished), and this deletion was not observed in 150 control individuals or in the other 510 probands in this study. In addition, DNA samples from two unaffected individuals of family UTAD231 lacked the 12 bp deletion.

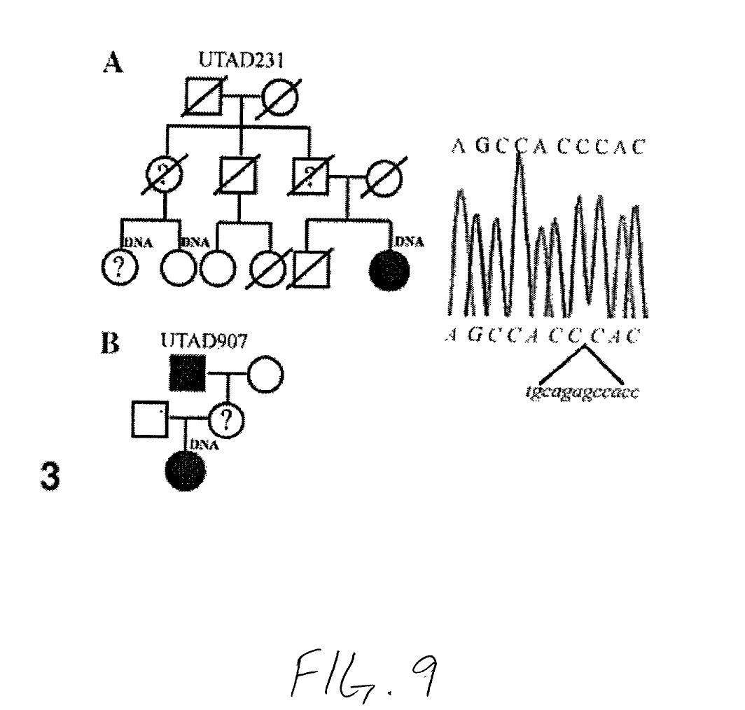

The probands of these families were given the clinical diagnoses of cone-rod dystrophy as shown in FIG. 9A (old 3A) and juvenile RP as shown in FIG. 9B (old 3B), respectively. The pedigrees for these families are small, and DNA samples of additional individuals from these families are unavailable.

Apparently Benign Variants

A number of benign sequence variants or polymorphisms within AIPL1 were identified as shown Table 4.

TABLE 4
Apparently benign sequence variants in this population

| Codon^{a} | Variant | cDNA^{b} | Result | Frequency |
|---|---|---|---|---|
| (33) | IVS1 − 9 G → A | 97 − 9 G > A | Benign, noncoding variant | <0.01 |
| (92) | IVS2 + 66 G → C | 276 + 66 G > C | Benign, noncoding variant | <0.01 |
| (93) | IVS2 − 88 C → T | 277 − 88 C > T | Benign, noncoding variant | <.01 |
| (93) | IVS2 − 14 G → A | 277 − 14 G > A | Benign, noncoding variant | 0.01 |
| (93) | IVS2 − 10 A → C | 277 − 10 A > C | Benign, noncoding variant | 0.55 |
| (156) | IVS3 − 25 T → C | 466 − 25 T > C | Benign, noncoding variant | <0.01 |
| (156) | IVS3 − 21 T → C | 466 − 21 T > C | Benign, noncoding variant | <.01 |
| (262) | IVS5 + 18 G → A | 784 + 18 G > A | Benign, noncoding variant | <.01 |
| 90 | Asp90His (D90H) | 268G > C (GAC → CAC) | Benign coding substitution | 0.16 |
| 37 | Phe37Phe (F37F) | 111T > C (TTT → TTC) | Silent substitution | 0.02 |
| 78 | Ser78Ser (S78S) | 234C > T (TCC → TCT) | Silent substitution | <0.01 |
| 89 | Cys89Cys (C89C) | 287C > T (TGC → TGT) | Silent substitution | 0.01 |
| 100 | Leu100Leu (L100L) | 300G > A (CTG → CTA) | Silent substitution | 0.43 |
| 172 | His172His (H172H) | 516T > C (CAT → CAC) | Silent substitution | <0.01 |
| 217 | Pro217Pro (P217P) | 651G > A (CCG → CCA) | Silent substitution | 0.39 |
| 255 | Asp255Asp (D255D) | 765T > C (GAT → GAC) | Silent substitution | <0.01 |

^{a}Parentheses around a codon indicate the nearest codon to an intronic sequence variant.
^{b}cDNA sequence numbering based on GenBank accession number AF148864 (Sohocki, et al. 2000), counting the first nucleotide of the first codon as 1.

Only one of these variants is predicted to encode an amino acid change, D90H. This variant was identified in several probands, and did not segregate with disease in three families. Several of the variants identified are intronic and located within 30 bp of an intron/exon splice-site, and do not segregate with retinal degeneration in the families. However, these variants must be considered when designing PCR primer sets for mutation screening of AIPL1.

Discussion

The original LCA4 family mutation, Trp278X, is the most common AIPL1 mutation identified in these studies. This mutation is homozygous in affected individuals in three of the thirteen families with an AIPL1 mutation, and is present in affected compound heterozygotes in three additional families. The mutation was identified in affected individuals from multiple populations, including Pakistani, Spanish, French, and American populations. In addition, this study identified ten additional, likely disease-causing mutations in probands affected with LCA.

This study also gives the first evidence that AIPL1 mutations may cause dominant retinal degeneration. The probands of two small families are heterozygous for a 12 bp deletion within the AIPL1 “hinge” region. Interestingly, this deletion occurs adjacent to a predicted casein kinase II (CK2) phosphorylation site, which may be involved in protein complex regulation (as is the CK2 site within the hinge of another FKBP family member, FKBP52; Miyata Y, Chambraud B, Radanyi C, Leclerc J, Lebeau M C, Renoir J M, Shirai R, Catelli M G, Yahara I, Baulieu E E. Phosphorylation of the immunosuppressant FK506-binding protein FKBP52 by casein kinase II: regulation of HSP90-binding activity of FKBP52. Proc Natl Acad Sci 94:14500-5,1997). This mutation was not present in the over 1000 other chromosomes tested for this region and is predicted to significantly alter the protein structure in a region conserved among primates. Sequencing of the entire AIPL1 coding sequence, as well as intron/exon junctions failed to identify a second mutation in these individuals. CRX, a known cause of cone-rod dystrophy (Freund C L, Gregory-Evans C Y, Furukawa T, Papaioannou M, Looser J, Ploder L, Bellingham J, Ng D, Herbrick J A, Duncan A, Scherer S W, Tsui L C, Loutradis-Anagnostou A, Jacobson S G, Cepko C L, Bhattacharya S S, McInnes R R. Cone-rod dystrophy due to mutations in a novel photoreceptor-specific homeobox gene (CRX) essential for maintenance of the photoreceptor. Cell 91:543-53, 1997 and Swain P K, Chen S, Wang Q L, Affatigato L M, Coats C L, Brady K D, Fishman G A, Jacobson S G, Swaroop A, Stone E, Sieving P A, Zack D J. Mutations in the cone-rod homeobox gene are associated with the cone-rod dystrophy photoreceptor degeneration. Neuron 19:1329-36, 1997) was also excluded as the cause of disease in these families. The affected individuals were diagnosed with dominant cone-rod dystrophy or juvenile RP. It will be important to collect DNA samples from additional dominant pedigrees with these diagnoses to confirm the segregation of the mutation with retinal degeneration and to perform expression studies to determine the effect of this mutation on protein structure and activity.

The de1053-1064 (Pro351Δ2) and P376S mutations identified in this study, as well as the Ala336Δ2 mutation identified in the previous study, are all located within the “hinge” region of AIPL1 that is only present in primates. Preliminary data also suggest a high amount of sequence conservation within the hinge region between primates (squirrel monkey, rhesus monkey, and humans). As studies proceed to determine protein function, it is important to determine the role of the hinge in AIPL1 and its significance to primate vision.

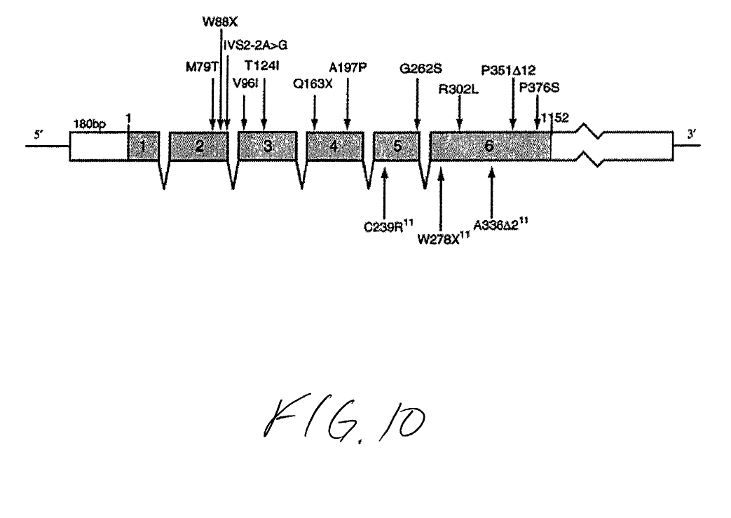

In this study, likely disease-causing AIPL1 mutations were identified in 11 LCA families whose retinal disorder was previously unmapped by linkage. Therefore, combining the data from the previous and current studies, likely disease-causing mutations were identified in 14/202 LCA families. Given these data, the inventors estimate that AIPL1 mutations account for approximately 7% of LCA cases (90% C.L.=0.07±0.02). Further, although there appears to be a “clustering” of retinal disease-causing mutations within one region of some genes, such as RP1 (Bowne S J, Daiger S P, Hims M M, Sohocki M M, Malone K A, McKie A B, Heckenlively J R, Birch D G, Inglehearn C F, Bhattacharya S S, Bird A, Sullivan L S. Mutations in the RP1 gene causing autosomal dominant retinitis pigmentosa. Hum Mol Genet 11:2121-2128, 1999; Guillonneau X, Piriev N I, Danciger M, Kozak C A, Cideciyan A V, Jacobson S G, Farber D B. A nonsense mutation in a novel gene is associated with retinitis pigmentosa in a family linked to the RP1 locus. Hum Mol Genet 8:1541-6, 1999; Pierce E A, Quinn T, Meehan T, McGee T L, Berson E L, Dryja T P. Mutations in a gene encoding a new oxygen-regulated photoreceptor protein cause dominant retinitis pigmentosa. Nat Genet 22:248-254, 1999; and Sullivan L S, Heckenlively J R, Bowne S J, Zuo J, Hide W A, Gal A, Denton M, Inglehearn C F, Blanton S H, Daiger S P. Mutations in a novel retina-specific gene cause autosomal dominant retinitis pigmentosa. Nat Genet22:255-259, 1999), this does not appear to be the case with AIPL1, as the mutations are located throughout the gene as shown in FIG. 10 (old 4).

All references cited herein are incorporated by reference. While this invention has been described fully and completely, it should be understood that, within the scope of the appended claims, the invention may be practiced otherwise than as specifically described. Although the invention has been disclosed with reference to its preferred embodiments, from reading this description those of skill in the art may appreciate changes and modification that may be made which do not depart from the scope and spirit of the invention as described above and claimed hereafter.

We claim:

1. A composition comprising a polynucleotide sequence, wherein the polynucleotide sequence comprises an AIPL1 sequence within the LCA4 region of chromosome 17p13 and is selected from the group consisting of a wild-type AIPL1 sequence and a mutant AIPL1 sequence.

2. The composition of claim 1, wherein the mutants are selected from the group consisting of Ala336Δ2, Trp278X, Cys239Arg, M79T, L88X, V96I, T124I, P376S, Q163X, A197P, IVS2-2, G262S, R302L, P351D12, Cys42X (TGT→TGA), Val33ins 8 bp (GTGATCTT), Leu257del 9 bp (CTCCGGCAC) and mixtures and combinations thereof.

3. A protein comprising SEQ. ID. NOs. 72-78 and variants of the protein of SEQ. ID. NO. 72, or a polypeptide expressed by a polynucleotide comprising a nucleotide sequence selected from the group consisting of SEQ. ID NOs. 1-8 or mutants of SEQ. ID. NO. 1 selected from the group consisting of SEQ. ID No. 9-41.

4. A purified polynucleotide sequence comprising a sequence selected from the group consisting of SEQ ID NOs. 1-71.

5. A retinal disease diagnostic library comprising anti-sense DNA sequences, each sequence corresponding to a DNA sequence including a mutation of the AIPL1 gene selected from the group consisting of SEQ. ID Nos. 9-41 and mixtures and combinations thereof.

6. A primer comprising an AIPL1 sequence, wherein the AIPL1 sequence is selected from the group consisting of a wild-type AIPL1 sequence and a mutant AIPL1 sequence, wherein the mutant-AIPL1 contributes to a retinal disease.

7. The primer of claim 6, further comprising a polynucleotide sequence selected from the group consisting of SEQ ID NOs. 42-47 and 60-71.

8. A probe comprising an AIPL1 sequence, wherein the AIPL1 sequence is selected from the group consisting of a wild-type AIPL1 sequence and a mutant AIPL1 sequence, wherein the mutant-AIPL1 contributes to a retinal disease.

9. A method to determine if an animal has a retinal disease or has a propensity to pass a retinal disease to offspring, comprising the steps of:
(a) extracting polynucleotide from a cell or sample;
(b) determining if the polynucleotide contains a mutation in an AIPL1 encoding or regulating region; and
(c) correlating the presence of the mutation as an indication of a retinal disease or a propensity to pass a retinal disease to offspring.

10. The method of claim 9, further comprising the steps of:
obtaining a patient sample; and
amplifying the polynucleotide.

11. The method of claim 10, wherein the amplifying is done via polymerase chain reaction.

12. The method of claim 9, wherein the determining is done via polynucleotide sequence.

13. The method of claim 9, wherein the mutations are selected from the group consisting of Ala336Δ2, Trp278X, Cys239Arg, M79T, L88X, V96I, T124I, P376S, Q163X, A197P, IVS2-2, G262S, R302L, P351D12, Cys42X (TGT→TGA), Val33ins 8 bp (GTGATCTT), Leu257del 9 bp (CTCCGGCAC) and mixtures and combinations thereof.

14. A therapeutic method to treat retinal disease comprising the step of administering to an animal an effective amount of a protein encoded by a wild-type AIPL1 gene or a polynucleotide sequence a wild-type AIPL1 gene or a retinal medication designed to ameliorate disease symptoms to the patient if the mutation is detected or mixtures or combinations thereof.

15. The method of claim 14, wherein the medication is an drug that inhibits retinal cell death.

16. The method of claim 14, wherein the mutations are selected from the group consisting of Ala336Δ2, Trp278X, Cys239Arg, M79T, L88X, V96I, T124I, P376S, Q163X, A197P IVS2-2, G262S, R302L, P351D12, Cys42X (TGT→TGA), Val33ins 8 bp (GTGATCTT), Leu257del 9 bp (CTCCGGCAC) and mixtures and combinations thereof.

17. A method to determine if a patient has a mutant AIPL1 gene comprising:
(a) extracting AIPL1 polypeptide from a cell or sample from the patient;
(b) determining if the polypeptide contains an AIPL1 mutation; and
(c) correlating the mutation as an indication of a retinal disease.

18. The method of claim 17, wherein the mutations are selected from the group consisting of Ala336Δ2, Trp278X, Cys239Arg, M79T, L88X, V96I, T124I, P376S, Q163X, A197P IVS2-2, G262S, R302L, P351D12, Cys42X (TGT ->TGA), Val33ins 8 bp (GTGATCTT), Leu257del 9 bp (CTCCGGCAC) and mixtures and combinations thereof.

19. A method of producing a cell expressing an AIPL1 mutation comprising transfecting a cell with a polynucleotide sequence having at least one AIPL1 mutation in the sequence.

20. The method of claim 19, wherein the encoded mutation is selected from the group consisting of are selected from the group consisting of Ala336Δ2, Trp278X, Cys239Arg, M79T, L88X, V96I, T124I, P376S, Q163X, A197P, IVS2-2, G262S, R302L, P351D12, Cys42X (TGT→TGA), Val33ins 8 bp (GTGATCTT), Leu257del 9 bp (CTCCGGCAC) and mixtures and combinations thereof.

21. A method for determining the presence of an AIPL1 mutant in a patient sample, which comprises:
(a) isolating polynucleotide extracted from the patient sample;
(b) hybridizing a detectably labeled oligonucleotide to the polynucleotide isolated in step (b), the oligonucleotide having at its 3′ end at least 15 nucleotides complementary to a wild type polynucleotide sequence having at least one mutation;
(c) attempting to extend the oligonucleotide at its 3′-end;
(d) ascertaining the presence or absence of a detectably labeled extended oligonucleotide; and
(e) correlating the presence or absence of a detectably labeled extended oligonucleotide in step (e) with the presence or absence of a AIPL1 mutation.

22. The method of claim 21, further comprising taking a patient sample prior to the isolating step.

23. The method of claim 21, wherein the isolated nucleic acid is amplified prior to hybridization.

24. The method of claim 21, wherein the detectable label on the oligonucleotide is an enzyme, radioisotope or fluorochrome.

25. A test kit useful for the detection of AIPL1 mutations comprising a container containing at least one polynucleotide capable of hybridizing with a polynucleotide encoding at least one mutation selected from the group consisting of Ala336Δ2, Trp278X, Cys239Arg, M79T, L88X, V96I, T124I, P376S, Q163X, A197P, IVS2-2, G262S, R302L, P351D12, Cys42X (TGT→TGA), Val33ins 8 bp (GTGATCTT), Leu257del 9 bp (CTCCGGCAC) and mixtures and combinations thereof.

26. A method of screening compounds to determine their effectiveness in counteracting a cell's retinal behavior due to a mutation in its AIPL1 gene comprising:
(a) contacting the compound with a cell including a mutation is its AIPL1 gene where the mutation is selected from the group consisting of Ala336Δ2, Trp278X, Cys239Arg, M79T, L88X, V96I, T124I, P376S, Q163X, A197P, IVS2-2, G262S, R302L, P351D12, Cys42X (TGT→TGA), Val33ins 8 bp (GTGATCTT), Leu257del 9 bp (CTCCGGCAC) and mixtures and combinations thereof; and
(b) determining if the cell is affected by the compound.

27. A method to determine if a cell or sample has an AIPL1 mutation comprising:
(a) extracting polynucleotide from a cell;
(b) amplifying polynucleotides which encode AIPL1; and
(c) determining if the polynucleotide contains a mutation;
(d) correlating the presence of the mutation as an indication of a retinal disease or a propensity to pass a retinal disease to offspring.
